(19) United States
(12) Patent Application Publication
(10) Pub. No.: US 2025/0188084 A1
(43) Pub. Date: Jun. 12, 2025

(54) COMPOUNDS AND METHODS FOR MODULATING CDK9 ACTIVITY

(71) Applicant: KRONOS BIO, INC., San Mateo, CA (US)

(72) Inventors: WES TROTTER, San Mateo, CA (US); MARIUS POP, San Mateo, CA (US); ZHIHUA MA, San Mateo, CA (US); DAVID FREEMAN, San Mateo, CA (US)

(73) Assignee: KRONOS BIO, INC., San Mateo, CA (US)

(21) Appl. No.: 18/034,634

(22) PCT Filed: Nov. 4, 2021
(86) PCT No.: PCT/US2021/058014

§ 371 Date: Apr. 28, 2023

Related U.S. Application Data
(60) Provisional application No. 63/110,303, filed on Nov. 5, 2020.

(51) Int. Cl. C07D 487/04

(52) U.S. Cl. CPC C07D 487/04

(57) ABSTRACT

Provided are heterobifunctional degrader compounds that bind with cyclin dependent kinase 9 (CDK9) and E3 ubiquitin ligase. Also provided are methods of treating a subject for a CDK9-mediated disease by administering a therapeutically effective dose of a pharmaceutical composition including the heterobifunctional degrader.

### INTRODUCTION

The cyclin-dependent kinase (CDK) family of proteins are key regulators of the cell cycle and gene transcription. The cell cycle is a regulatory cellular mechanism for the timing of cell growth and division. The cell cycle is a multipronged process that directs cellular proliferation through a series of checkpoints that correct for DNA damage, genetic derangements, and other errors. Nonhuman Primates in Biomedical Research (Second Edition, 2012). Each stage is controlled by a combination of cyclins and CDKs, where the CDKs phosphorylate a specific set of cyclins to trigger entry into the next stage of the cell cycle. Casem, M. L., Case Studies in Cell Biology, ISBN-13: 978-0128013946, 2016, Chapter: Cell Cycle (p. 299-326). Accumulation of cyclin proteins through regulation of cyclin mRNA transcription function as “biological switches” to turn CDKs on and off and move the cell from one stage to the next.

CDKs 1, 2, 3, 4 and 6 regulate time of the cell division cycle while CDK 7 and CDK 9 regulate the activity of transcription through regulation of RNA polymerase II via phosphorylation of its carboxy terminal domain. Lucking, et al., ChemMedChem, 2017, 12, 1776-1793, doi:10.1002/cmdc.201700447.

CDK9 controls the transcriptional activity of key oncogenic proteins such as AR, MYC, MCL-1, and BCL-2 and stimulates pro-inflammatory transcription factors such as NFkB and STAT3. Gregory et al., Leukemia. 2015 June; 29(6): 1437-1441; Krystof, et al., Curr Pharm Des. 2012 July; 18(20): 2883-2890. CDK9 forms a heterodimer with one of four cyclin partners (cyclin T1, cyclin K, cyclin T2a, or cyclin T2b) called positive transcription elongation factor (P-TEFb). RNA polymerase II pauses mRNA transcription after 20-40 nucleotides along the DNA template due to interaction of negative elongation factors which serve as a major regulatory control mechanism for transcription of rapidly induced genes. P-TEFb overcomes pausing of RNA polymerase II by phosphorylation of the carboxy terminal domain of RNA polymerase II, and inactivation of negative elongation factors. Compounds targeting CDK9 and P-TEFb are currently undergoing clinical study. The enzymatic activity of CDK9 is important for stimulating transcription elongation of most protein coding genes. Krystof, et al., Curr Pharm Des. 2012 July; 18(20): 2883-2890. A number of CDK inhibitors with heterocyclic core structures have been developed. For example, purine scaffolds have been the source of CDK inhibitors developed for treating cancer, including seliciclib (Cyclacel Pharmaceuticals, Inc) and other purine derivatives. S. C. Wilson et al., Bioorg & Med Chem 2011 November; 19(22): 6949-6965. Besides CDK9 these purine derivatives also target CDK7 and CDK2, whereas CDK2 inhibition causes safety and toxicity concerns. CDK9 inhibitors based on a triazine core have also been developed, e.g., Atuveciclib. Lucking et al., ChemMedChem 2017, 12, 1776-1793. Unfortunately, treatment with CDK9 inhibitors remains relatively unsuccessful and involves many adverse effects. Morales et al., Cell Cycle 2016, vol. 15, no. 4, 519-527. Therefore, a need exists for new CDK9 inhibitors for treating diseases mediated by CDK9.

### SUMMARY

Provided are heterobifunctional degrader compounds that bind with cyclin dependent kinase 9 (CDK9) and E3 ubiquitin ligase. Also provided are methods of treating a subject for a CDK9-mediated disease by administering a therapeutically effective dose of a pharmaceutical composition including the heterobifunctional degrader.

### DETAILED DESCRIPTION

Provided are heterobifunctional degrader compounds that bind with cyclin dependent kinase 9 (CDK9) and E3 ubiquitin ligase. Also provided are methods of treating a subject for a CDK9-mediated disease by administering a therapeutically effective dose of a pharmaceutical composition including the heterobifunctional degrader.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a droplet” includes a plurality of such droplets and reference to “the discrete entity” includes reference to one or more discrete entities, and so forth.

It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

“Linker” and “linking group” are used interchangeably to refer to a group that connects to two groups. Exemplary types of connections include covalent bonds. As used herein, the term “radical”, such as in “monoradical” or “diradical”, refers to the number groups that a group can connect with. For instance, a monoradical group can connect to only a single other group, e.g., the methyl (—CH₃) and ethyl (—CH₂CH₃) groups are monoradical groups. In contrast, the —CH₂— and —CH₂CH₂— groups are diradical groups since they can each connect to two different groups. Since a linker connects two groups, a linker is a diradical group. Connections between groups can also be described by the term “valent”, such as in “monovalent” or “divalent”, which refers to the bond order of the connection. For instance, the group —CH₃ is a monovalent group since it can form a single covalent bond with another group, e.g., with —OH to form H₃COH. The group ═CH₂ is a divalent group since it can form a double bond with another group, e.g., with an oxygen atom to form formaldehyde (CH₂O).

“Alkyl” refers to monovalent saturated aliphatic hydrocarbyl groups having from 1 to 10 carbon atoms and such as 1 to 6 carbon atoms, or 1 to 5, or 1 to 4, or 1 to 3 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain (except the C₁ carbon atom) have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic. For instance, a —CH₂CH₂CH₃ (propyl) group can be considered an alkyl group. If the second carbon atom is replaced with an oxygen atom, the resulting group would be —CH₂OCH₃. This group can be considered as a substituted alkyl because it can be thought of as a —CH₃ (methyl) group where one of the hydrogen atoms is substituted with a —OCH₃ (methoxy) group.

“Alkylene” refers to divalent aliphatic hydrocarbyl groups preferably having from 1 to 6 and more preferably 1 to 3 carbon atoms that are either straight-chained or branched, and which are optionally interrupted with one or more groups selected from —O—, —NR¹⁰—, —NR¹⁰C(O)—, —C(O)NR¹⁰— and the like. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Substituted alkylene” refers to an alkylene group having from 1 to 3 hydrogens replaced with substituents as described for carbons in the definition of “substituted” below.

The term “alkane” refers to alkyl group and alkylene group, as defined herein.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH).

The term “substituted alkynyl” refers to an alkynyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, and —SO₂-heteroaryl.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Cyano” or “nitrile” refers to the group —CN.

Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkenyl” refers to non-aromatic cyclic alkyl groups of from 3 to 10 carbon atoms having single or multiple rings and having at least one double bond and preferably from 1 to 2 double bonds.

The term “substituted cycloalkenyl” refers to cycloalkenyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO— alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkynyl” refers to non-aromatic cycloalkyl groups having single or multiple rings and having at least one triple bond.

“Halo” or “halogen” refers to fluoro, chloro, bromo, and iodo.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic. To satisfy valence requirements, any heteroatoms in such heteroaryl rings may or may not be bonded to H or a substituent group, e.g., an alkyl group or other substituent as described herein. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from nitrogen, sulfur, or oxygen, where, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties. To satisfy valence requirements, any heteroatoms in such heterocyclic rings may or may not be bonded to one or more H or one or more substituent group(s), e.g., an alkyl group or other substituent as described herein.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO— heteroaryl, —SO₂-alkyl, —SO₂-substituted alkyl, —SO₂-aryl, —SO₂-heteroaryl, and fused heterocycle.

“Oxo” refers to the atom (═O).

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O—M⁺, —SO₂OR⁷⁰, —OSO₂R⁷⁰, —OSO₂O—M⁺, —OSO₂OR⁷⁰, —P(O)(O⁻)₂(M⁺)₂, —P(O)(OR⁷⁰)O-M⁺, —P(O)(OR⁷⁰) 2, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O) O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸OR⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S³¹ M⁺, —NR⁸⁰R⁸⁰, trihalomethyl, —CF₃, —CN, —NO, —NO₂, —S(O)₂R⁷⁰, —S(O)₂O⁻M*, —S(O)₂OR⁷⁰, —OS(O)₂R⁷⁰, —OS(O)₂ O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S) R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)—O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

It will be appreciated that the term “or a salt or solvate or stereoisomer thereof” is intended to include all permutations of salts, solvates and stereoisomers, such as a solvate of a pharmaceutically acceptable salt of a stereoisomer of subject compound.

### Compounds

Provided are heterobifunctional degrader compounds that bind with cyclin dependent kinase 9 (CDK9) and E3 ubiquitin ligases. In some cases, the compound is of formula (I):

X-L-Y (I)
wherein:
X is an E3 ubiquitin ligase binding ligand; and
L is a linking group covalently bonded to X and Y;
Y is a cyclin dependent kinase 9 (CDK9) binding ligand of formula (II):

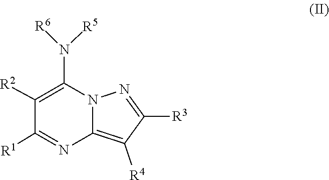

or a pharmaceutically acceptable salt thereof;
   R¹ is C1-C6 alkyl, C3-C6 cycloalkyl, C3-C6 cycloalkenyl, alkenyl, alkynyl, aryl, heteroaryl, or heterocyclyl, optionally substituted at any position with one or more of D, halo, R⁷CO₂R⁸, CO₂R⁸, CO₂H, R⁷CO₂H, NH₂, NHR⁸, OH, OR⁸, SH, SR⁸, NHCOR⁸, NHSO₂R⁸, SO₂NH₂, SO₂NHR⁸,
   or R¹ and R² together form a fused C5-C6 cycloaryl, optionally substituted at any position with one or more of D, halo, NH₂, NHR⁸, NR⁷R⁸, OH, OR⁸, SH, SR⁸, NHCOR⁸, NHSO₂R⁸, SO₂NH₂, or SO₂NHR⁸;
   R², R³, and R⁵ are independently H, D, halo, or C1-C5 alkyl or C3-C6 cycloalkyl optionally substituted at any position with one more of D, halo, R⁷CO₂R⁸, CO₂R⁸, CO₂H, R⁷CO₂H, NH₂, NHR⁸, OH, OR⁸, SH, SR⁸, NHCOR⁸, NHSO₂R⁸, SO₂NH₂, SO₂NHR⁸,
   or R² and R⁵ together form a fused C5-C6 cycloaryl, optionally substituted at any position with one or more of D, halo, R⁷CO₂R⁸, CO₂R⁸, CO₂H, R⁷CO₂H, NH₂, NHR⁸, OH, OR⁸, SH, SR⁸, NHCOR⁸, NHSO₂R⁸, SO₂NH₂, SO₂NHR⁸;
   R⁴ is H, D, halo, C1-C5 alkyl, C3-C6 cycloalkyl, cyano, hydroxyl, or —O—(C1-C5 alkyl), optionally substituted at any position with one more of D, halo, R⁷CO₂R⁸, CO₂R⁸, CO₂H, R⁷CO₂H, NH₂, NHR⁸, OH, OR⁸, SH, SR⁸, NHCOR⁸, NHSO₂R⁸, SO₂NH₂, SO₂NHR⁸;
   R⁶ is H or D;
   R⁷ is (CH₂)ₙ and n is an integer from 1 to 6; and
   R⁸ is C1-C6 alkyl or C3-C6 cycloalkyl, optionally substituted at any position with one or more of D, halo, OH, SH, or NH₂.

### Group Y

Y is a cyclin dependent kinase 9 (CDK9) binding ligand of formula (II). “CDK9” as used herein refers to a protein encoded by CDK9 gene. CDK refers to cyclin-dependent kinase, wherein “cyclin” refers to regulation of the cell cycle and “kinase” indicates a protein that functions as an enzyme by phosphorylating another protein. Several CDK genes and proteins are known, wherein the particular CDK being discussed herein has been named CDK9. Additional names for CDK9 include CDC2L4, PITALRE, C-2k, and TAK. In some cases the CDK9 protein is encoded by a human CDK9 gene located at band 9q34.11 of chromosome 9. One function of CDK9 is to control the transcriptional activity of key oncogenic proteins such as AR, MYC, MCL-1, and BCL-2 and stimulates pro-inflammatory transcription factors such as NFkB and STAT3 (Gregory et al., Leukemia. 2015 June; 29(6): 1437-1441; Krystof, et al., Curr Pharm Des. 2012 July; 18(20): 2883-2890). CDK9 can form a heterodimer with one of four cyclin partners (cyclin T1, cyclin K, cyclin T2a, or cyclin T2b) called positive transcription elongation factor (P-TEFb).

In some cases, Y specifically binds to a CDK9. As used herein, “specific binding” refers to the ability of a first group (or a first member of a specific binding pair) to preferentially bind to a particular analyte (or a second member of a specific binding pair) in a mixture of different analytes. For example, Y might preferentially bind to CDK9 over CDK4, i.e. Y selectively binds to CDK9 over CDK4. In some cases, the binding preference is 5-fold or more, such as 10-fold or more or 100-fold or more. The term “binding ligand” means that a group is capable of binding to another group, e.g. group Y is capable of binding to a CDK9.

In some cases, Y is covalently attached to L through R⁵. For example, R⁵ can be an optionally substituted C3-C6 cycloalkyl group. For instance, R⁵ can be a substituted C3-C6 cycloalkyl group. In some cases, R⁵ is an optionally substituted C5 cycloalkyl group, e.g. a substituted C5 cycloalkyl group. In some cases, R⁵ has a structure selected from the group consisting of:

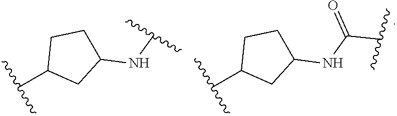 [SMILES: *NC1CCC(*)C1 ; *C(=O)NC1CCC(*)C1]

In some cases, Y is covalently attached to L through R¹, R², R³, R⁴, or R⁶. In some cases Y is covalently attached to L through the 5-membered ring of formula (II), i.e., R³ or R⁴ is absent, and there is a covalent bond at the R³ or R⁴ location to L. In other words, R³ or R⁴ can be considered a hydrogen atom, but that hydrogen atom can be removed, thereby providing a location for covalent bonding to L. In some cases Y is covalently attached to L through the 6-membered ring of formula (II), i.e., R¹ or R² is absent, and there is a covalent bond at the R¹ or R² location to L.

As discussed above, R¹ is a C1-C6 alkyl, C3-C6 cycloalkyl, alkenyl, alkynyl, aryl, heteroaryl, or heterocyclyl, and optionally substituted. In some embodiments, R¹ is heterocyclyl, e.g., heterocycloalkyl or heterocycloalkenyl. Exemplary heterocycloalkenyl groups include structures derived from dihydrofuran, dihydropyran, or dihydropyrrole by removal of a hydrogen atom. Exemplary heterocyloalkyl groups include structures derived from tetrahydrofuran, tetrahydropyran, and tetrahydropyrrole by removal of a hydrogen atom. In some cases, R¹ is a C1-C6 alkyl group. In some cases, R¹ has a structure selected from the group consisting of:

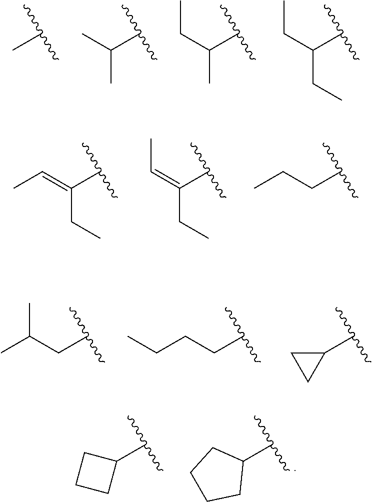

In some cases, R², R³, and R⁶ are each independently H or D. For example, in some cases R², R³, and R⁶ are each H. In other cases, one or more of R², R³, and R⁶ are D, e.g. R² is H, R³ is D, and R⁶ is D. In some cases, one or both of R² and R³ are independently C1-C5 alkyl or C3-C6 cycloalkyl optionally substituted at any position with one more of D, halo, R⁷CO₂R⁸, CO₂R⁸, CO₂H, R⁷CO₂H, NH₂, NHR⁸, OH, OR⁸, SH, SR⁸, NHCOR⁸, NHSO₂R⁸, SO₂NH₂, SO₂NHR⁸.

In some embodiments, R⁴ is selected from the group consisting of halo, cyano, C1-C5 alkyl, hydroxyl, —O—(C1-C5 alkyl), and fluorinated C1-C5 alkyl. C1-C5 alkyl refers to alkyl groups that have 1, 2, 3, 4, or 5 carbon atoms. Fluorinated C1-C5 alkyl refers to a C1-C5 alkyl group wherein one or more hydrogen atom is replaced with a fluorine atom, e.g., —CH₂F, CHF₂, and —CF₃. Exemplary —O—(C1-C6 alkyl) groups include methoxy and ethoxy. In some cases, R⁴ is halo, cyano, or methyl. In some cases, R⁴ is halo, e.g. chloro. In other cases, R⁴ is H or D.

In some cases, Y has a structure selected from the group consisting of:

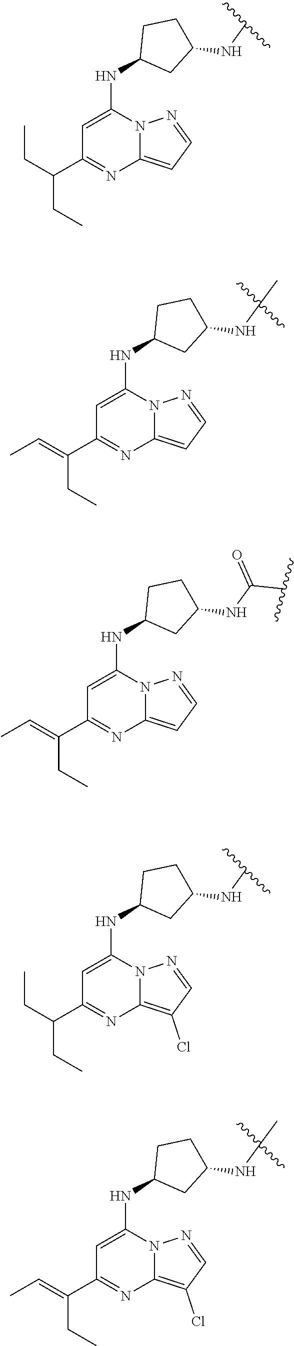 [SMILES: *N[C@H]1CC[C@H](Nc2cc(C(CC)CC)nc3ccnn23)C1 ; *N[C@H]1CC[C@H](Nc2cc(/C(=C/C)CC)nc3ccnn23)C1 ; *C(=O)N[C@H]1CC[C@H](Nc2cc(/C(=C/C)CC)nc3ccnn23)C1 ; *N[C@H]1CC[C@H](Nc2cc(C(CC)CC)nc3c(Cl)cnn23)C1 ; *N[C@H]1CC[C@H](Nc2cc(/C(=C/C)CC)nc3c(Cl)cnn23)C1]

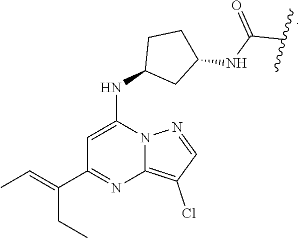 [SMILES: *C(=O)N[C@H]1CC[C@H](Nc2cc(/C(=C/C)CC)nc3c(Cl)cnn23)C1]

Additional exemplary Y groups are described in U.S. Patent Application Publication 2020/0131189 (hereinafter the '189 publication), which is incorporated herein by reference. Formula (I) of the '189 publication describes compounds of a formula that has the same generic structure and numbering recited in Formula (II) of the present application. For instance, the Y group can include a structure of any one of compounds 1 through 208 of the '189 publication. In other words, a hydrogen atom from any one of compounds 1 through 208 of the '189 publication can be removed, and the resulting monovalent group can be bonded to the L group at the location of the removed hydrogen atom. For example, compound 1 of the '189 publication is shown below, and a hydrogen atom (which is not shown) can be removed, providing for a location to connect the Y group to the L group. For instance, in the first and second options shown below, a hydrogen atom that was located on the cyclopentane ring was removed, and the resulting monovalent group can be a Y group that connects to the L group at the location of the removed hydrogen atom. In the third option, a hydrogen atom on the pyrazole ring was removed, providing for a connection to the L group. In some cases, the Y group is a derivative of any one of compounds 1 through 208 of the '189 publication, e.g., wherein the connection to the L group is through a cycloalkyl group bonded to the amino group, e.g. as shown in options 1 and 2 below.

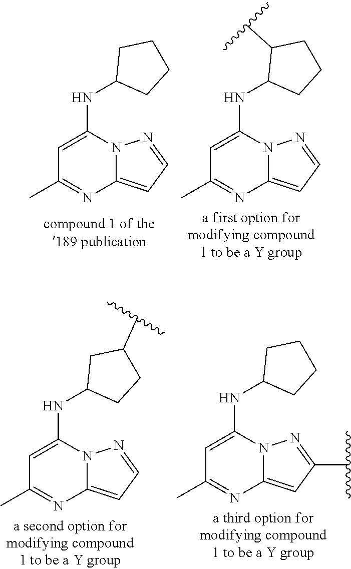

As described above, X is an E3 ubiquitin ligase binding ligand. In other words, it has the ability to bind to an E3 ubiquitin ligase. This binding, and its potential use in treating a patient for a medical condition, can be understood by considering the role of the E3 ubiquitin ligase in the biochemical process known as the Ubiquitin Proteasome System (UPS).

The Ubiquitin Proteasome System (UPS) helps to regulate protein homoeostasis by degrading proteins, e.g. proteins that are damaged or misfolded. The UPS system involves a sequence of enzymatic events that are referred to as E1, E2, and E3. During the E1 step, the protein ubiquitin is activated such that it forms a covalent intermediate with the E1 enzyme. Next, the ubiquitin protein is transferred from the E1 enzyme to the E2 enzyme (i.e., the E2 ubiquitin-conjugating enzyme). In the final step, the E3 enzyme (i.e., the E3 ubiquitin ligase) catalyzes the transfer of the ubiquitin from E2 to the protein that is destined for degradation. Afterwards, the presence of the ubiquitin, or a polyubiquitin chain, on the target protein is recognized by a separate element called proteosomes, which actually degrade the target protein. As such, the term “E3 ubiquitin ligase” refers to an enzyme that catalyze the transfer of a ubiquitin group from another enzyme, e.g. the E2 ubiquitin-conjugating enzyme, to a target protein, e.g. wherein the target protein is degraded by a proteasome upon detection of one or more ubiquitin groups.

As such, since the X group of the present compounds is capable of binding to a part of the E3 ubiquitin ligase, this binding allows the compounds to influence the action Ubiquitin Ligase System in some cases, such as using the UPS to selectively degrade certain proteins, which can be used in medical treatment of a patient.

In some cases, X is a cereblon (CRBN) binding ligand, wherein CRBN is a protein encoded by the CRBN gene. Additional names for CRBN include MRT2 and MRT2A (Mental Retardation, Non-Syndromic, Autosomal Recessive, A2). The role of CRBN in the UPS degradation of proteins occurs when CRBN joins other proteins to form the E3 enzyme complex, i.e. the “E3 ubiquitin ligase complex”. In some cases CRBN joins with Cullins-4A (CUL4A), regulator of cullins 1 (ROC1), and damaged DNA binding protein 1 (DDB1) to form the E3 ubiquitin ligase complex. As such, since the X group of the present compounds has the ability to bind to cereblon (CRBN), this interaction can in some cases bring the compounds into proximity with the E3 ubiquitin ligase complex as a whole. In some cases, X binds to a CRBN protein that is encoded by a human CRBN gene located at band 3p26.2 of chromosome 3.

In some cases, X is a CRBN binding ligand that is a small molecule group, e.g. it has 200 atoms or less. For example, X can have a structure derived by removal of a hydrogen atom from a structure selected from the group consisting of:

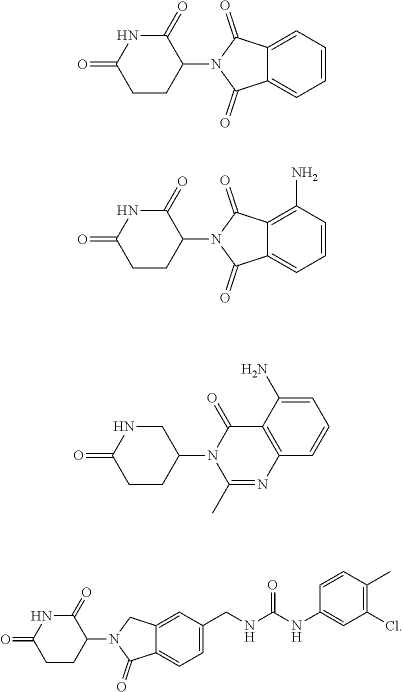 [SMILES: O=C1CCC(N2C(=O)c3ccccc3C2=O)C(=O)N1 ; Nc1cccc2c1C(=O)N(C1CCC(=O)NC1=O)C2=O ; Cc1nc2cccc(N)c2c(=O)n1C1CCC(=O)NC1 ; Cc1ccc(NC(=O)NCc2ccc3c(c2)CN(C2CCC(=O)NC2=O)C3=O)cc1Cl]

The X group can connect L at any suitable point. For example, a hydrogen atom at any one of the locations marked with the asterisk (*) in the structure below can be removed, thereby providing a location for a covalent bond to L. As such, in some cases the X group connects to the L group through an aryl carbon, through an alkyl carbon, or through a nitrogen atom.

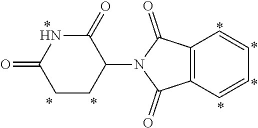 [SMILES: O=C1CCC(N2C(=O)c3ccccc3C2=O)C(=O)N1]

As another example, the X group can be derived from the molecule shown below, e.g., wherein the connection to L can be through the group shown as —NH₂. In other cases, the X group has the structure shown below with the exception that a hydrogen atom corresponding to one of those marked with an asterisk (*) in the above example is removed, providing a location for the bond to L.

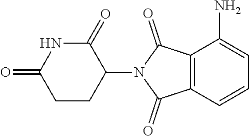 [SMILES: Nc1cccc2c1C(=O)N(C1CCC(=O)NC1=O)C2=O]

As such, the X group can have a structure derived from the four CRBN binding ligands shown above, wherein one hydrogen atom is removed, providing a location for covalent binding to L. In some cases, the X group has the formula

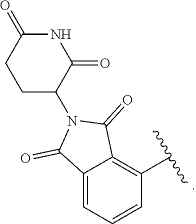 [SMILES: *c1cccc2c1C(=O)N(C1CCC(=O)NC1=O)C2=O]

In addition to cereblon (CRBN), Von Hippel-Lindau (VHL) is another protein involved with E3 ubiquitin ligase. The VHL protein is encoded by the VHL gene, and is sometimes also referred to as the “Von Hippel-Lindau tumor suppressor” since mutation in the VHL gene can cause cancerous tumors. In some cases, X binds to a VHL protein encoded by a human VHL gene located at band 3p25.3 of chromosome 3. In relation to the Ubiquitin Proteasome System (UPS), VHL can join with other proteins to form an E3 ubiquitin ligase, e.g. by joining with elongin B, elongin C, and cullin-2. Additional names for the VHL protein include HRCA1, RCA1, and VHL1. Thus, since the VHL protein is part of an E3 ubiquitin ligase, if X is a VHL binding ligand, this binding can bring the compounds described in the present application into proximity with the E3 ubiquitin ligase, potentially allowing for alteration or use of that ligase, such as for medical treatment.

In some cases, X is a Von Hippel-Lindau (VHL) binding ligand. In some cases, X is a VHL binding ligand that is a small molecule group, e.g. it has 200 atoms or less. For instance, X can have the structure derived by removal of a hydrogen atom from a structure shown below.

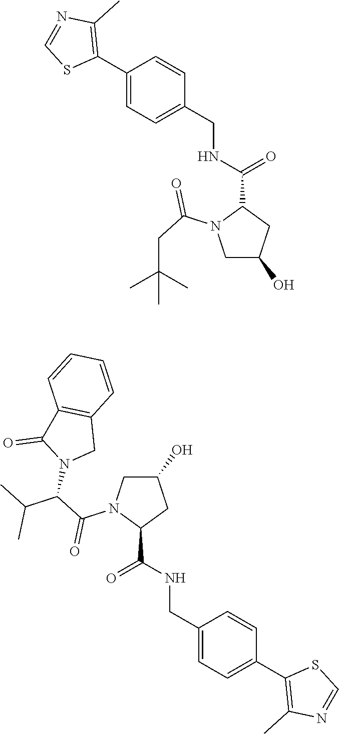

X can be covalently bonded to L at any suitable location. For instance, a hydrogen atom at any of the locations marked with an asterisk (*) can be removed, providing a location for binding to L. In some cases, X connects to L through an aryl carbon, an alkyl carbon, a nitrogen atom, or an oxygen atom.

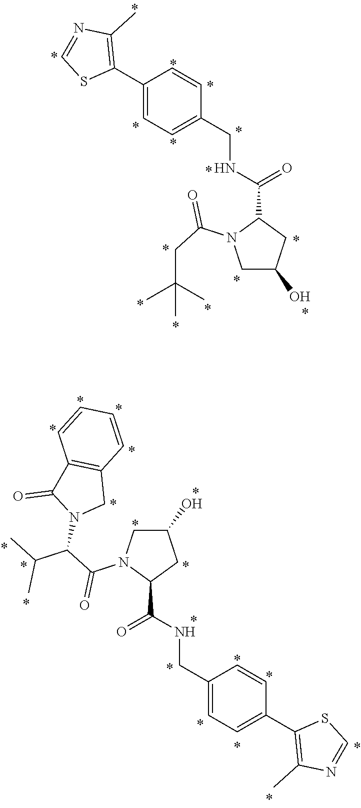

For example, X can have a structure selected from the group consisting of:

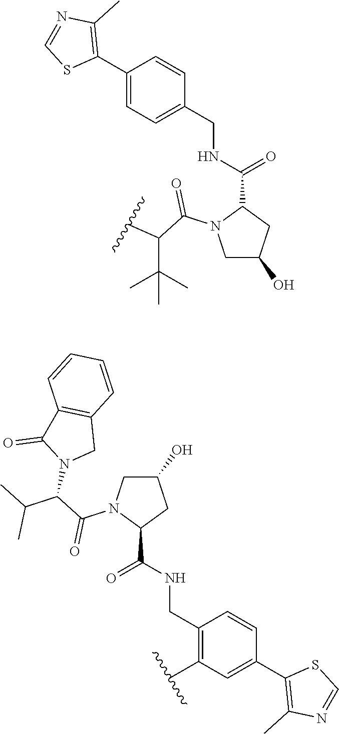

In some cases, X specifically binds to the E3 ubiquitin ligase. As used herein, “specific binding” refers to the ability of a first group (or a first member of a specific binding pair) to preferentially bind to a particular analyte (or a second member of a specific binding pair) in a mixture of different analytes. For example, Y might preferentially bind to the E3 ubiquitin ligase over another protein in a cell, e.g. tumor necrosis factor alpha (TNF-α). In some cases, the binding preference is 5-fold or more, such as 10-fold or more or 100-fold or more.

### Group L

As described above, L is a linking group that is covalently bonded to X and Y. As used herein, “linking group” is used interchangeably with “linker” to refer to a group that connects to two or more other groups, e.g. X and Y. In some cases, the backbone of the linking group L is 100 or less atoms, such as 50 or less or 25 or less. The term “backbone” refers the smallest number of atoms that connect each of the terminal atoms to one another through a sequence of covalent bonds, wherein the terminal atoms are atoms that are bonded to other groups, e.g. X and Y. In some embodiments, L is a hydrocarbon group, e.g. alkyl, alkylene, alkenylene, alkynylene, aryl, heteroaryl, cycloalkyl, heterocycle, or a substituted derivative thereof, or combinations thereof. In some cases the linking group L includes a combination of different groups, e.g., a substituted alkyl connected to a heterocycle.

In some cases, L has a structure of formula (III)

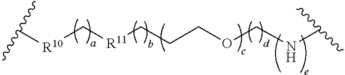

wherein:
a, b, c, and d are each independently an integer from 0 to 6,
e is 0 or 1,
R¹⁰ is absent or has a structure selected from the group consisting of:

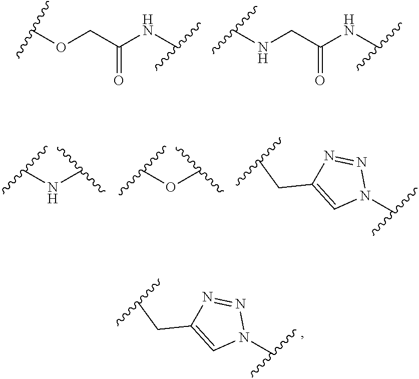

and
R¹¹ is absent or has a structure selected from the group consisting of:

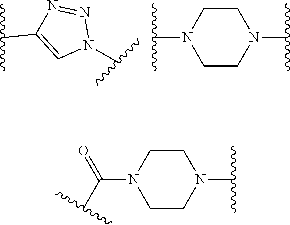

In some cases, a is 1 or 2. In some cases b is 0, 1, or 2. In some cases, c is 0, 1, 2, or 3. In some cases d is 0. In some cases e is 0. In some cases a is 1 or 2, b is 0, 1, or 2, c is 0, 1, 2, or 3,d is 0, and e is 0.

In some cases, R¹¹ is absent and R¹⁰ has a structure selected from the group consisting of:

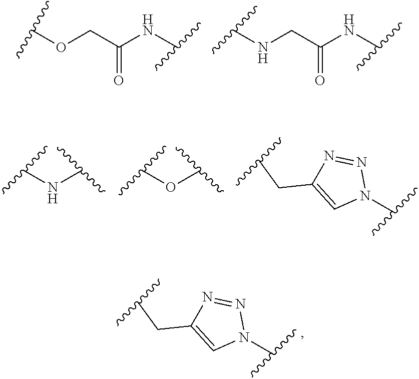

In some cases, R¹⁰ is absent and R¹¹ has a structure selected from the group consisting of:

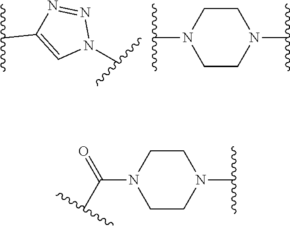

In some cases, L has a structure selected from the linking groups of the compounds discussed in the Specific Embodiments section below. For example, Compound 1.0 is discussed in the Specific Embodiments section and also shown below.

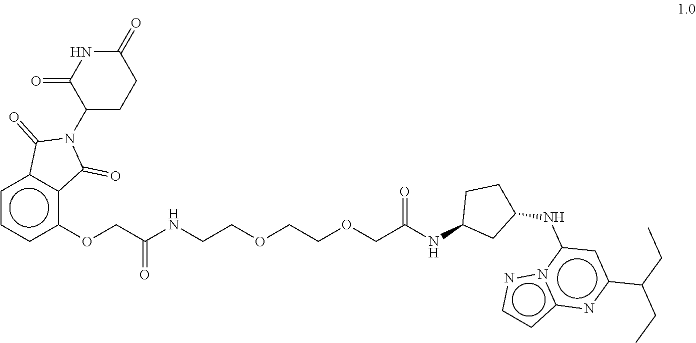

It can be seen that Compound 1.0 has an X group and Y group that are discussed above. Hence, it can be shown that the middle L group has the structure shown below.

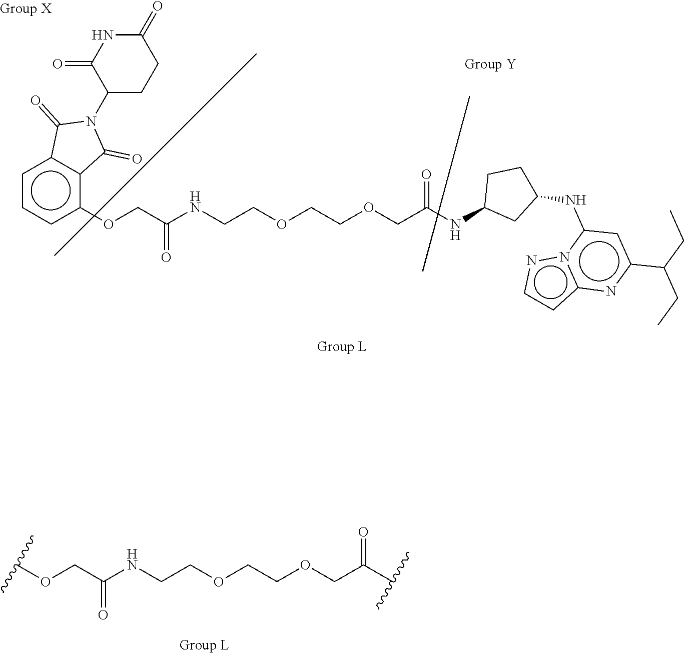

This analysis can be repeated with each of the compounds of the Specific Embodiments section below and demonstrates several different identities for the L linker.

Any other suitable linking group, i.e. linker, can be used. In some cases, the linking group includes a alkyl chain, aryl chain, or polyethylene glycol chain that is terminated in its two ends with two coupling group each independently selected from an amine, carbamate, carboxylic acid, carboxylate, maleimide, activated ester, N-hydroxysuccinimidyl, hydrazine, hydrazide, hydrazine, azide, alkyne, aldehyde, or thiol. In some cases, the two coupling groups are different from one another, allowing for selective coupling of the first end of the linker to the X group and the selective coupling of the second end of the linker to the Y group. Exemplary methods of forming the compounds is shown in the Examples section.

Exemplary linking strategies include those used in the field of antibody-drug conjugates. For instance, Tsuchikama and An describe conjugation and linking chemistries, which is incorporated herein by reference (Protein Cell, 2018, 9(1):33, doi:10.1007/s13238-016-0323-0). Additional exemplary linkers are described in U.S. Pat. Nos. 9,872,924 and 10,377,837; PCT Publication WO 2020/005754; and US Patent Publication 2019/0388555, which are incorporated herein by reference.

### Specific Embodiments

In some cases, the compound has a structure selected from the group consisting of:

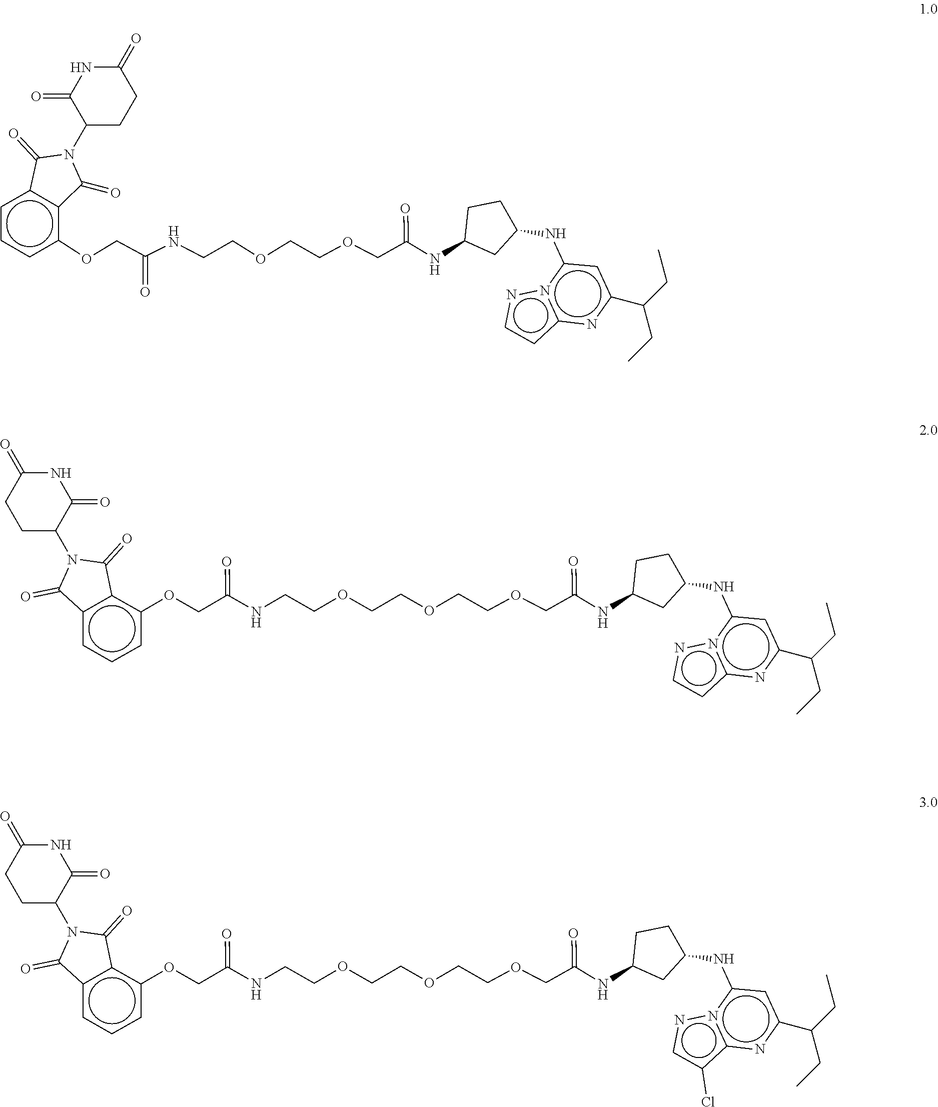

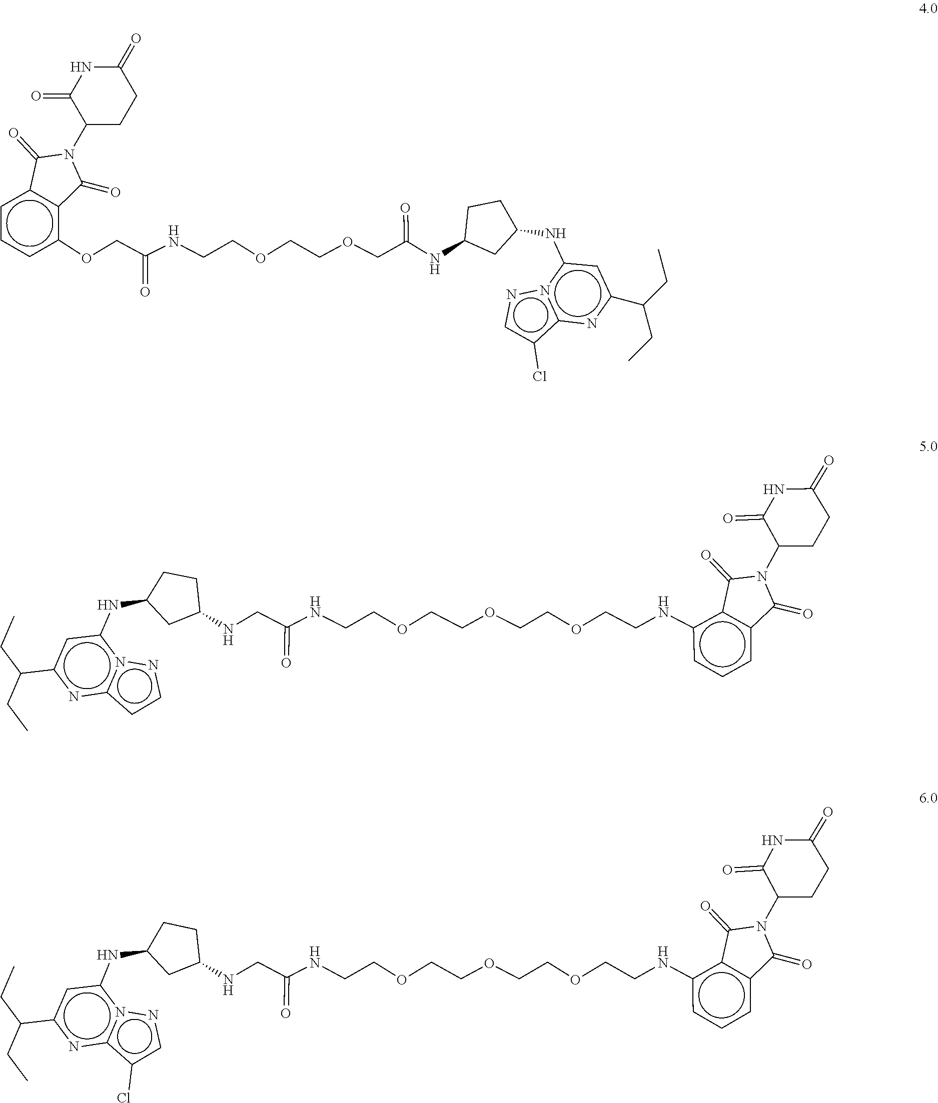

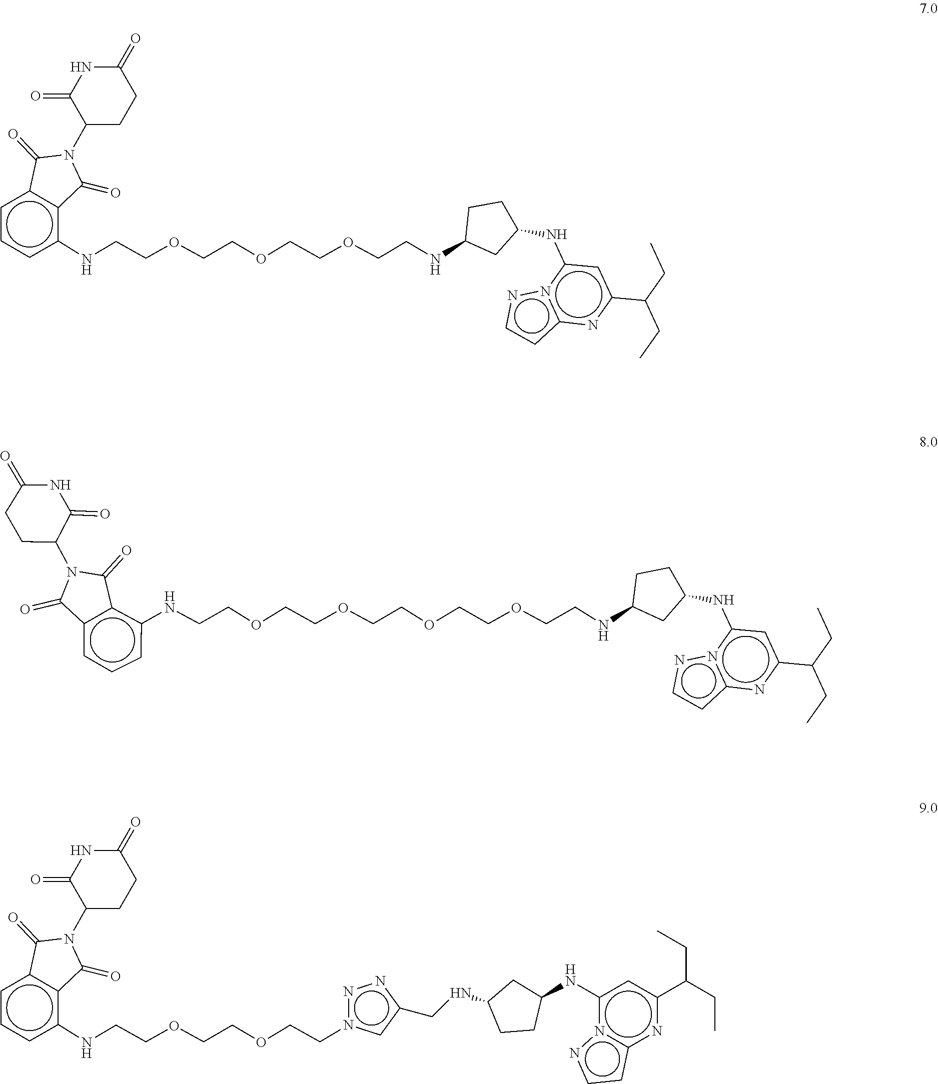

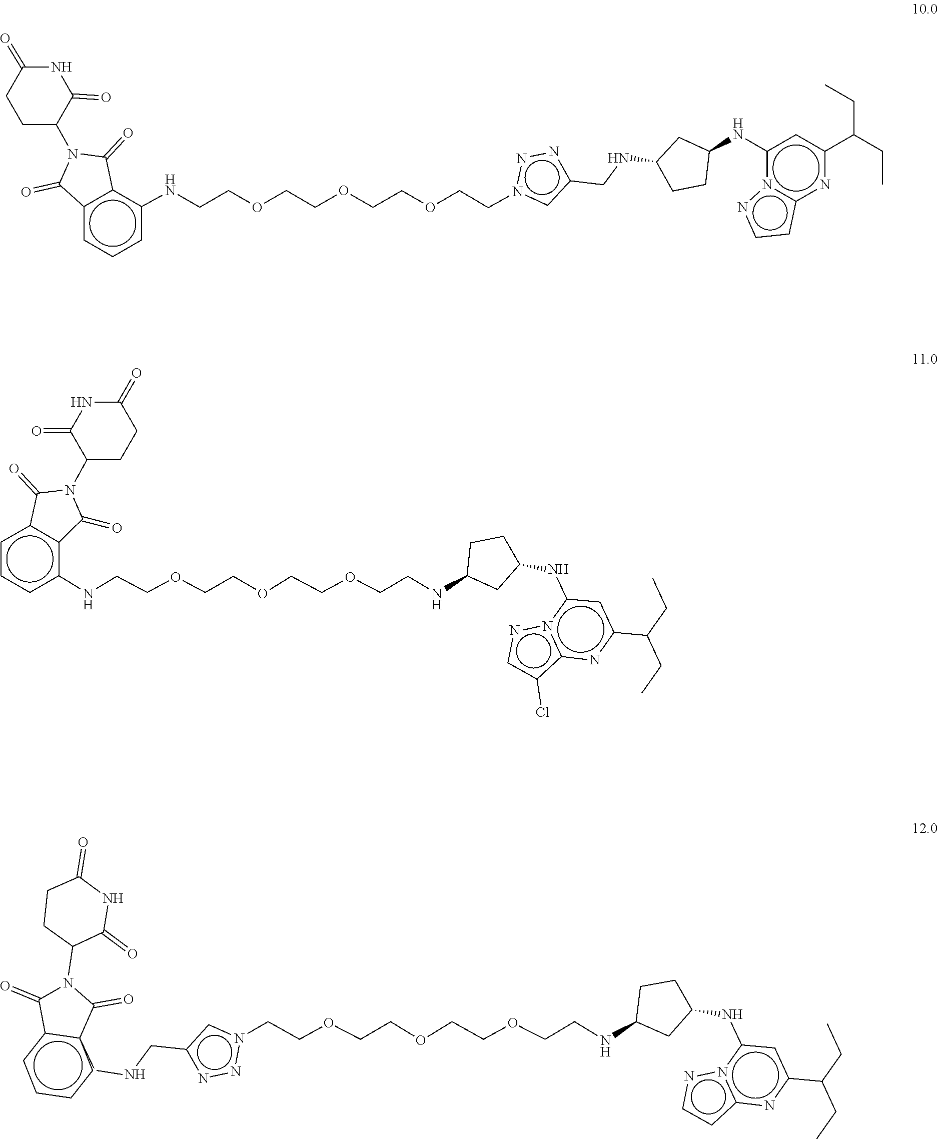

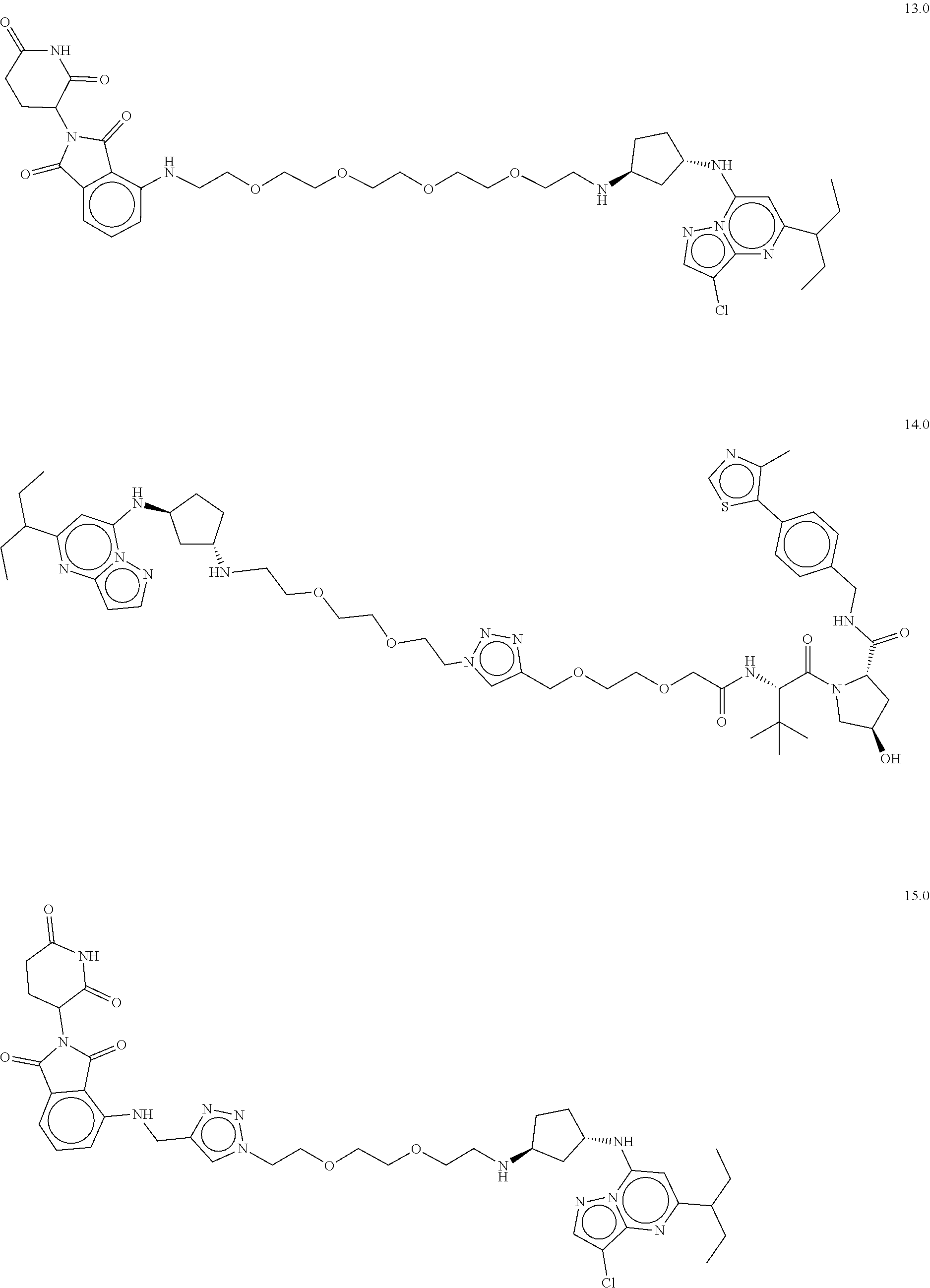

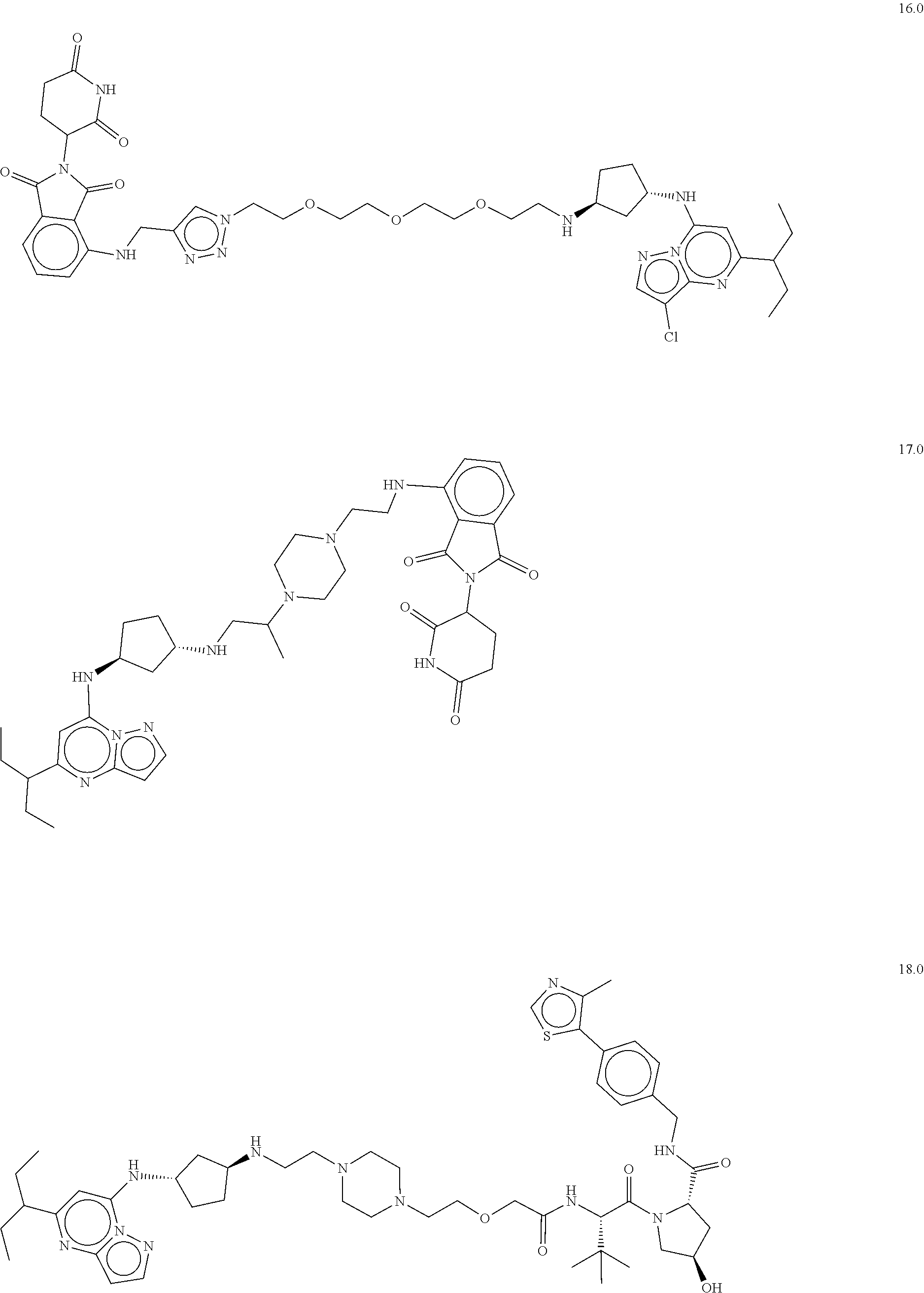

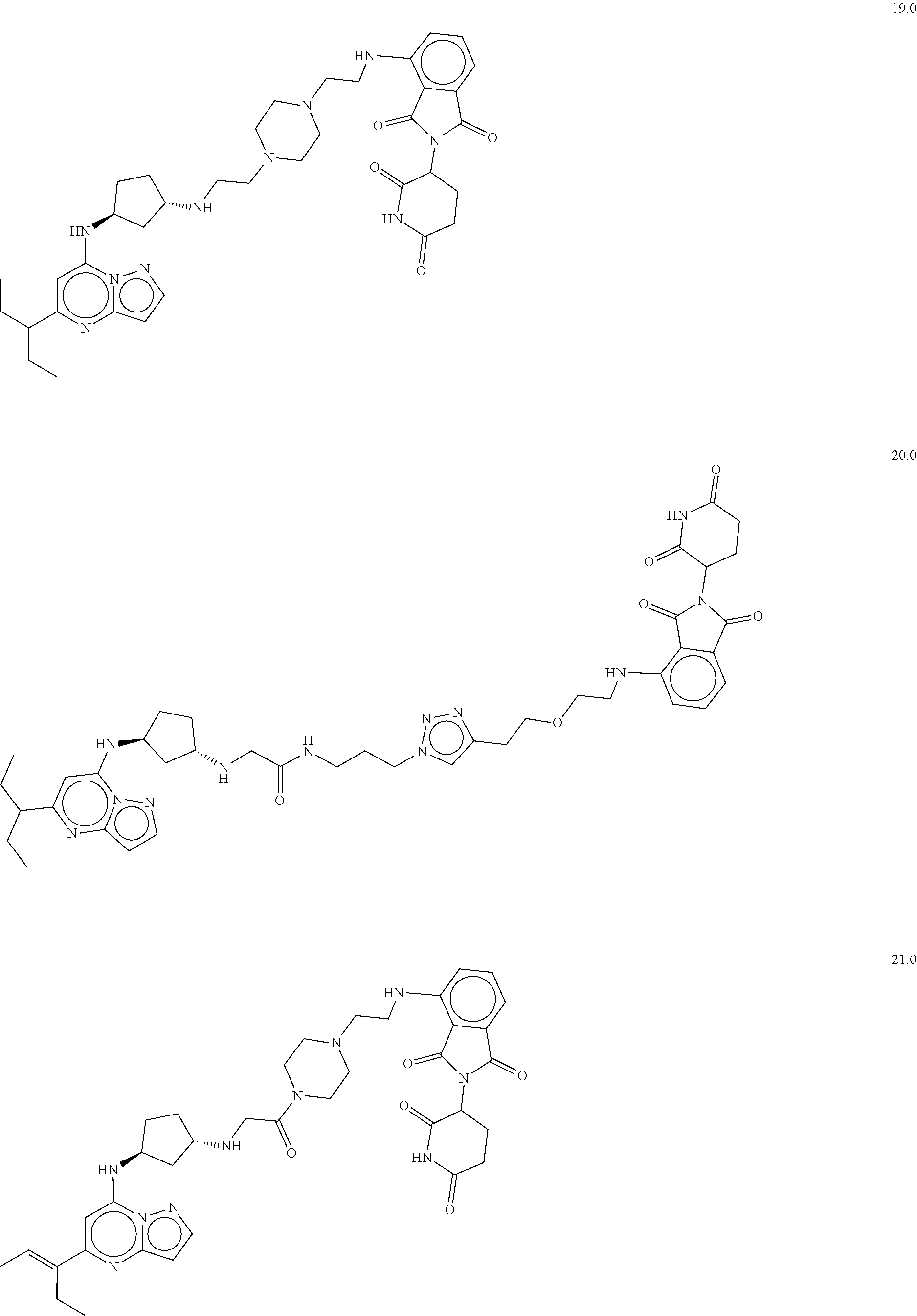

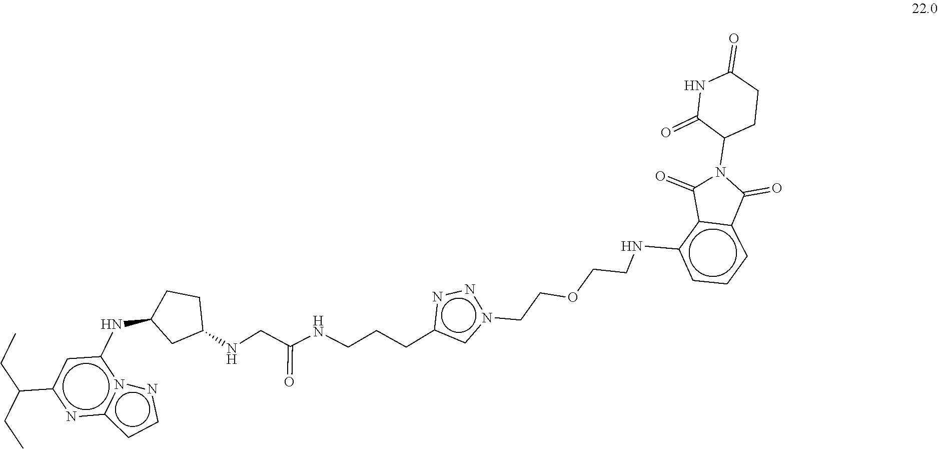

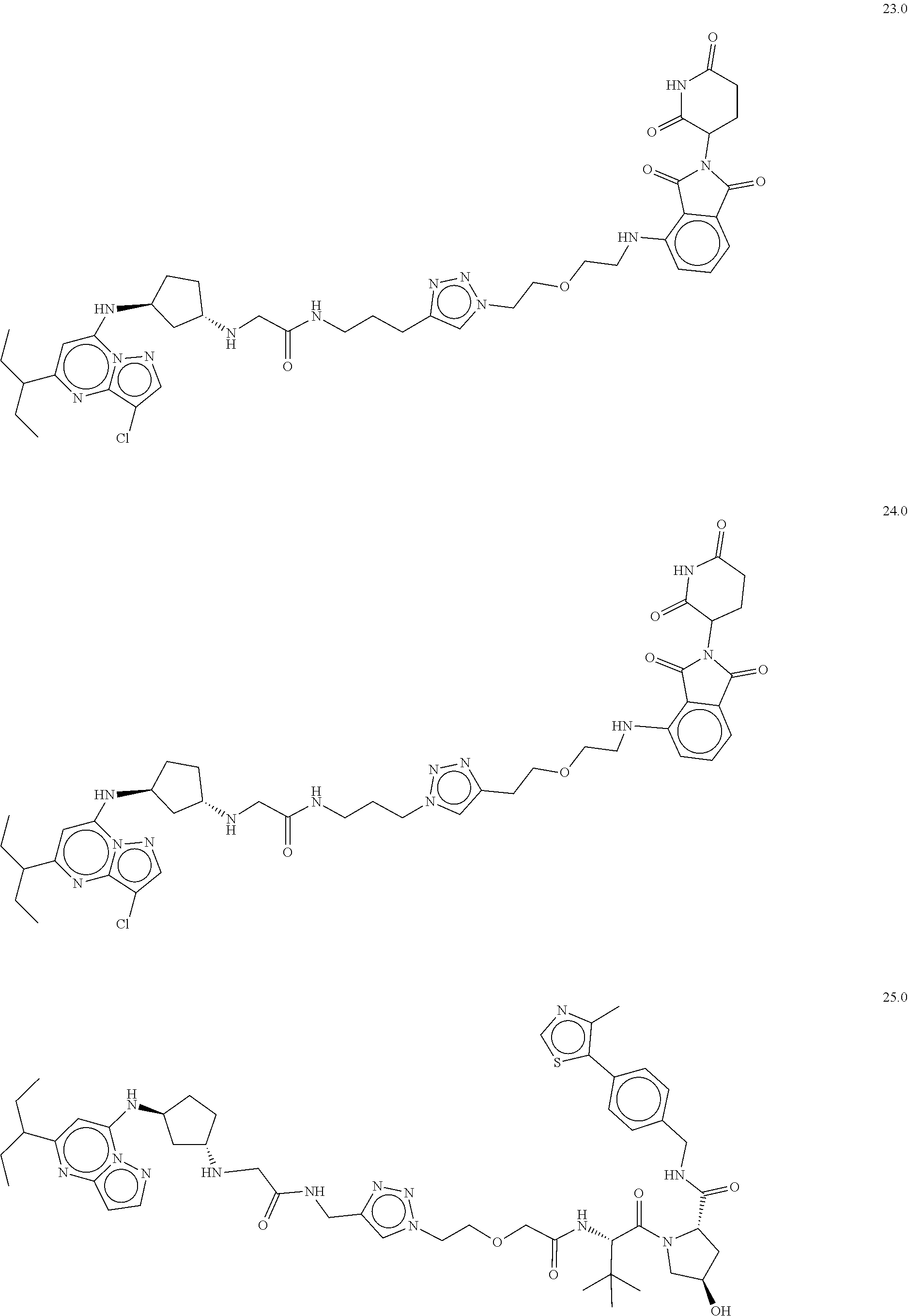

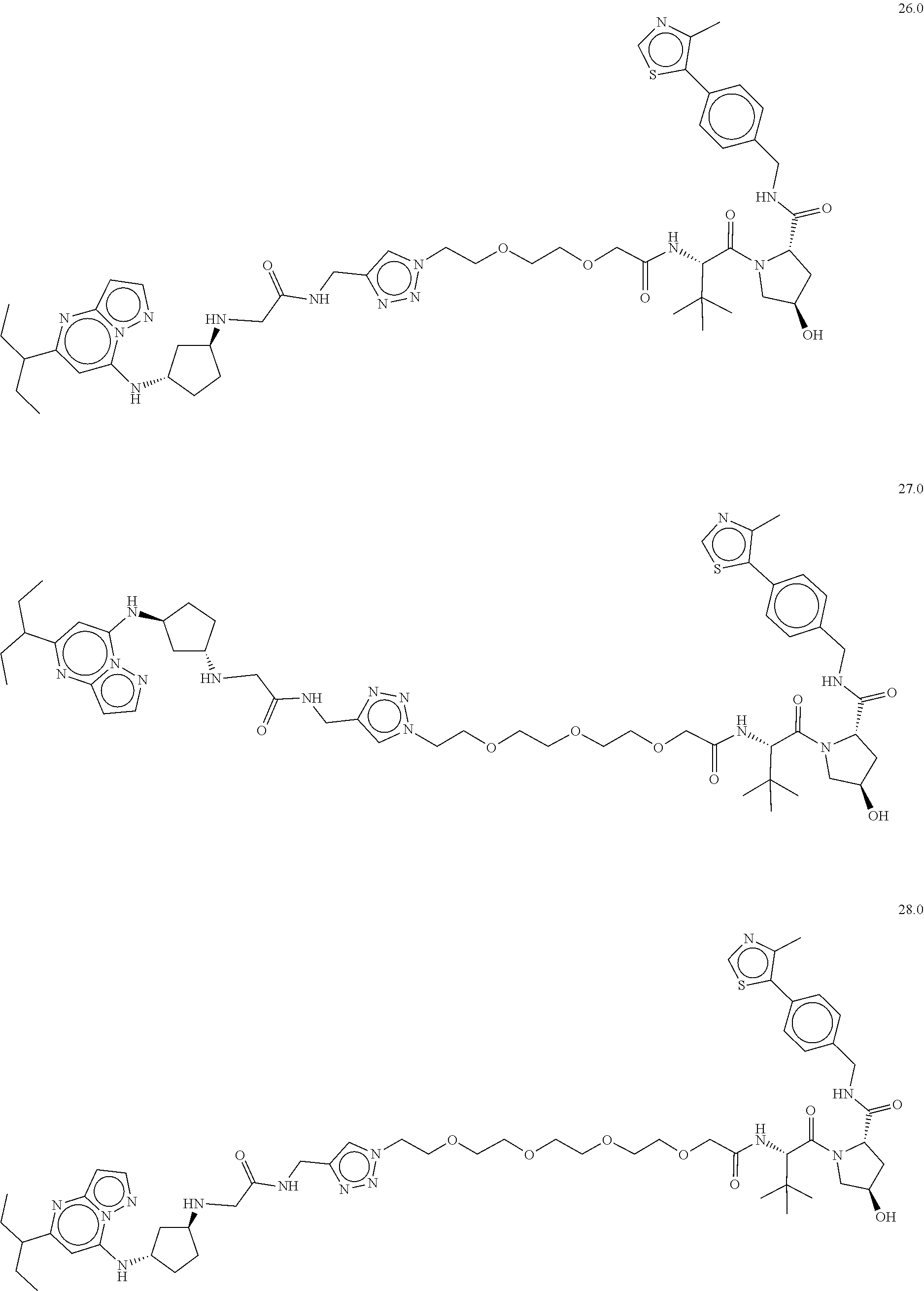

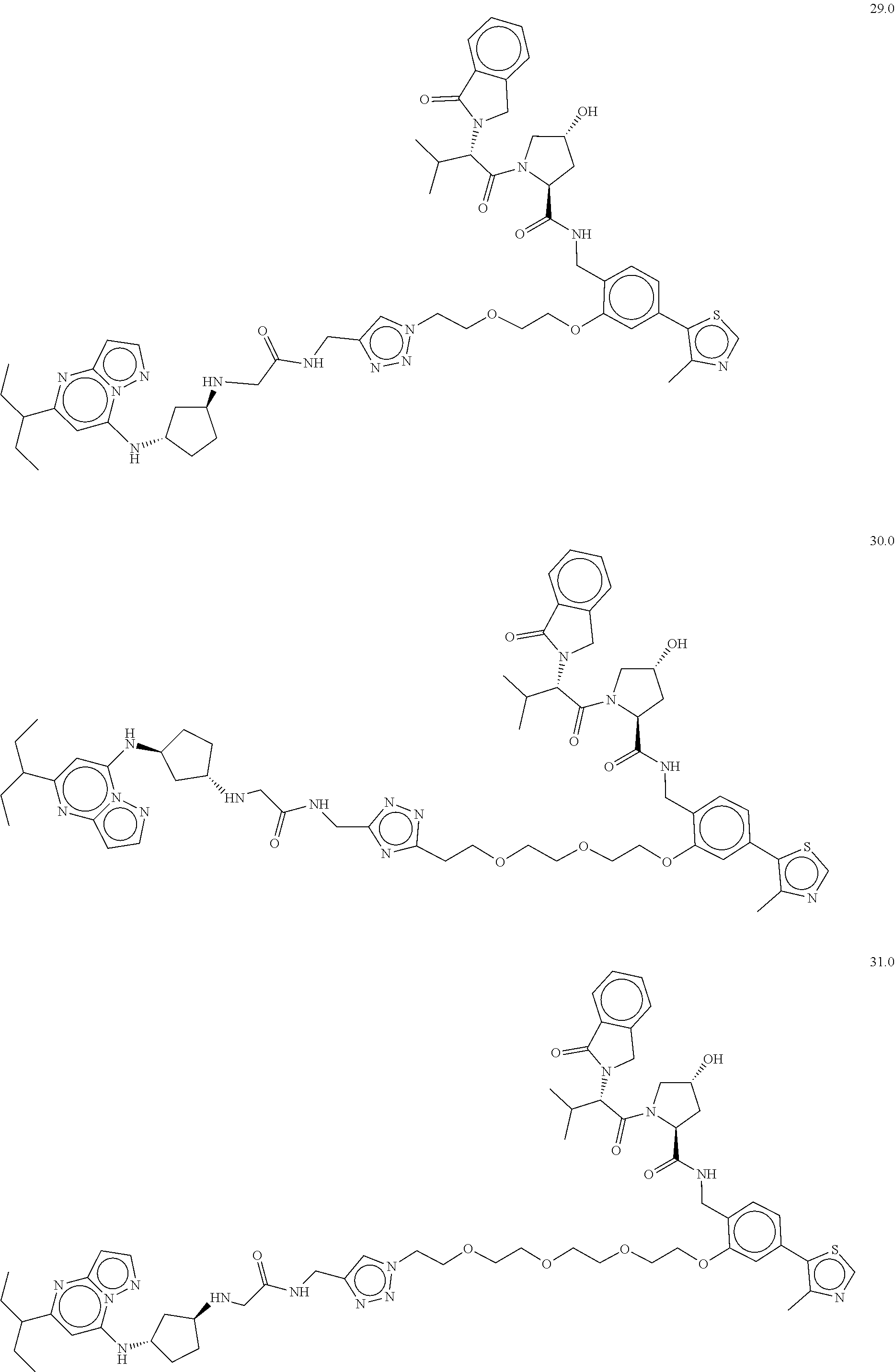

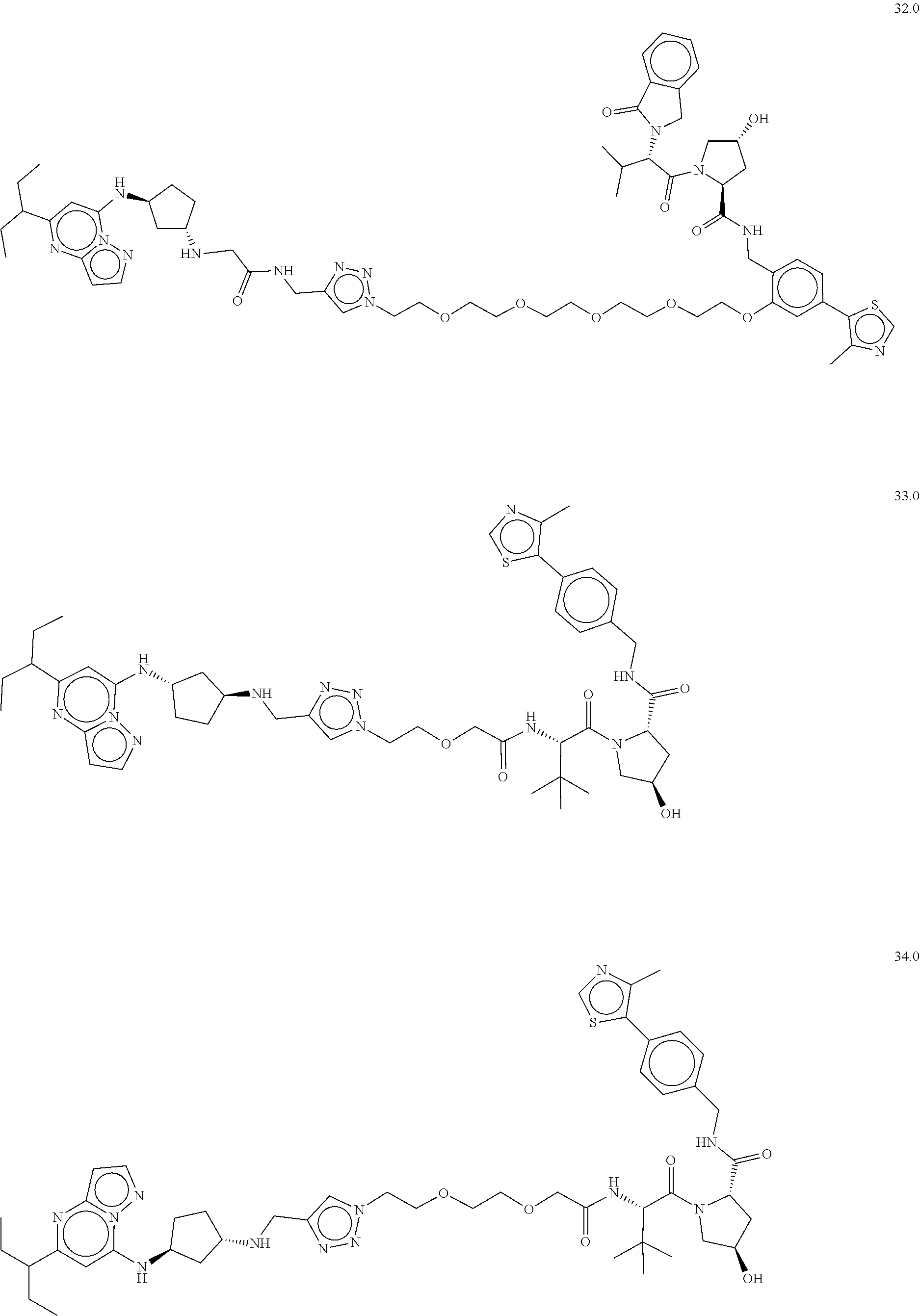

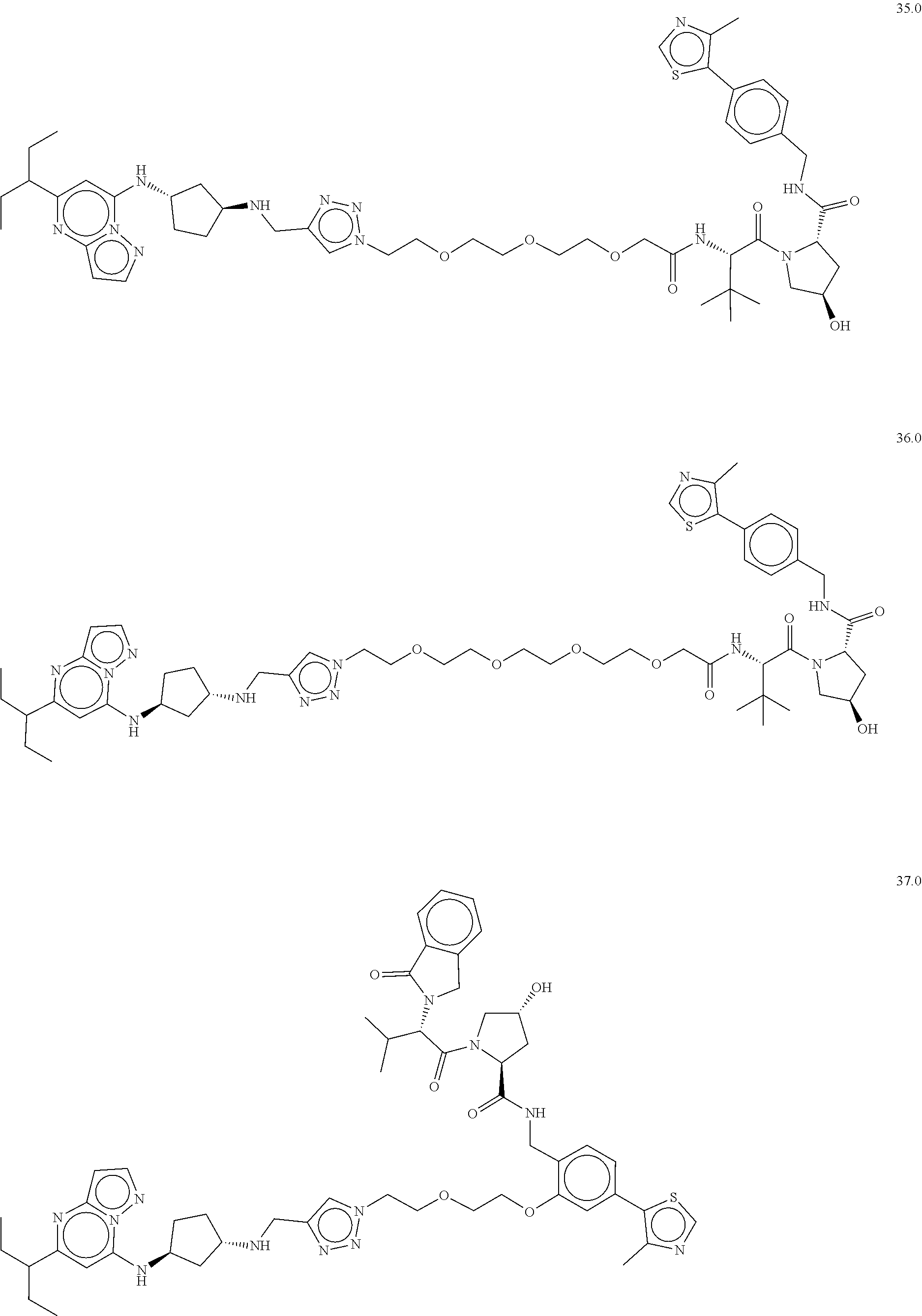

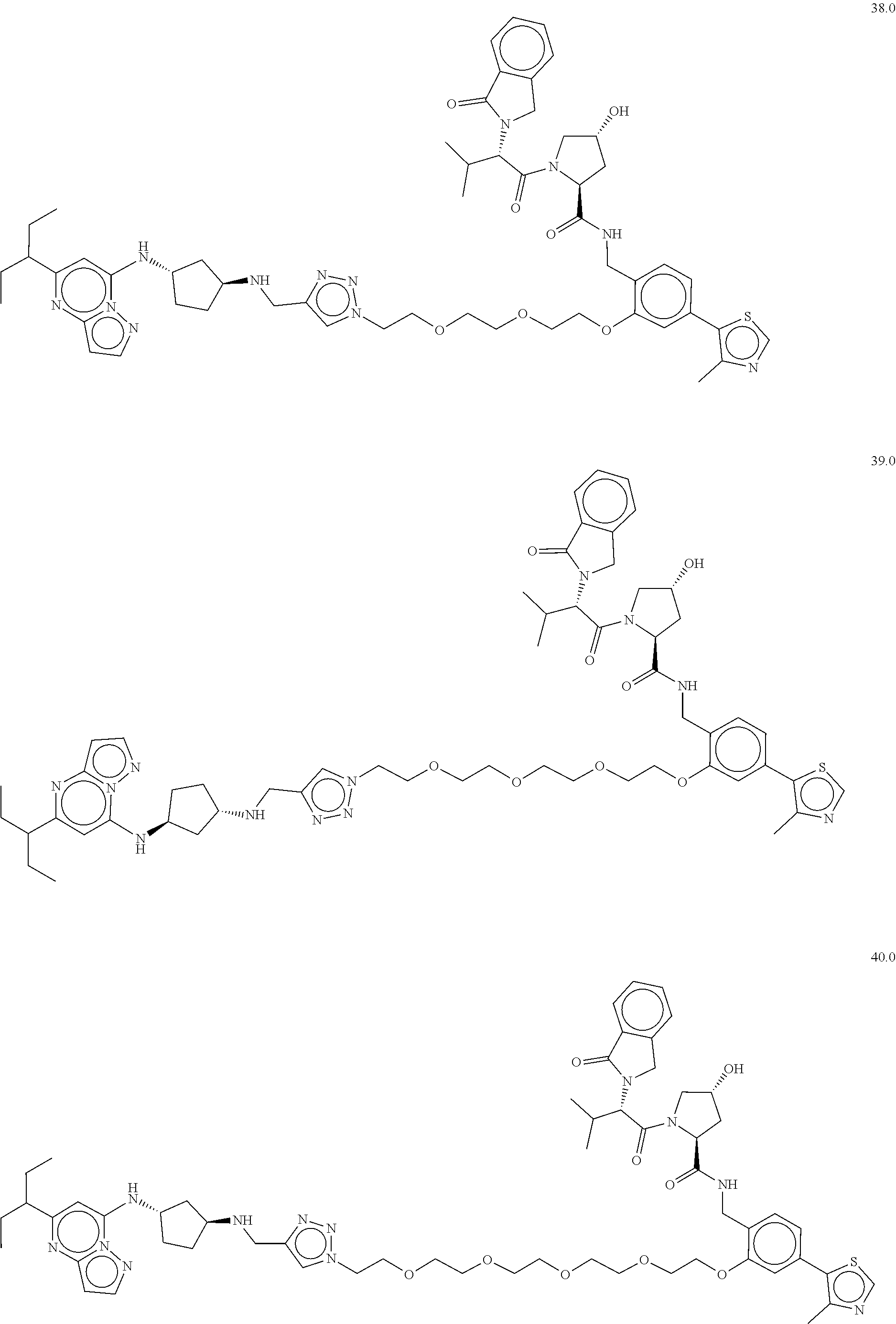

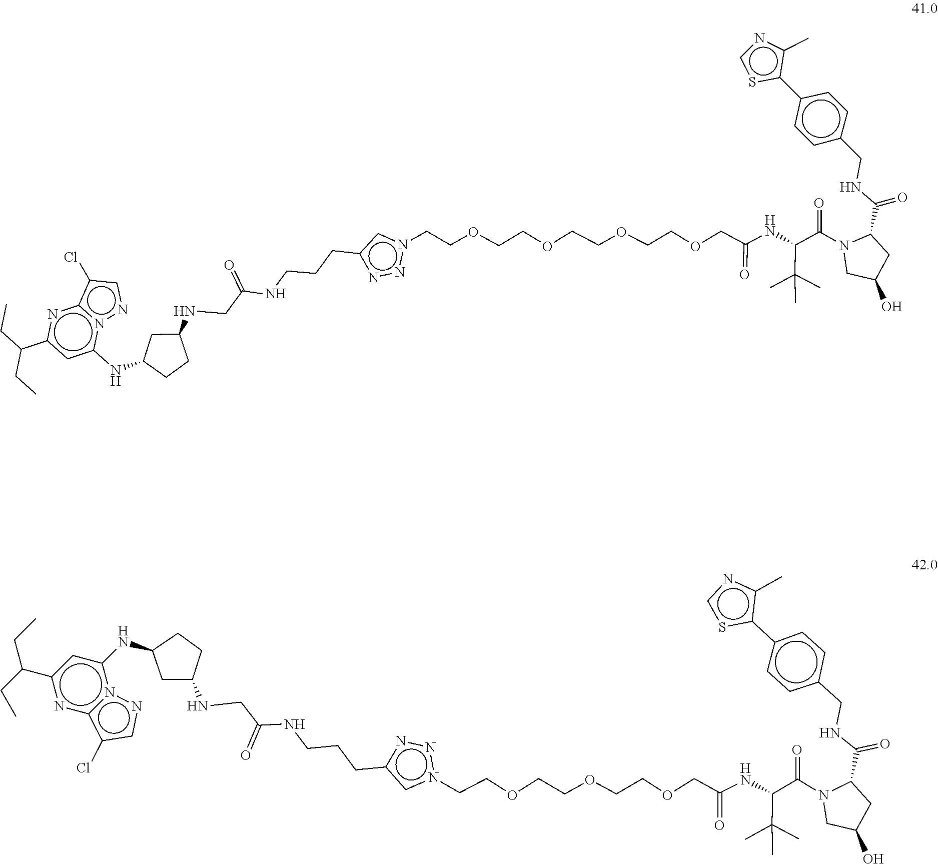

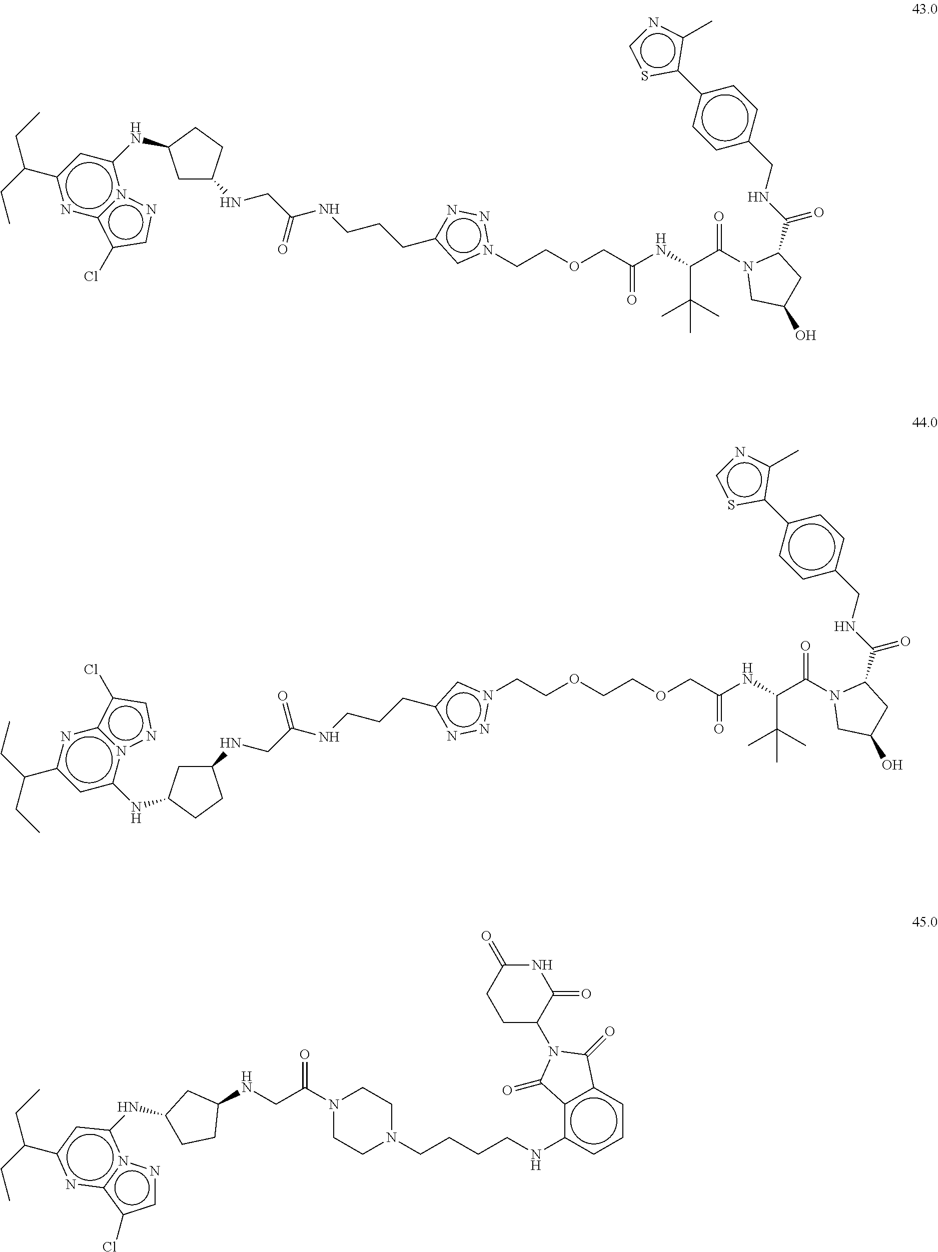

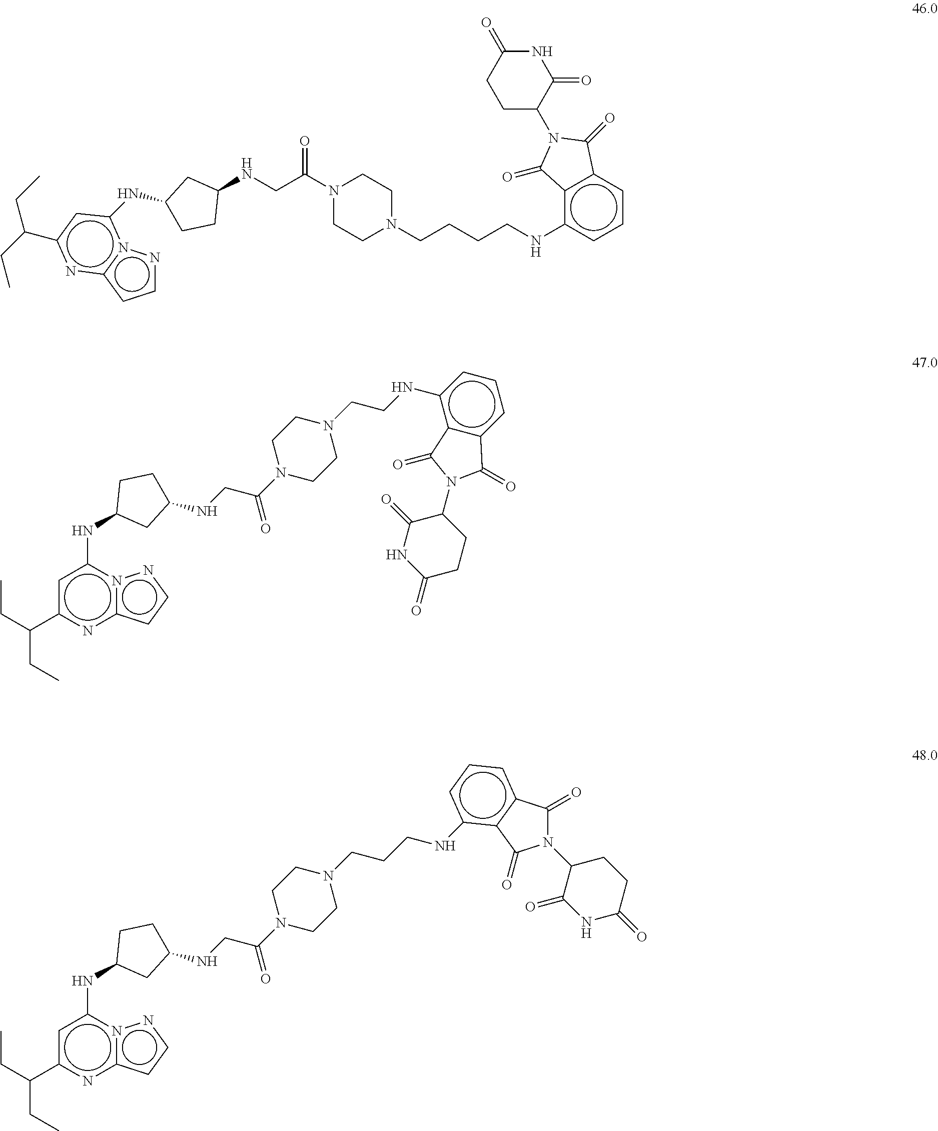

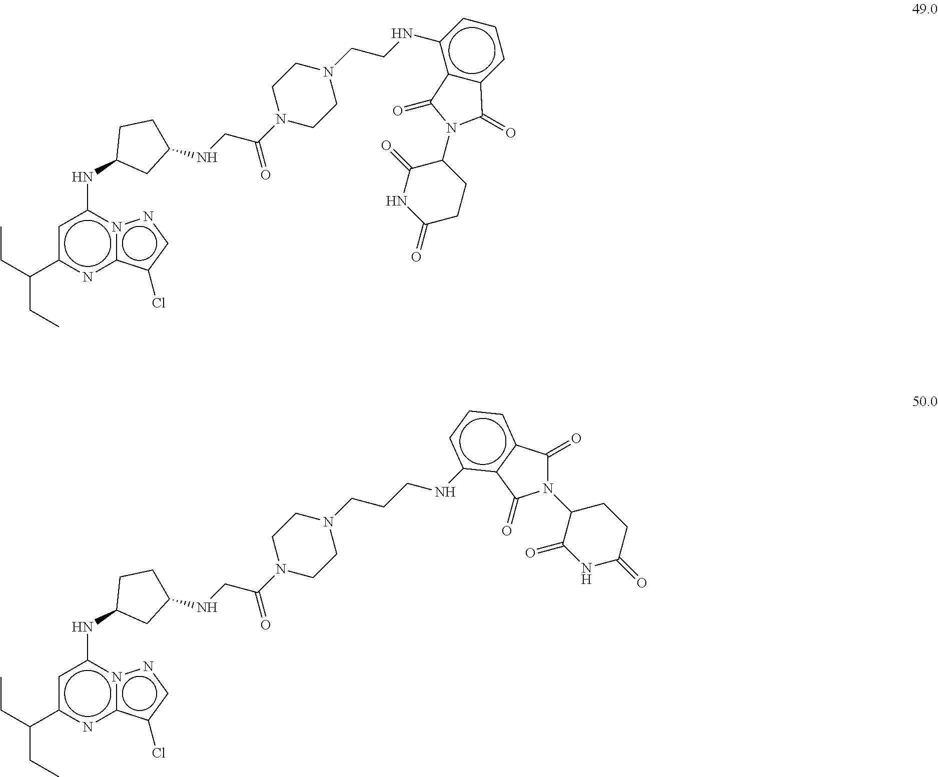

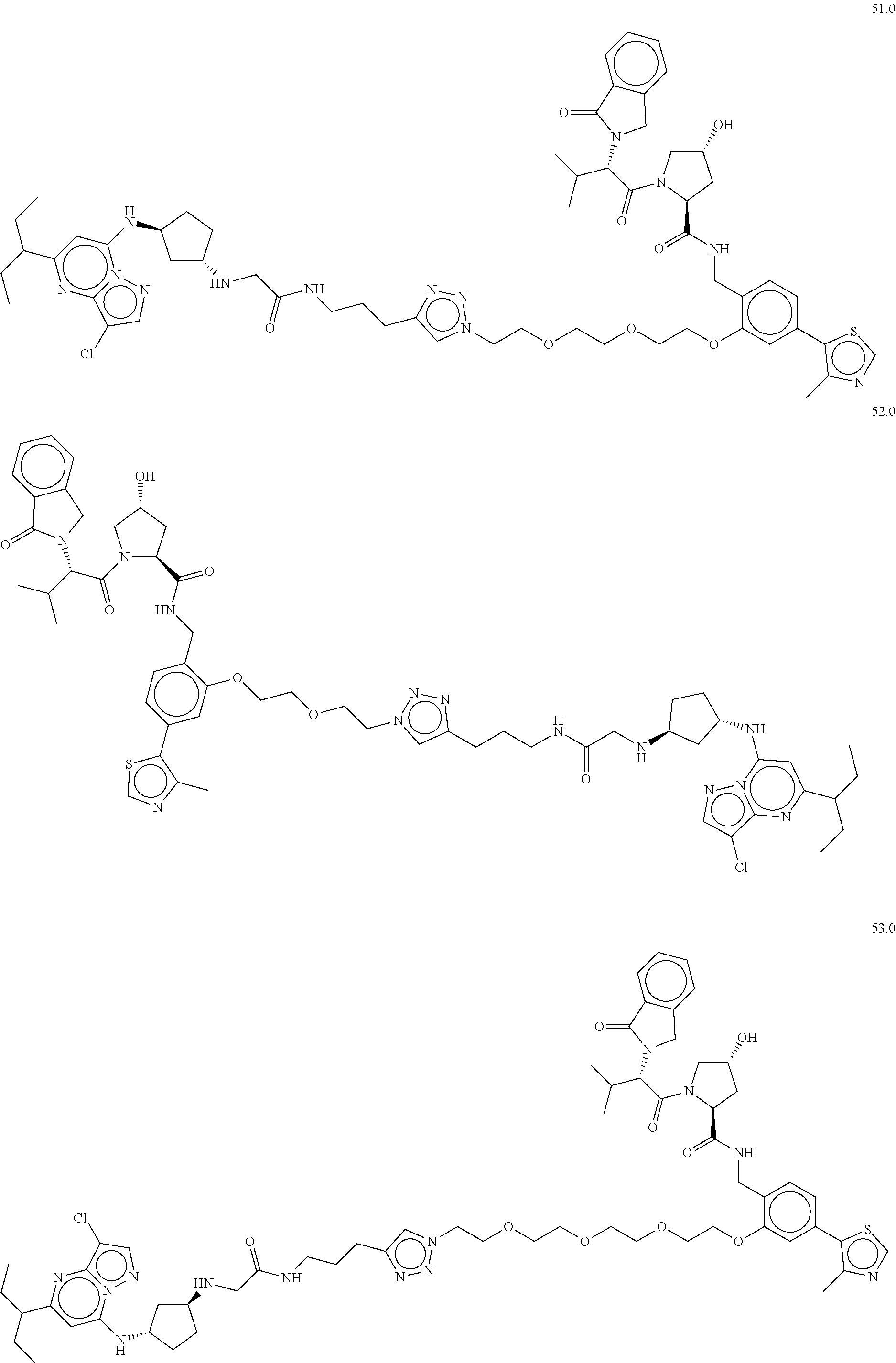

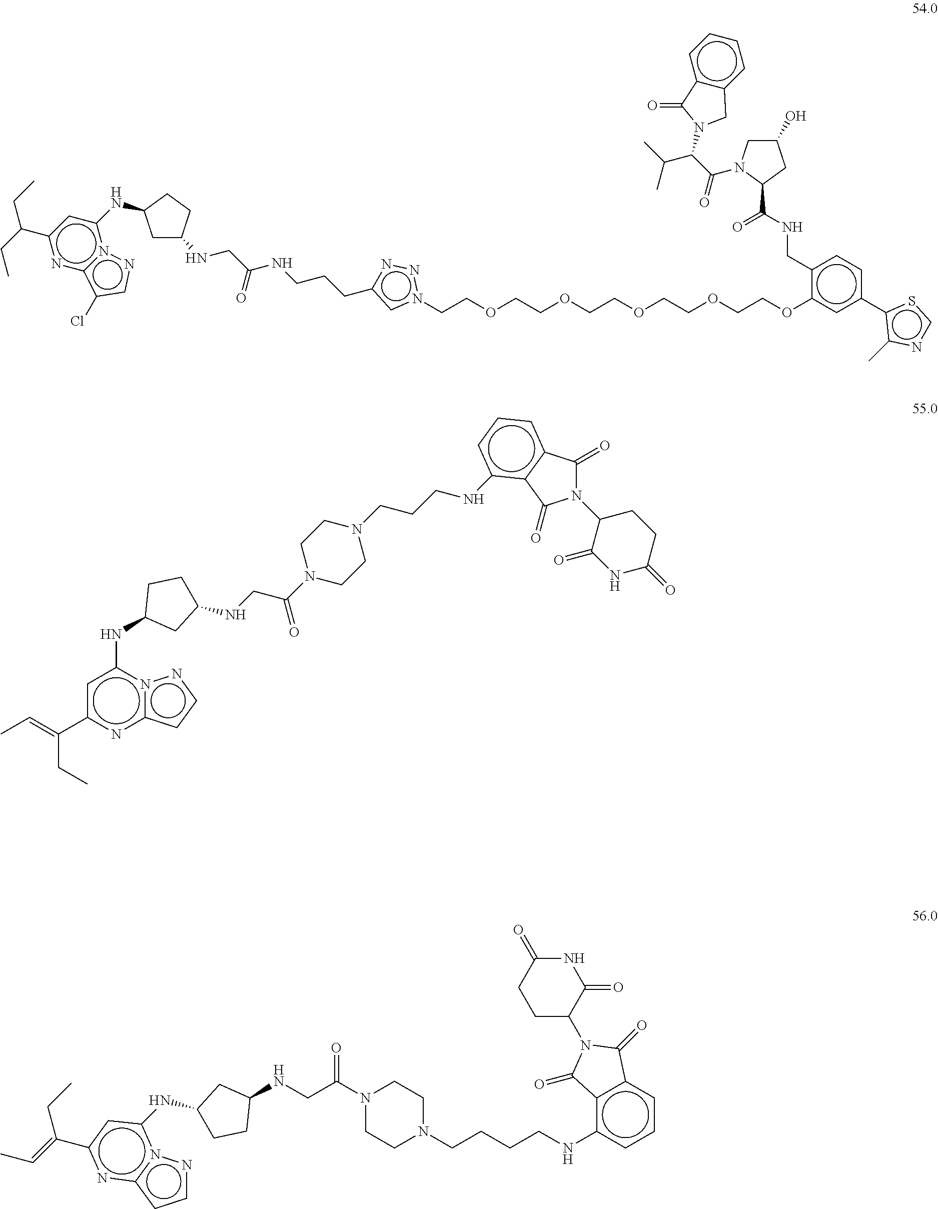

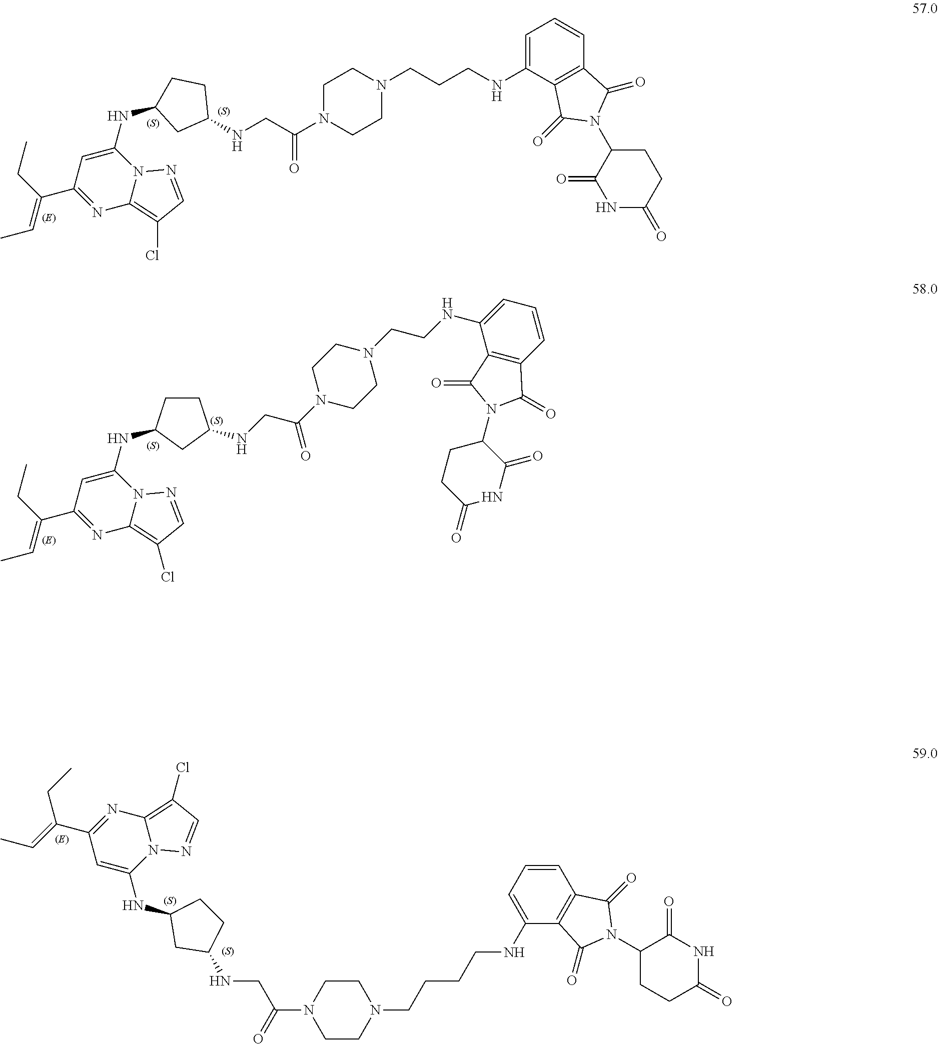

### Methods

Also provided are methods of treating a subject for a CDK9-mediated disease by administering a therapeutically effective dose of a pharmaceutical composition including a heterobifunctional degrader compound described above.

In some cases, the CDK9-mediated disease is cancer, e.g. cancer caused by aberrant expression of MYC- or MCL-1, a hematologic malignancy, or a solid tumor. In some cases, the disease is selected from the group consisting of: acute myelogenous leukemia, primary peritoneal carcinoma, chronic lymphocytic leukemia, relapsed multiple myeloma, non-Hodgkin's lymphoma, acute lymphoblastic leukemia, acute byphenotypic leukemia, advanced breast cancer, non-small cell lung cancer, and liver cancer.

In some cases, the compound is a compound described in the Specific Embodiments section above.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); nt, nucleotide(s); and the like. The term “RT” refers to room temperature.

### Compound 1.0 2-[2-[2-[[2-[2-(2,6-dioxo-3-piperidyl)-1,3-dioxo-isoindolin-4 yl]oxyacetyl]amino]ethoxy]ethoxy]-N-[(1S,3S)-3-[[5-(1-ethylpropyl)pyrazolo[1,5-a]pyrimidin-7-yl]amino]cyclopentyl]acetamide

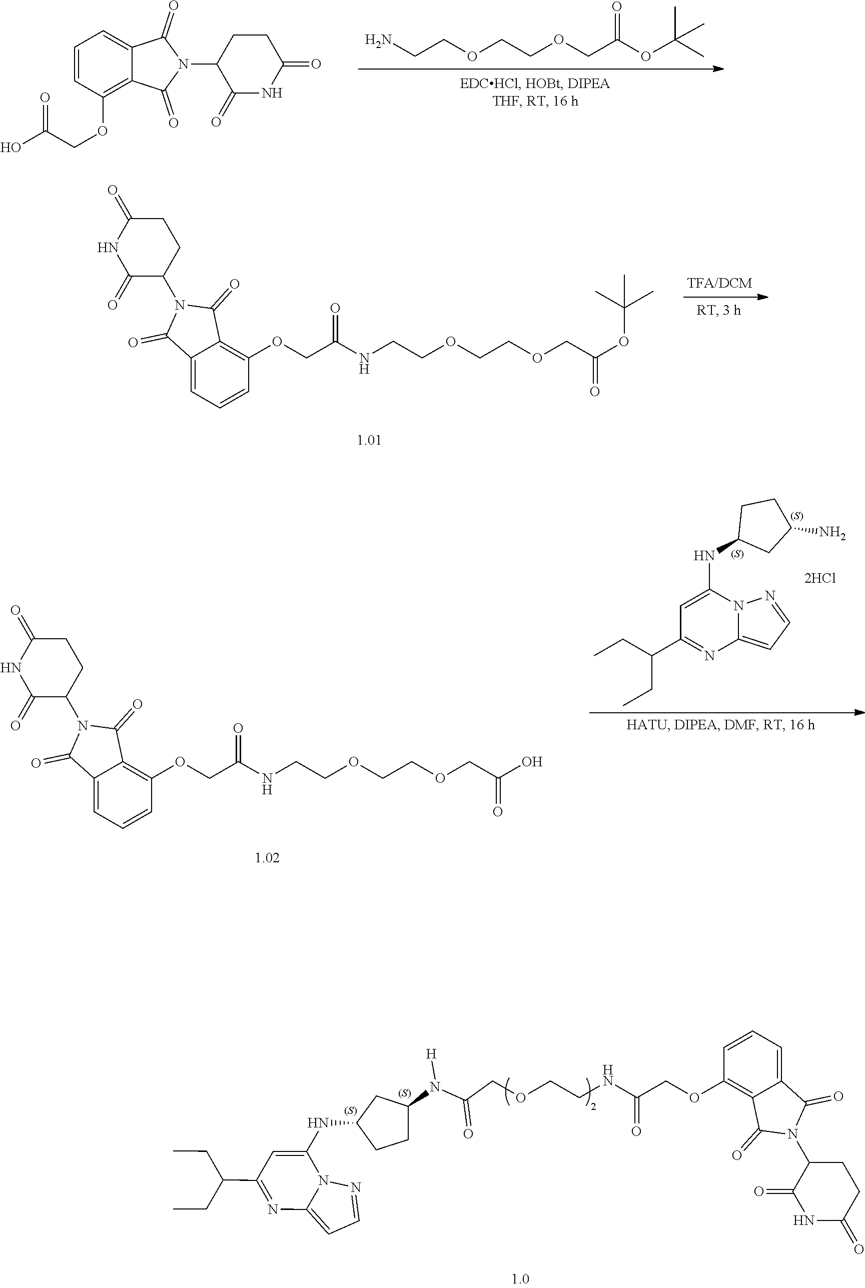

### Synthesis of tert-butyl 2-[2-[2-[[2-[2-(2,6-dioxo-3-piperidyl)-1,3-dioxo-isoindolin-4-yl]oxyacetyl]amino]ethoxy]ethoxy]acetate (Compound 1.01)

To a stirred solution of DIPEA (0.4 mL, 2.26 mmol) dissolved in THF (5 mL), under nitrogen atmosphere were added HOBt (91.42 mg, 0.6800 mmol), EDC·HCl (129.34 mg, 0.6800 mmol), and 2-[2-(2,6-dioxo-3-piperidyl)-1,3-dioxo-isoindolin-4-yl]oxyacetic acid (150 mg, 0.4500 mmol) at 0° C. The reaction was stirred for 10 min at same temperature and then tert-butyl 2-[2-(2-aminoethoxy)ethoxy]acetate hydrochloride (115.45 mg, 0.4500 mmol) was added, then the reaction mixture was allowed to stir at ambient temperature for 16 h. The progress of the reaction was monitored by TLC (5% MeOH in DCM, Product Rf=0.7, SM Rf=0.2) and LCMS. The reaction mixture was quenched with ice cold water, concentrated under reduced pressure, extracted with EtOAc (50 mL×1), and organic layer was washed with saturated brine solution (50 mL×1). The organic layer was dried over anhydrous Na₂SO₄, and solvent removed and purified by Combiflash column chromatography (silica gel), eluent 5% MeOH in DCM to give tert-butyl 2-[2-[2-[[2-[2-(2,6-dioxo-3-piperidyl)-1,3-dioxo-isoindolin-4-yl]oxyacetyl]amino]ethoxy]ethoxy]acetate (Compound 1.01) (100 mg, 42%) as a sticky solid. LCMS-ESI (pos.) m/z: 534.1 (M+1)⁺

### Synthesis of 2-[2-[2-[[2-[2-(2,6-dioxo-3-piperidyl)-1,3-dioxo-isoindolin-4 yl]oxyacetyl]amino]ethoxy]ethoxy]acetic acid (Compound 1.02)

To a stirred solution of tert-butyl 2-[2-[2-[[2-[2-(2,6-dioxo-3-piperidyl)-1,3-dioxo-isoindolin-4-yl]oxyacetyl]amino]ethoxy]ethoxy]acetate (100 mg, 0.1900 mmol) (Compound 1.01) in DCM (1.0 mL) at 0° C. was added TFA (0.72 mL, 9.37 mmol). The reaction was allowed to warm to room temperature and continuously stirred for 3 h. After completion of reaction, monitored by LCMS, TFA was removed under reduced pressure and the crude was triturated with n-pentane to give 2-[2-[2-[[2-[2-(2,6-dioxo-3-piperidyl)-1,3-dioxo-isoindolin-4 yl]oxyacetyl]amino]ethoxy]ethoxy]acetic acid (Compound 1.02) (60 mg, 67%) as an off-white solid. LCMS-ESI (pos.) m/z: 478.4 (M+1)⁺

### Synthesis of 2-[2-[2-[[2-[2-(2,6-dioxo-3-piperidyl)-1,3-dioxo-isoindolin-4 yl]oxyacetyl]amino]ethoxy]ethoxy]—N-[(1S,3S)-3-[[5-(1-ethylpropyl)pyrazolo[1,5-a]pyrimidin-7-yl]amino]cyclopentyl]acetamide (Compound 1.0)

To a stirred solution of (1S,3S)—N3-[5-(1-ethylpropyl)pyrazolo[1,5-a]pyrimidin-7-yl]cyclopentane-1,3-diamine hydrochloride (36.04 mg, 0.1100 mmol) (CAS #2416873-83-9, prepared using the procedure as described in US20200131189 A1) in DMF (0.2893 mL), was added DIPEA (0.35 mL, 2.49 mmol) at 0° C. Then 2-[2-[2-[[2-[2-(2,6-dioxo-3-piperidyl)-1,3-dioxo-isoindolin-4-yl]oxyacetyl]amino]ethoxy]ethoxy]acetic acid (50 mg, 0.1000 mmol) (Compound 1.02) and HATU (568.19 mg, 1.49 mmol) were added. The reaction was stirred at room temperature for 16 h, the progress of the reaction was monitored by TLC (5% MeOH in DCM, Product Rf=0.7, SM Rf=0.4) and LCMS. The reaction mixture was quenched with ice cold water and extracted with EtOAc (30 mL×2), and organic layer was washed with saturated brine solution (30 mL×1). The organic layer was dried over anhydrous Na₂SO₄, and solvent concentrated and purified by Prep HPLC to give 2-[2-[2-[[2-[2-(2,6-dioxo-3-piperidyl)-1,3-dioxo-isoindolin-4-yl]oxyacetyl]amino]ethoxy]ethoxy]-N-[(1S,3S)-3-[[5-(1-ethylpropyl)pyrazolo[1,5-a]pyrimidin-7-yl]amino]cyclopentyl]acetamide (Compound 1.0) (28 mg, 36%) as an off-white solid. LCMS-ESI (pos.) m/z: 747.8 (M+1)⁺

¹H NMR (400 MHz, DMSO-d₆) δ 11.11 (s, 1H), 8.05-7.97 (m, 2H), 7.80 (t, J=7.9 Hz, 1H), 7.71-7.57 (m, 2H), 7.49 (d, J=7.5 Hz, 1H), 7.40 (d, J=8.7 Hz, 1H), 6.31 (s, 1H), 5.96 (s, 1H), 5.11 (d, J=9.8 Hz, 1H), 4.79 (s, 2H), 4.36-4.27 (m, 2H), 4.24-4.18 (m, 2H), 3.87 (s, 2H), 3.59 (s, 4H), 3.53-3.47 (m, 2H), 2.95-2.85 (m, 3H), 2.22-2.12 (m, 3H), 2.03 (s, 3H), 1.79-1.52 (m, 6H), 0.76 (t, J=7.3 Hz, 6H).

### Compound 2.0 2-[2-[2-[2-[[2-[2-(2,6-dioxo-3-piperidyl)-1,3-dioxo-isoindolin-4-yl]oxyacetyl]amino]ethoxy]ethoxy]ethoxy]-N-[(1S,3S)-3-[[5-(1-ethylpropyl)pyrazolo[1,5-a]pyrimidin-7-yl]amino]cyclopentyl]acetamide

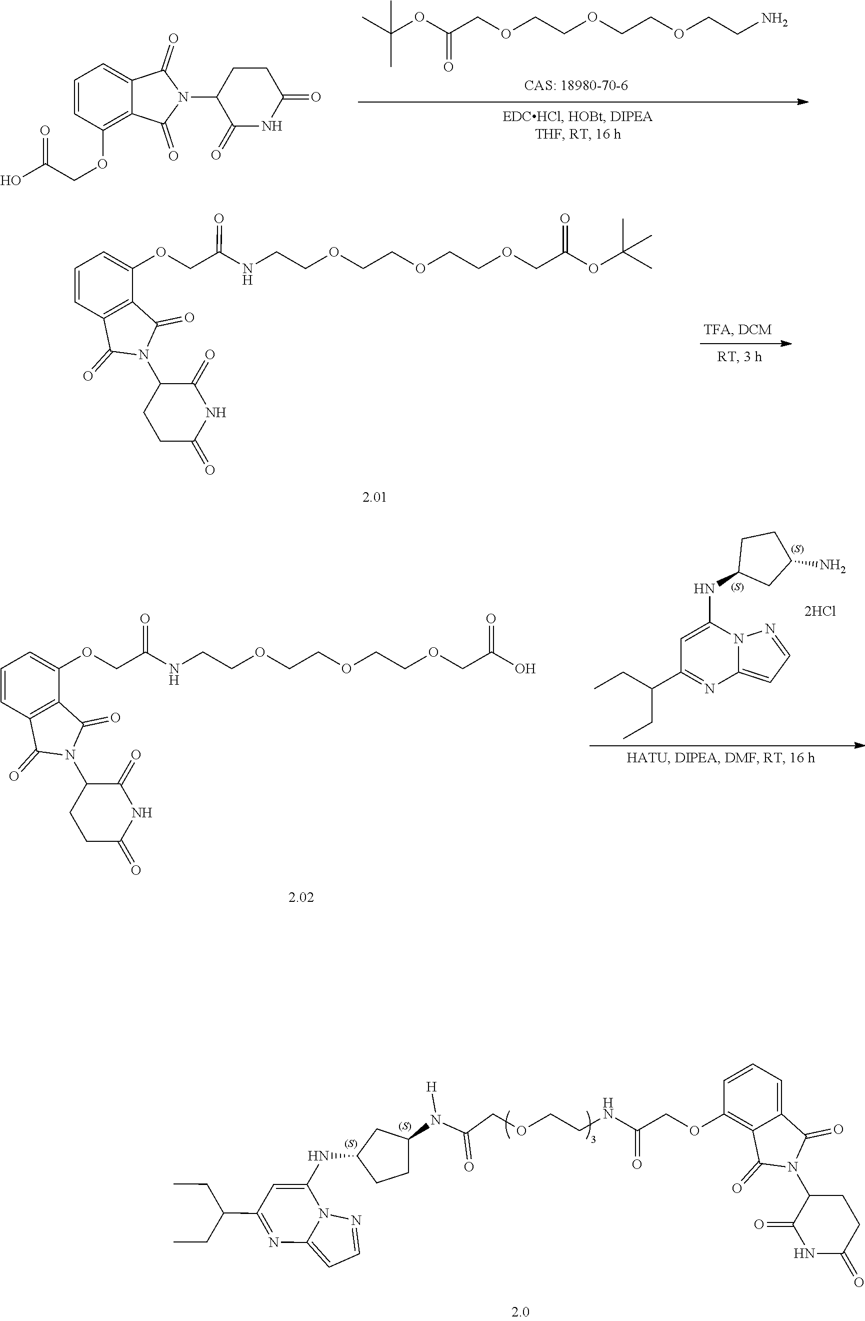

### Synthesis of tert-butyl 2-[2-[2-[2-[[2-[2-(2,6-dioxo-3-piperidyl)-1,3-dioxo-isoindolin-4-yl]oxyacetyl]amino]ethoxy]ethoxy]ethoxy]acetate (Compound 2.01)

To a stirred solution of DIPEA (0.24 mL, 1.35 mmol) dissolved in THF (5 mL), under nitrogen atmosphere were added HOBt (91.42 mg, 0.6800 mmol), EDC·HCl (129.34 mg, 0.680 mmol), and 2-[2-(2,6-dioxo-3-piperidyl)-1,3-dioxo-isoindolin-4-yl]oxyacetic acid (Compound 2.03) (150 mg, 0.4500 mmol) at 0° C. and stirred for 10 min. At the same temperature, tert-butyl 2-[2-[2-(2-aminoethoxy)ethoxy]ethoxy]acetate (118.88 mg, 0.4500 mmol) was added, then the reaction mixture was allowed to stir at ambient temperature for 16 h. The progress of the reaction was monitored by TLC (5% MeOH in DCM, Product Rf=0.7, SM Rf=0.2) and LCMS. Reaction mixture was quenched with ice cold water, concentrated under reduced pressure, extracted with EtOAc (50 mL×2), and organic layer was washed with saturated brine solution (50 mL×1). The organic layer was dried over anhydrous Na₂SO₄ and concentrated. The crude residue was purified by Combiflash column chromatography (silica gel), eluent 5% MeOH in DCM to give tert-butyl 2-[2-[2-[2-[[2-[2-(2,6-dioxo-3-piperidyl)-1,3-dioxo-isoindolin-4-yl]oxyacetyl]amino]ethoxy]ethoxy]ethoxy]acetate (Compound 2.01) (110 mg, 42%) as a sticky solid. LCMS-ESI (pos.) m/z: 578.5 (M+1)⁺

### Synthesis of 2-[2-[2-[2-[[2-[2-(2,6-dioxo-3-piperidyl)-1,3-dioxo-isoindolin-4-yl]oxyacetyl]amino]ethoxy]ethoxy]ethoxy]acetic acid (Compound 2.02)

To a stirred solution of tert-butyl 2-[2-[2-[2-[[2-[2-(2,6-dioxo-3-piperidyl)-1,3-dioxo-isoindolin-4-yl]oxyacetyl]amino]ethoxy]ethoxy]ethoxy]acetate (110 mg, 0.1900 mmol) (Compound 2.01) in DCM (1 mL) at 0° C. was added TFA (0.73 mL, 9.52 mmol). The reaction was allowed to warm to ambient temperature and continuously stirred for 3 h. After completion of reaction, monitored by LCMS, TFA was removed under reduced pressure and the crude was triturated with n-pentane to give 2-[2-[2-[2-[[2-[2-(2,6-dioxo-3-piperidyl)-1,3-dioxo-isoindolin-4-yl]oxyacetyl]amino]ethoxy]ethoxy]ethoxy]acetic acid (Compound 2.02) (70 mg, 70%) as off-white solid. LCMS-ESI (pos.) m/z: 522.2 (M+1)⁺

### Synthesis of 2-[2-[2-[2-[[2-[2-(2,6-dioxo-3-piperidyl)-1,3-dioxo-isoindolin-4-yl]oxyacetyl]amino]ethoxy]ethoxy]ethoxy]—N-[(1S,3S)-3-[[5-(1-ethylpropyl)pyrazolo[1,5-a]pyrimidin-7-yl]amino]cyclopentyl]acetamide (Compound 2.0)

To a stirred solution of (1S,3S)—N3-[5-(1-ethylpropyl)pyrazolo[1,5-a]pyrimidin-7-yl]cyclopentane-1,3-diamine hydrochloride (33 mg, 0.1000 mmol) in DMF (0.27 mL), was added DIPEA (0.05 mL, 0.3800 mmol) and 2-[2-[2-[2-[[2-[2-(2,6-dioxo-3-piperidyl)-1,3-dioxo-isoindolin-4-yl]oxyacetyl]amino]ethoxy]ethoxy]ethoxy]acetic acid (Compound 2.02) (50 mg, 0.1000 mmol) at 0° C. HATU (43.75 mg, 0.1200 mmol) was then added. The reaction was stirred at ambient temperature for 16 h, then quenched with ice cold water, extracted with EtOAc (30 mL×1), and the organic layer was washed with saturated brine solution (30 mL×1). The organic layer was dried over anhydrous Na₂SO₄ and solvent concentrated and purified by Prep HPLC to give 2-[2-[2-[2-[[2-[2-(2,6-dioxo-3-piperidyl)-1,3-dioxo-isoindolin-4-yl]oxyacetyl]amino]ethoxy]ethoxy]ethoxy]-N-[(1S,3S)-3-[[5-(1-ethylpropyl)pyrazolo[1,5-a]pyrimidin-7-yl]amino]cyclopentyl]acetamide (Compound 2.0) (19 mg, 25%) as an off-white solid. LCMS-ESI (pos.) m/z: 791.9 (M+1)⁺

¹H NMR (400 MHz, DMSO-d₆) δ 10.78-10.75 (m, 1H), 7.96 (s, 1H), 7.80 (t, J=7.9 Hz, 1H), 7.73 (s, 1H), 7.50 (d, J=7.3 Hz, 1H), 7.45 (d, J=8.5 Hz, 1H), 7.41-7.34 (m, 1H), 7.15-7.09 (m, 1H), 6.30 (s, 1H), 5.94 (s, 1H), 5.07 (dd, J=5.5, 12.7 Hz, 1H), 4.76 (s, 2H), 4.38-4.19 (m, 1H), 3.87 (s, 2H), 3.62-3.51 (m, 10H), 3.37 (q, J=5.9 Hz, 2H), 2.92-2.82 (m, 1H), 2.70-2.55 (m, 3H), 2.46-2.36 (m, 1H), 2.35-2.18 (m, 1H), 2.12-2.00 (m, 4H), 1.77-1.61 (m, 6H), 0.82 (t, J=7.4 Hz, 6H).

### Compound 3.0 N-[(1S,3S)-3-[[3-chloro-5-(1-ethylpropyl)pyrazolo[1,5-a]pyrimidin-7-yl]amino]cyclopentyl]-2-[2-[2-[2-[[2-[2-(2,6-dioxo-3-piperidyl)-1,3-dioxo-isoindolin-4-yl]oxyacetyl]amino]ethoxy]ethoxy]ethoxy]acetamide

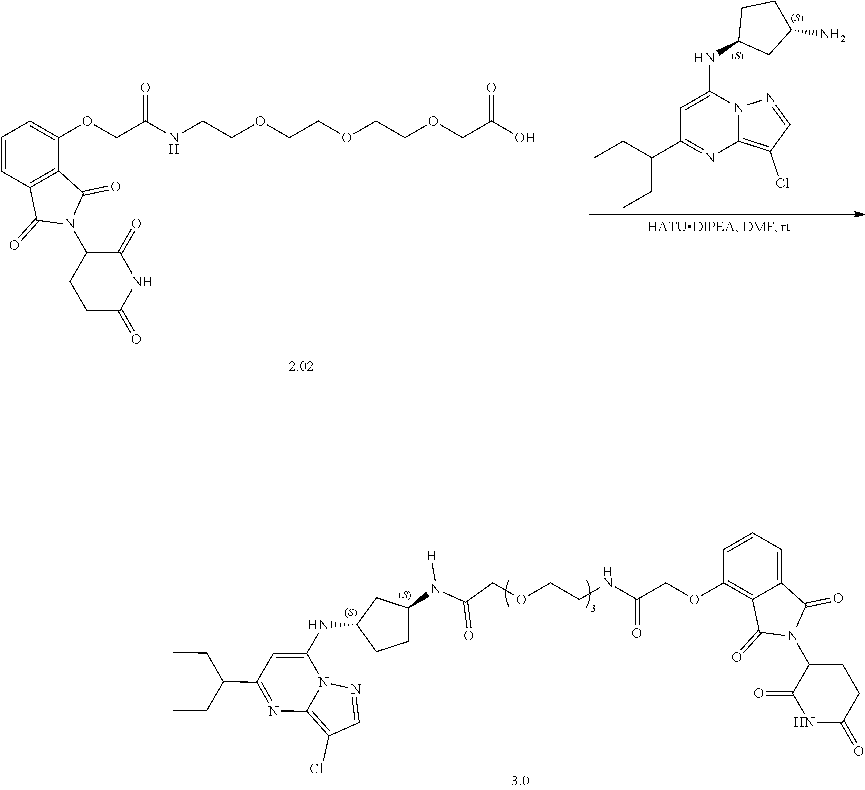

### Synthesis of N-[(1S,3S)-3-[[3-chloro-5-(1-ethylpropyl)pyrazolo[1,5-a]pyrimidin-7-yl]amino]cyclopentyl]-2-[2-[2-[2-[[2-[2-(2,6-dioxo-3-piperidyl)-1,3-dioxo-isoindolin-4-yl]oxyacetyl]amino]ethoxy]ethoxy]ethoxy]acetamide (Compound 3.0)

To a stirred solution of 2-[2-[2-[2-[[2-[2-(2,6-dioxo-3-piperidyl)-1,3-dioxo-isoindolin-4-yl]oxyacetyl]amino]ethoxy]ethoxy]ethoxy]acetic acid (Compound 2.02) (40 mg, 0.08 mmol) in DMF (0.22 mL), were added (1S,3S)—N3-[3-chloro-5-(1-ethylpropyl)pyrazolo[1,5-a]pyrimidin-7-yl]cyclopentane-1,3-diamine (25 mg, 0.07 mmol) (CAS #2416874-47-8, prepared as described in US20200131189 A1) and DIPEA (0.03 mL, 0.23 mmol) at 0° C. HATU (35 mg, 0.09 mmol) was then added, and the reaction mixture was stirred at ambient temperature for 16 h. The progress of the reaction was monitored by TLC (5% MeOH in DCM, Product R_{f}=0.7, SM R_{f}=0.4) and LCMS. Reaction mixture was quenched with ice-cold water and extracted with EtOAc (30 mL×2). The organic layer was washed with saturated brine solution (30 mL×1), dried over anhydrous Na₂SO₄, and evaporated in vacuo. The crude residue was purified by Prep HPLC using ammonium acetate buffer and MECN as mobile phase followed by lyophilization to give N-[(1S,3S)-3-[[3-chloro-5-(1-ethylpropyl)pyrazolo[1,5-a]pyrimidin-7-yl]amino]cyclopentyl]-2-[2-[2-[2-[[2-[2-(2,6-dioxo-3-piperidyl)-1,3-dioxo-isoindolin-4-yl]oxyacetyl]amino]ethoxy]ethoxy]ethoxy]acetamide (Compound 3.0) (10 mg, 15%). LCMS-ESI (pos.) m/z: 825.6 (M+1)⁺

¹H NMR (400 MHz, DMSO-d₆) δ 11.17-11.04 (m, 1H), 8.16 (s, 1H), 8.00 (s, 1H), 7.88 (d, J=7.6 Hz, 1H), 7.80 (t, J=7.9 Hz, 1H), 7.66 (d, J=8.0 Hz, 1H), 7.49 (d, J=7.3 Hz, 1H), 7.39 (d, J=8.7 Hz, 1H), 6.06 (s, 1H), 5.15-5.06 (m, 1H), 4.78 (s, 2H), 4.36-4.17 (m, 2H), 3.86 (s, 2H), 3.61-3.48 (m, 7H), 3.47 (t, J=5.8 Hz, 2H), 3.32-3.26 (m, 2H), 2.92-2.84 (m, 1H), 2.64-2.54 (m, 1H), 2.22-2.13 (m, 2H), 2.08-1.98 (m, 3H), 1.99-1.87 (m, 2H), 1.80-1.55 (m, 6H), 1.55-1.42 (m, 1H), 0.77 (t, J=7.3 Hz, 6H).

### Compound 4.0 N-[(1S,3S)-3-[[3-chloro-5-(1-ethylpropyl)pyrazolo[1,5-a]pyrimi-din-7-yl]amino]cyclopentyl]-2-[2-[2-[[2-[2-(2,6-dioxo-3-piperidyl)-1,3-dioxo-isoindolin-4-yl]oxyacetyl]amino]ethoxy]ethoxy]acetamide

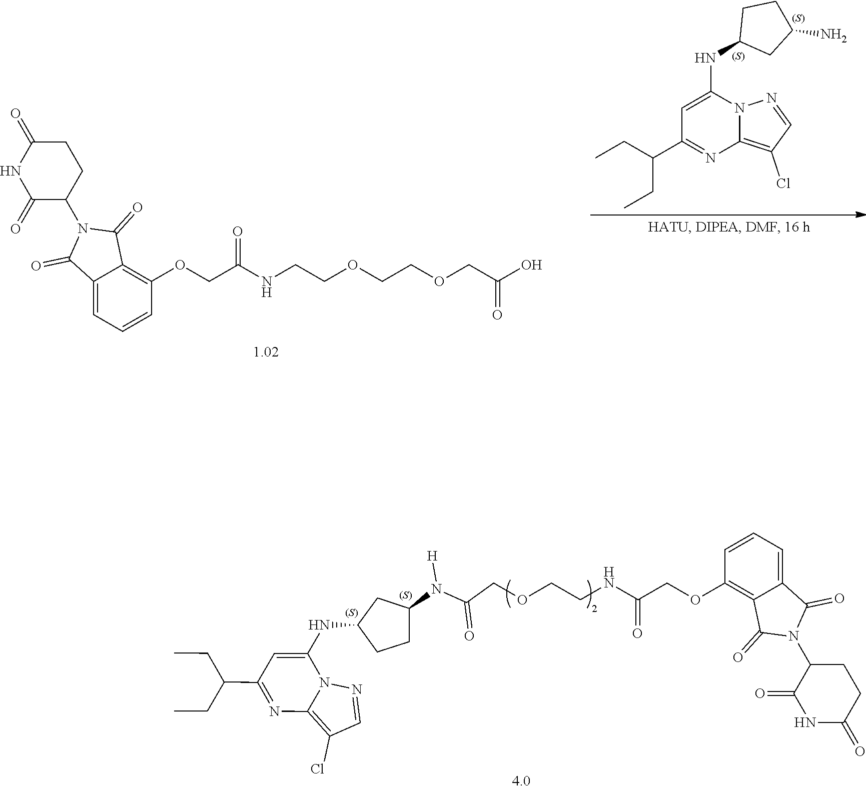

### Synthesis of N-[(1S,3S)-3-[[3-chloro-5-(1-ethylpropyl)pyrazolo[1,5-a]pyrimi-din-7-yl]amino]cyclopentyl]-2-[2-[2-[[2-[2-(2,6-dioxo-3-piperidyl)-1,3-dioxo-isoindolin-4-yl]oxyacetyl]amino]ethoxy]ethoxy]acetamide (Compound 4.0)

To a stirred solution of 2-[2-[2-[[2-[2-(2,6-dioxo-3-piperidyl)-1,3-dioxo-isoindolin-4-yl]oxyacetyl]amino]ethoxy]ethoxy]acetic acid (36.62 mg, 0.08 mmol) (Compound 1.02) in DMF (0.22 mL), were added (1S,3S)—N3-[3-chloro-5-(1-ethylpropyl)pyrazolo[1,5-a]pyrimidin-7-yl]cyclopentane-1,3-diamine (25 mg, 0.07 mmol) (CAS #2416874-47-8, prepared using the procedure as described in US20200131189 A1) and DIPEA (0.03 mL, 0.23 mmol) at 0° C. HATU (35 mg, 0.09 mmol) was then added. The reaction mixture was stirred at ambient temperature for 16 h, then quenched with ice-cold water and extracted with EtOAc (30 mL×2). The organic layer was washed with saturated brine solution (30 mL×1), dried over anhydrous Na₂SO₄, and evaporated in vacuo. The crude residue was purified by Prep-HPLC using ammonium acetate buffer and MeCN as mobile phase followed by lyophilization to give N-[(1S,3S)-3-[[3-chloro-5-(1-ethylpropyl)pyrazolo[1,5-a]pyrimi-din-7-yl]amino]cyclopentyl]-2-[2-[2-[[2-[2-(2,6-dioxo-3-piperidyl)-1,3-dioxo-isoindolin-4-yl]oxyacetyl]amino]ethoxy]ethoxy]acetamide (Compound 4.0) (17 mg, 27%). LCMS-ESI (pos.) m/z: 781.8 (M+1)⁺

¹H NMR (400 MHz, DMSO-d₆) δ 11.11 (s, 1H), 8.16 (s, 1H), 8.02 (t, J=5.6 Hz, 1H), 7.87 (d, J=7.6 Hz, 1H), 7.83-7.76 (m, 1H), 7.66 (d, J=7.9 Hz, 1H), 7.49 (d, J=7.2 Hz, 1H), 7.40 (d, J=8.6 Hz, 1H), 6.06 (d, J=3.2 Hz, 1H), 5.11 (dd, J=5.5, 12.9 Hz, 1H), 4.79 (s, 2H), 4.36-4.17 (m, 3H), 3.87 (s, 2H), 3.59 (s, 3H), 3.50 (t, J=5.7 Hz, 2H), 3.39-3.31 (m, 2H), 2.95-2.83 (m, 1H), 2.64-2.50 (m, 1H), 2.47-2.37 (m, 1H), 2.22-2.13 (m, 1H), 2.07-1.88 (m, 4H), 1.78-1.57 (m, 5H), 1.58-1.42 (m, 1H), 0.83-0.73 (m, 6H).

### Compound 5.0 N-[2-[2-[2-[2-[[2-(2,6-dioxo-3-piperidyl)-1,3-dioxo-isoindolin-4-yl]amino]ethoxy]ethoxy]ethoxy]ethyl]-2-[[(1S,3S)-3-[[5-(1-ethylpropyl)pyrazolo[1,5-a]pyrimidin-7-yl]amino]cyclopentyl]amino]acetamide

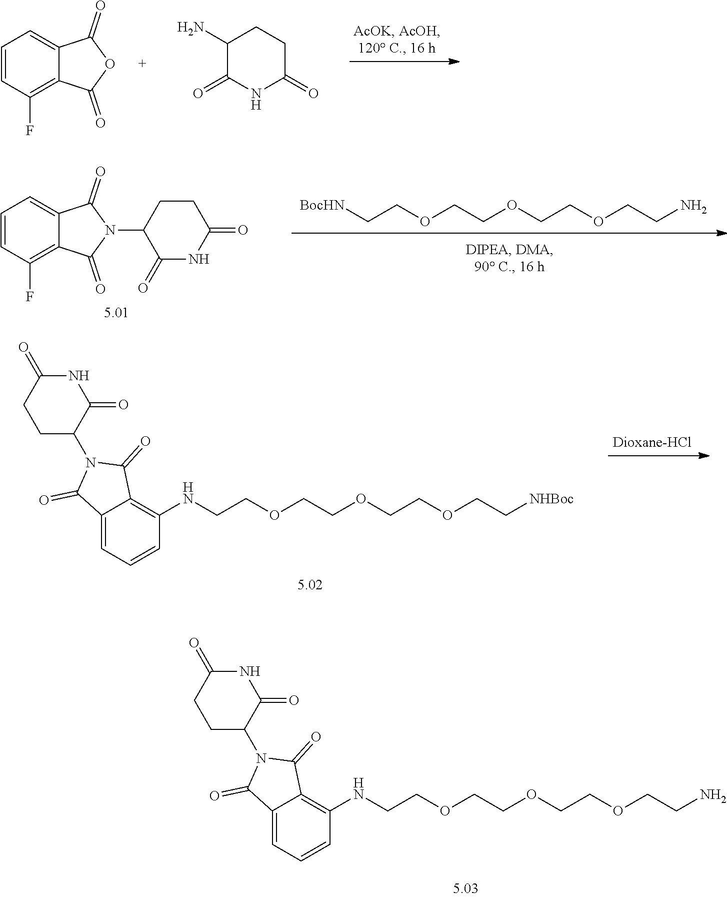 [SMILES: O=C1OC(=O)c2c(F)cccc21 ; NC1CCC(=O)NC1=O ; O=C1CCC(N2C(=O)c3cccc(F)c3C2=O)C(=O)N1 ; CC(C)(C)OC(=O)NCCOCCOCCOCCN ; CC(C)(C)OC(=O)NCCOCCOCCOCCNc1cccc2c1C(=O)N(C1CCC(=O)NC1=O)C2=O ; NCCOCCOCCOCCNc1cccc2c1C(=O)N(C1CCC(=O)NC1=O)C2=O]

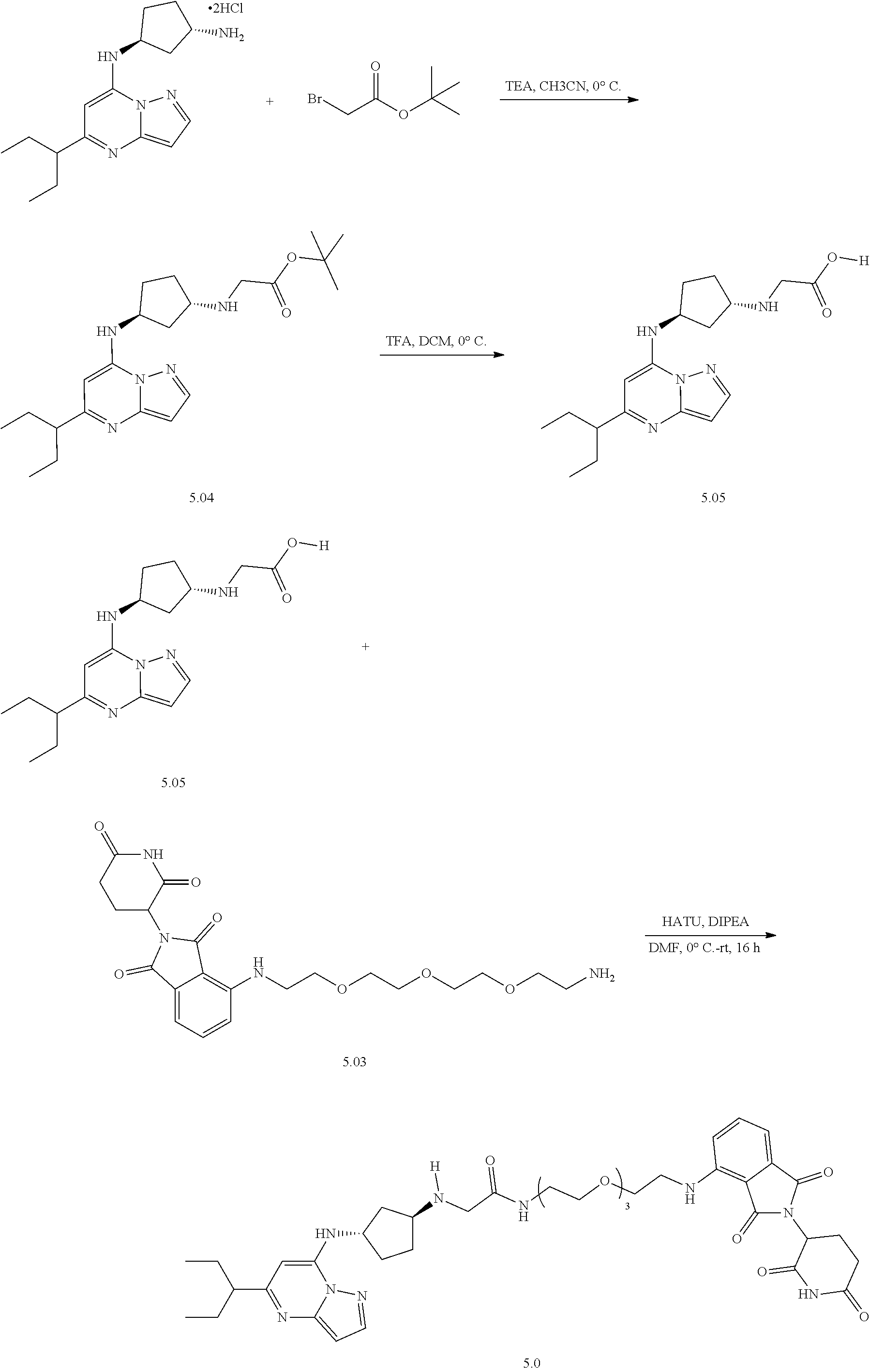

### Synthesis of 2-(2,6-dioxo-3-piperidyl)-4-fluoro-isoindoline-1,3-dione (Compound 5.01)

To a stirred solution of 4-fluoroisobenzofuran-1,3-dione (500 mg, 3.01 mmol) and 3-aminopiperidine-2,6-dione hydrochloride (495.42 mg, 3.01 mmol) in acetic acid (10 mL) was added potassium acetate (456.02 mg, 4.52 mmol) at room temperature. Then the reaction mixture was heated at 90° C. for 16 h. After completion of the reaction, the solvent was removed under reduced pressure and the crude residue was purified by Combiflash column chromatography (silica gel), eluent 5% MeOH in DCM to give 2-(2,6-dioxo-3-piperidyl)-4-fluoro-isoindoline-1,3-dione (Compound 5.01) (565 mg, 68%). LCMS-ESI (pos.) m/z: 277.0 (M+1)⁺

### Synthesis of tert-butyl N-[2-[2-[2-[2-[[2-(2,6-dioxo-3-piperidyl)-1,3-dioxo-isoindolin-4-yl]amino]ethoxy]ethoxy]ethoxy]ethyl]carbamate (Compound 5.02)

To a solution of 2-(2,6-dioxo-3-piperidyl)-4-fluoro-isoindoline-1,3-dione (500 mg, 1.81 mmol) (Compound 5.01) and tert-butyl N-[2-[2-[2-(2-aminoethoxy)ethoxy]ethoxy]ethyl]carbamate (0.6 mL, 2.17 mmol) [CAS: 101187-40-0] in DMA (10 mL) was added DIPEA (0.97 mL, 5.43 mmol). The reaction mixture was heated at 90° C. for 16 h. The reaction mixture was taken up in EtOAc, the organics washed with water (2×20 mL), followed by saturated brine solution (1×20 mL). The organic layer was separated and dried over anhydrous Na₂SO₄ and concentrated to dryness in vacuo. The crude product was purified by Combiflash column chromatography (silica gel), eluent 60% EtOAc in hexane. The desired fractions were concentrated to dryness in vacuo to give tert-butyl N-[2-[2-[2-[2-[[2-(2,6-dioxo-3-piperidyl)-1,3-dioxo-isoindolin-4-yl]amino]ethoxy]ethoxy]ethoxy]ethyl]carbamate (Compound 5.02) (600 mg, 60%). LCMS-ESI (pos.) m/z: 549.4 (M+1)⁺

### Synthesis of 4-[2-[2-[2-(2-aminoethoxy)ethoxy]ethoxy]ethylamino]-2-(2,6-dioxo-3-piperidyl)isoindoline-1,3-dione (Compound 5.03)

To a stirred solution of tert-butyl N-[2-[2-[2-[2-[[2-(2,6-dioxo-3-piperidyl)-1,3-dioxo-isoindolin-4-yl]amino]ethoxy]ethoxy]ethoxy]ethyl]carbamate (600 mg, 1.09 mmol) (Compound 5.02) in dioxane (3 mL) was added Dioxane-HCl (4M; 30 mL, 167.44 mmol) slowly at 0° C. and stirred at ambient temperature for 3 h. The reaction mixture was concentrated in vacuo to give 4-[2-[2-[2-(2-aminoethoxy)ethoxy]ethoxy]ethylamino]-2-(2,6-dioxo-3-piperidyl)isoindoline-1,3-dione (Compound 5.03) (480 mg, 98%). LCMS-ESI (pos.) m/z: 448.8 (M+1)⁺

### Synthesis of tert-butyl 2-[[(1S,3S)-3-[[5-(1-ethylpropyl)pyrazolo[1,5-a]pyrimidin-7-yl]amino]cyclopentyl]amino]acetate (Compound 5.04)

To a stirred solution of [(1S,3S)-3-[[5-(1-ethylpropyl)pyrazolo[1,5-a]pyrimidin-4-ium-7-yl]amino]cyclopentyl]ammonium dichloride (100 mg, 0.28 mmol) in MeCN (6.25 mL) were added K₂CO₃ (76.71 mg, 0.56 mmol), and tert-butyl 2-bromoacetate (64.96 mg, 0.3300 mmol) slowly at 0° C. and stirred at ambient temperature for 3 h. Reaction mixture was evaporated, and the residue diluted with MeCN and stirred at ambient temperature for 3 h. The reaction mixture was filtered through celite bed, washed with ethyl acetate, and the organic layer was concentrated to dryness in vacuo to give the crude tert-butyl 2-[[(1S,3S)-3-[[5-(1-ethylpropyl)pyrazolo[1,5-a]pyrimidin-7-yl]amino]cyclopentyl]amino]acetate (Compound 5.04) (80 mg, 72%). LCMS-ESI (pos.) m/z: 401.8 (M+1)⁺

### Synthesis of 2-[[(1S,3S)-3-[[5-(1-ethylpropyl)pyrazolo[1,5-a]pyrimidin-7-yl]amino]cyclopentyl]amino]acetic acid (Compound 5.05)

To a stirred solution of tert-butyl 2-[[(1S,3S)-3-[[5-(1-ethylpropyl)pyrazolo[1,5-a]pyrimidin-7-yl]amino]cyclopentyl]amino]acetate (60 mg, 0.15 mmol) (Compound 5.04) in DCM (2 mL) was added TFA (0.02 mL, 0.30 mmol) at 0° C. and stirred at ambient temperature for 3 h. The reaction mixture was concentrated under reduced pressure and triturated with n-pentane to give 2-[[(1S,3S)-3-[[5-(1-ethylpropyl)pyrazolo[1,5-a]pyrimidin-7-yl]amino]cyclopentyl]amino]acetic acid (50 mg, 97%) (Compound 5.05). LCMS-ESI (pos.) m/z: 346.3 (M+1)⁺

### Synthesis of N-[2-[2-[2-[2-[[2-(2,6-dioxo-3-piperidyl)-1,3-dioxo-isoindolin-4-yl]amino]ethoxy]ethoxy]ethoxy]ethyl]-2-[[(1S,3S)-3-[[5-(1-ethylpropyl)pyrazolo[1,5-a]pyrimidin-7-yl]amino]cyclopentyl]amino]acetamide (Compound 5.0)

To a stirred solution of 4-[2-[2-[2-(2-aminoethoxy)ethoxy]ethoxy]ethylamino]-2-(2,6-dioxo-3-piperidyl)isoindoline-1,3-dione (25 mg, 0.06 mmol) (Compound 5.03) and 2-[[(1S,3S)-3-[[5-(1-ethylpropyl)pyrazolo[1,5-a]pyrimidin-7-yl]amino]cyclopentyl]amino]acetic acid (23.11 mg, 0.07 mmol) (Compound 5.05) in DMF (1 mL) was added DIPEA (0.04 mL, 0.22 mmol) followed by HATU (42.39 mg, 0.11 mmol) at 0° C. and stirred at ambient temperature for 16 h. Then the reaction mixture was poured into ice-cold water and extracted with ethyl acetate. The organic layer was dried over anhydrous Na₂SO₄, concentrated under vacuum to give the crude, which was purified by Prep HPLC to give N-[2-[2-[2-[2-[[2-(2,6-dioxo-3-piperidyl)-1,3-dioxo-isoindolin-4-yl]amino]ethoxy]ethoxy]ethoxy]ethyl]-2-[[(1S,3S)-3-[[5-(1-ethylpropyl)pyrazolo[1,5-a]pyrimidin-7-yl]amino]cyclopentyl]amino]acetamide (Compound 5.0) (5 mg, 11%). LCMS-ESI (pos.) m/z: 776.9 (M+1)⁺

¹H NMR (400 MHz, DMSO-d₆) δ 11.24-10.88 (m, 2H), 7.98 (s, 1H), 7.85 (s, 1H), 7.62-7.53 (m, 1H), 7.46 (d, J=7.2 Hz, 1H), 7.13 (d, J=8.5 Hz, 1H), 7.03 (d, J=7.1 Hz, 1H), 6.59 (s, 1H), 6.30 (s, 1H), 5.98 (s, 1H), 5.11-4.99 (m, 1H), 4.23-4.16 (m, 1H), 3.64-3.36 (m, 12H), 2.26-2.11 (m, 4H), 2.08-1.95 (m, 2H), 1.92-1.82 (m, 8H), 1.74-1.55 (m, 6H), 1.47-1.28 (m, 2H), 0.76 (t, J=7.3 Hz, 6H).

### Compound 6.0 N-[2-[2-[2-[2-[[2-(2,6-dioxo-3-piperidyl)-1,3-dioxo-isoindolin-4-yl]amino]ethoxy]ethoxy]ethoxy]ethyl]-2-[[(1S,3S)-3-[[3-chloro-5-(1-ethylpropyl)pyrazolo[1,5-a]pyrimidin-7-yl]amino]cyclopentyl]amino]acetamide

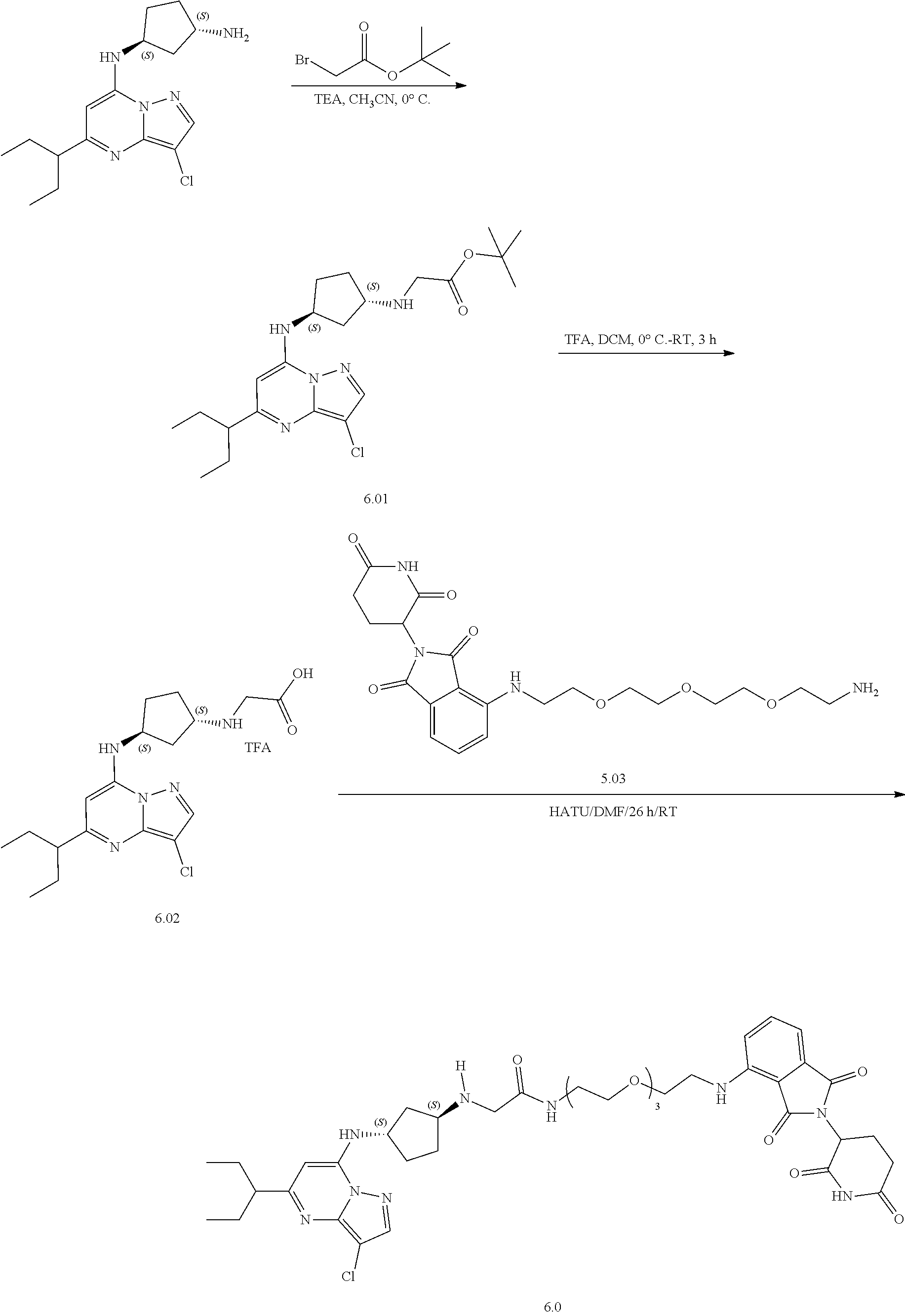

### Synthesis of tert-butyl 2-[[(1S,3S)-3-[[3-chloro-5-(1-ethylpropyl)pyrazolo[1,5-a]pyrimidin-7-yl]amino]cyclopentyl]amino]acetate (Compound 6.01)

To a stirred solution of (1S,3S)—N3-[3-chloro-5-(1-ethylpropyl)pyrazolo[1,5-a]pyrimidin-7-yl]cyclopentane-1,3-diamine hydrochloride (200 mg, 0.56 mmol) in MeCN (5 mL) under nitrogen atmosphere at 0° C. was added K₂CO₃ (154.28 mg, 1.12 mmol) followed by tert-butyl 2-bromoacetate (130.65 mg, 0.67 mmol) slowly at 0° C. and stirred for 3 h at ambient temperature. Reaction mixture was diluted with water (10 mL) and compound was extracted with EtOAc (30 mL). The solvent was removed under reduced pressure and the crude residue was purified by Combiflash column chromatography (silica gel), eluting with 10-15% EtOAc in hexane) to give tert-butyl 2-[[(1S,3S)-3-[[3-chloro-5-(1-ethylpropyl)pyrazolo[1,5-a]pyrimidin-7-yl]amino]cyclopentyl]amino]acetate (Compound 6.01) (120 mg, 44%).

LCMS-ESI (pos.) m/z: 437.2 (M+2)⁺

### Synthesis of 2-[[(1S,3S)-3-[[3-chloro-5-(1-ethylpropyl)pyrazolo[1,5-a]pyrimidin-7-yl]amino]cyclopentyl]amino]acetic acid 2,2,2-trifluoroacetic acid (Compound 6.02)

To a stirred solution of tert-butyl 2-[[(1S,3S)-3-[[3-chloro-5-(1-ethylpropyl)pyrazolo[1,5-a]pyrimidin-7-yl]amino]cyclopentyl]amino]acetate (120 mg, 0.28 mmol) (Compound 6.01) in DCM (2 mL) under nitrogen atmosphere, TFA (0.50 mL, 0.06 mmol) was added at 0° C. the reaction mixture was stirred at ambient temperature for 3 h. After completion of reaction, solvent and excess TFA was removed in vacuo. The residue was triturated with n-pentane (2×1 mL) and diethyl ether (2×1 mL) to give 2-[[(1S,3S)-3-[[3-chloro-5-(1-ethylpropyl)pyrazolo[1,5-a]pyrimidin-7-yl]amino]cyclopentyl]amino]acetic acid 2,2,2-trifluoroacetic acid (Compound 6.02) (95 mg, 85%). LCMS-ESI (pos.) m/z: 380.5 (M+1)⁺

### Synthesis of N-[2-[2-[2-[2-[[2-(2,6-dioxo-3-piperidyl)-1,3-dioxo-isoindolin-4-yl]amino]ethoxy]ethoxy]ethoxy]ethyl]-2-[[(1S,3S)-3-[[3-chloro-5-(1-ethylpropyl)pyrazolo[1,5-a]pyrimidin-7-yl]amino]cyclopentyl]amino]acetamide (Compound 6.0)

To the stirred solution of 2-[[(1S,3S)-3-[[3-chloro-5-(1-ethylpropyl)pyrazolo[1,5-a]pyrimidin-7-yl]amino]cyclopentyl]amino]acetic acid; 2,2,2-trifluoroacetic acid (Compound 6.02) (20 mg, 0.05 mmol) in DMF (1 mL) under nitrogen atmosphere were added 4-[2-[2-[2-(2-aminoethoxy)ethoxy]ethoxy]ethylamino]-2-(2,6-dioxo-3-piperidyl)isoindoline-1,3-dione (25.97 mg, 0.06 mmol) (Compound 5.03) followed by DIPEA (0.03 mL, 0.16 mol) at 0° C., and the reaction mixture was stirred for 5 min at 0° C. PyBop (32.88 mg, 0.06 mmol) was then added and the reaction mixture was stirred for 16 h at ambient temperature. The solvent was evaporated under reduced pressure, and the crude residue was purified by prep-HPLC using ammonium bicarbonate buffer and CH₃CN as mobile phase, followed by lyophilization to give N-[2-[2-[2-[2-[[2-(2,6-dioxo-3-piperidyl)-1,3-dioxo-isoindolin-4-yl]amino]ethoxy]ethoxy]ethoxy]ethyl]-2-[[(1S,3S)-3-[[3-chloro-5-(1-ethylpropyl)pyrazolo[1,5-a]pyrimidin-7-yl]amino]cyclopentyl]amino]acetamide (Compound 6.0) (10.5 mg, 23%). LCMS-ESI (pos.) m/z: 810.3 (M+1)⁺

¹H NMR (400 MHz, DMSO-d₆, 100° C.) δ 8.04 (s, 1H), 7.61-7.51 (m, 2H), 7.19-7.08 (m, 2H), 7.03 (d, J=7.1 Hz, 1H), 6.51 (s, 1H), 6.04 (s, 1H), 5.04-4.95 (m, 1H), 4.24 (q, J=7.2 Hz, 1H), 3.66 (t, J=5.5 Hz, 2H), 3.63-3.51 (m, 8H), 3.47 (q, J=4.8, 5.4 Hz, 4H), 3.33-3.22 (m, 3H), 3.13 (s, 2H), 2.90-2.80 (m, 1H), 2.68-2.51 (m, 3H), 2.32-2.18 (m, 2H), 2.13-1.94 (m, 2H), 1.96-1.89 (m, 2H), 1.81-1.61 (m, 6H), 1.52-1.38 (m, 1H), 0.84 (t, J=7.4 Hz, 6H).

### Compound 7.0 2-(2,6-dioxo-3-piperidyl)-4-[2-[2-[2-[2-[[(1S,3S)-3-[[5-(1-ethylpropyl)pyrazolo[1,5-a]pyrimidin-7-yl]amino]cyclopentyl]amino]ethoxy]ethoxy]ethoxy]ethylamino]isoindoline-1,3-dione

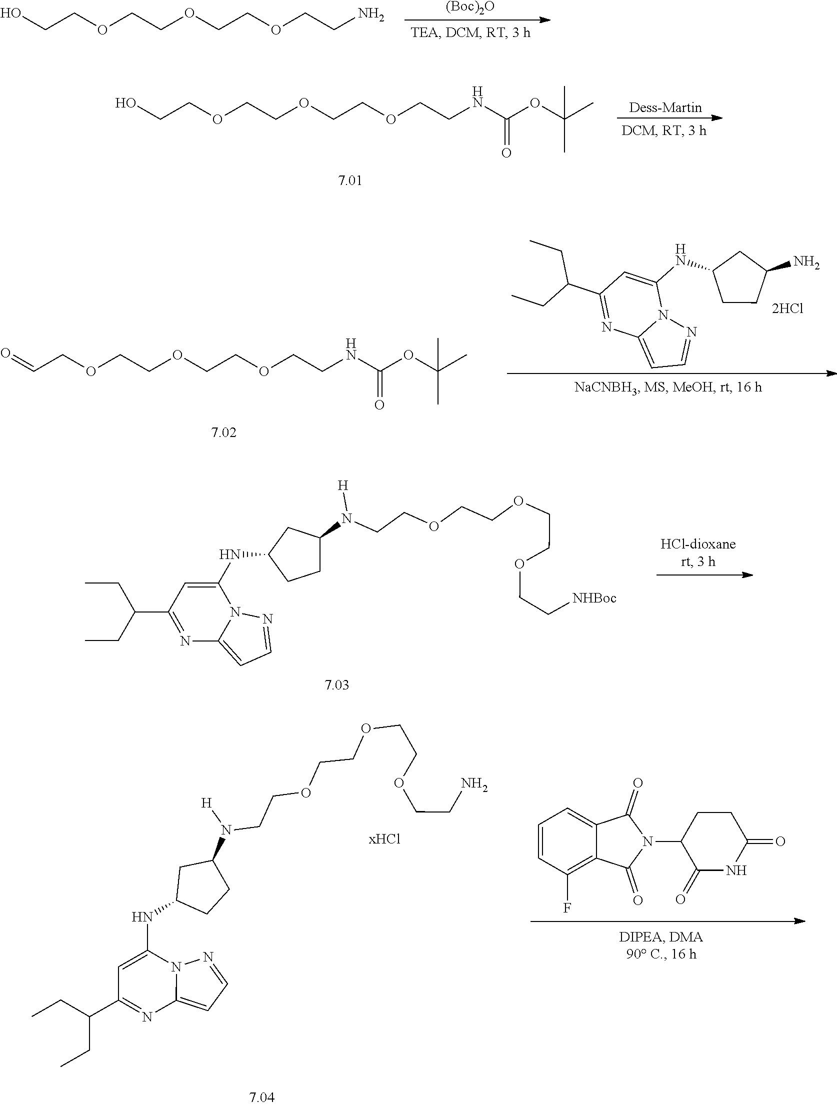

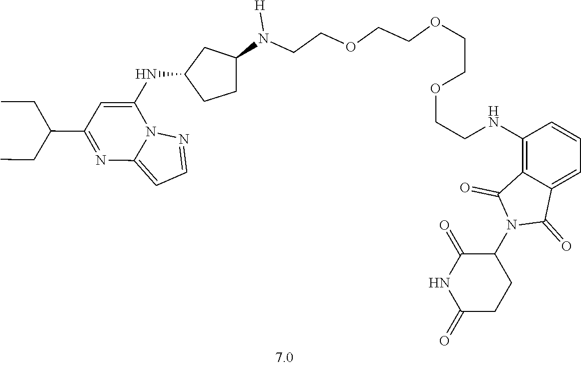 [SMILES: CCC(CC)c1cc(N[C@H]2CC[C@H](NCCOCCOCCOCCNc3cccc4c3C(=O)N(C3CCC(=O)NC3=O)C4=O)C2)n2nccc2n1]

### Step-1: Synthesis of tert-butyl N-[2-[2-[2-(2-hydroxyethoxy)ethoxy]ethoxy]ethyl]carbamate (Compound 7.01)

To a stirred solution of 2-[2-[2-(2-aminoethoxy)ethoxy]ethoxy]ethanol (250 mg, 1.29 mmol) in DCM (5 mL) were added Et₃N (0.36 mL, 2.59 mmol) and Boc-anhydride (0.36 mL, 1.55 mmol) slowly at 0° C. and stirred at ambient temperature for 3 h. The reaction mixture was concentrated to dryness, and the residue was taken up in DCM (30 mL) and washed with water (2×15 mL) followed by saturated brine solution (1×15 mL). The organic layer was separated, dried over anhydrous Na₂SO₄, and concentrated to dryness in vacuo. The crude was purified by Combiflash column chromatography (silica gel), eluent 5% MeOH in DCM. The desired fractions were concentrated to dryness in vacuo to give tert-butyl N-[2-[2-[2-(2-hydroxyethoxy)ethoxy]ethoxy]ethyl]carbamate (Compound 7.01) (230 mg, 61%). L CMS-ESI (pos.) m/z: 294.4 (M+1)⁺

### Step-2: Synthesis of tert-butyl N-[2-[12-[2-(2-oxoethoxy)ethoxy]ethoxy]ethyl]carbamate (Compound 7.02)

To a stirred solution of tert-butyl N-[2-[2-[2-(2-hydroxyethoxy)ethoxy]ethoxy]ethyl]carbamate (Compound 7.01) (280 mg, 0.95 mmol) in DCM (8 mL) was added Dess-Martin periodinane (485.8 mg, 1.15 mmol) at 0° C. and stirred at ambient temperature for 3 h. The reaction was concentrated to dryness in vacuo and the residue was taken up in DCM (25 mL) and washed with saturated NaHCO₃ solution (2×25 mL) followed by saturated brine solution (1×25 mL). The organic layer was separated and dried over anhydrous Na₂SO₄ and concentrated to dryness in vacuo. The crude was purified by Combiflash column chromatography (silica gel), eluent 5% MeOH in DCM. The desired fractions were concentrated to dryness in vacuo to give tert-butyl N-[2-[2-[2-(2-oxoethoxy)ethoxy]ethoxy]ethyl]carbamate (Compound 7.02). (187 mg, 67%). LCMS-ESI (pos.) m/z: 292.2 (M+1)⁺

### Step-3: Synthesis of tert-butyl N-[2-[2-[2-[2-[[(1S,3S)-3-[[5-(1-ethylpropyl)pyrazolo[1,5-a]pyrimidin-7-yl]amino]cyclopentyl]amino]ethoxy]ethoxy]ethoxy]ethyl]carbamate (Compound 7.03)

To a stirred solution of [(1S,3S)-3-[[5-(1-ethylpropyl)pyrazolo[1,5-a]pyrimidin-4-ium-7-yl]amino]cyclopentyl]ammonium dichloride (267.15 mg, 0.74 mmol) (CAS #2416873-83-9, prepared using the procedure as described in US20200131189 A1), 4 ÅMS in methanol (5 mL) at 0° C. was added sodium cyanoborohydride (116.47 mg, 1.85 mmol). The reaction mixture was allowed to warm to room temperature and continuously stirred at room temperature for 0.5 h, then added tert-butyl N-[2-[2-[2-(2-oxoethoxy)ethoxy]ethoxy]ethyl]carbamate (180 mg, 0.62 mmol) and stirred at ambient temperature for 16 h. The reaction mixture was filtered, and the filtrate was concentrated to dryness in vacuo. The crude was purified by Combiflash column chromatography (silica gel), eluent 5% MeOH in DCM. The desired fractions were concentrated to dryness in vacuo to give tert-butyl N-[2-[2-[2-[2-[[(1S,3S)-3-[[5-(1-ethylpropyl)pyrazolo[1,5-a]pyrimidin-7-yl]amino]cyclopentyl]amino]ethoxy]ethoxy]ethoxy]ethyl]carbamate (Compound 7.03) (67 mg, 15%). LCMS-ESI (pos.) m/z: 563.2 (M+1)⁺

### Step-4: Synthesis of 2-[2-[2-[2-[[(1S,3S)-3-[[5-(1-ethylpropyl)pyrazolo[1,5-a]pyrimidin-7-yl]amino]cyclopentyl]amino]ethoxy]ethoxy]ethoxy]ethylammonium chloride (Compound 7.04)

To a stirred solution of tert-butyl N-[2-[2-[2-[2-[[(1S,3S)-3-[[5-(1-ethylpropyl)pyrazolo[1,5-a]pyrimidin-7-yl]amino]cyclopentyl]amino]ethoxy]ethoxy]ethoxy]ethyl]carbamate (60 mg, 0.11 mmol) (Compound 7.03) in dioxane (0.5 mL) at 0° C. was added dioxane-HCl (4M; 0.08 mL, 0.4300 mmol). The reaction was allowed to warm to room temperature and continuously stirred for 3 h. The reaction mixture was concentrated in vacuo, washed with pentane, and dried in vacuo followed by lyophilization to give 2-[2-[2-[2-[[(1S,3S)-3-[[5-(1-ethylpropyl)pyrazolo[1,5-a]pyrimidin-7-yl]amino]cyclopentyl]amino]ethoxy]ethoxy]ethoxy]ethylammonium chloride (Compound 7.04) (20 mg, 38%). LCMS-ESI (neg.) m/z: 461.4 (M−1)⁺

### Step-5: Synthesis of 2-(2,6-dioxo-3-piperidyl)-4-[2-[2-[2-[2-[[(1S,3S)-3-[[5-(1-ethylpropyl)pyrazolo[1,5-a]pyrimidin-7-yl]amino]cyclopentyl]amino]ethoxy]ethoxy]ethoxy]ethylamino]isoindoline-1,3-dione (Compound 7.0)

To a stirred solution of 2-[2-[2-[2-[[(1S,3S)-3-[[5-(1-ethylpropyl)pyrazolo[1,5-a]pyrimidin-7-yl]amino]cyclopentyl]amino]ethoxy]ethoxy]ethoxy]ethylammonium chloride (20 mg, 0.04 mmol) (Compound 7.04) in DMA (2 mL) at 0° C. was added DIPEA (0.02 mL, 0.12 mmol). The reaction was allowed to warm to room temperature and added 2-(2,6-dioxo-3-piperidyl)-4-fluoro-isoindoline-1,3-dione (Compound 5.01) (22.14 mg, 0.08 mmol). The reaction mixture was then stirred at 90° C. for 16 h, whereupon LC-MS revealed reaction was incomplete. Further 2-(2,6-dioxo-3-piperidyl)-4-fluoro-isoindoline-1,3-dione (22.14 mg, 0.08 mmol) (Compound 5.01) was added and stirred at 90° C. for 16 h, whereupon LC-MS revealed reaction was complete. The reaction mixture was purified by Prep-HPLC followed by lyophilization MeCN:H₂O to give 2-(2,6-dioxo-3-piperidyl)-4-[2-[2-[2-[2-[[(1S,3S)-3-[[5-(1-ethylpropyl)pyrazolo[1,5-a]pyrimidin-7-yl]amino]cyclopentyl]amino]ethoxy]ethoxy]ethoxy]ethylamino]isoindoline-1,3-dione (Compound 7.0) (1.6 mg, 5%). LCMS-ESI (pos.) m/z: 719.4 (M+1)⁺

¹H NMR (400 MHz, DMSO-d₆) δ 7.99 (d, J=2.4 Hz, 1H), 7.57 (t, J=7.9 Hz, 1H), 7.45 (d, J=7.5 Hz, 1H), 7.14 (d, J=8.5 Hz, 1H), 7.03 (d, J=7.1 Hz, 1H), 6.61 (s, 1H), 6.31 (d, J=2.4 Hz, 1H), 5.97 (s, 1H), 5.12-5.00 (m, 1H), 4.22-4.07 (m, 1H), 3.66-3.40 (m, 10H), 3.24-3.14 (m, 2H), 3.07-2.77 (m, 4H), 2.70-2.58 (m, 4H), 2.45-2.35 (m, 2H), 2.21-2.09 (m, 2H), 2.07-1.78 (m, 5H), 1.74-1.54 (m, 4H), 1.43-1.28 (m, 1H), 0.76 (t, J=7.3 Hz, 6H).

### Compound 8.0 2-(2,6-dioxo-3-piperidyl)-4-[2-[2-[2-[2-[2-[[(1S,3S)-3-[[5-(1-ethylpropyl)pyrazolo[1,5-a]pyrimidin-7-yl]amino]cyclopentyl]amino]ethoxy]ethoxy]ethoxy]ethoxy]ethylamino]isoindoline-1,3-dione

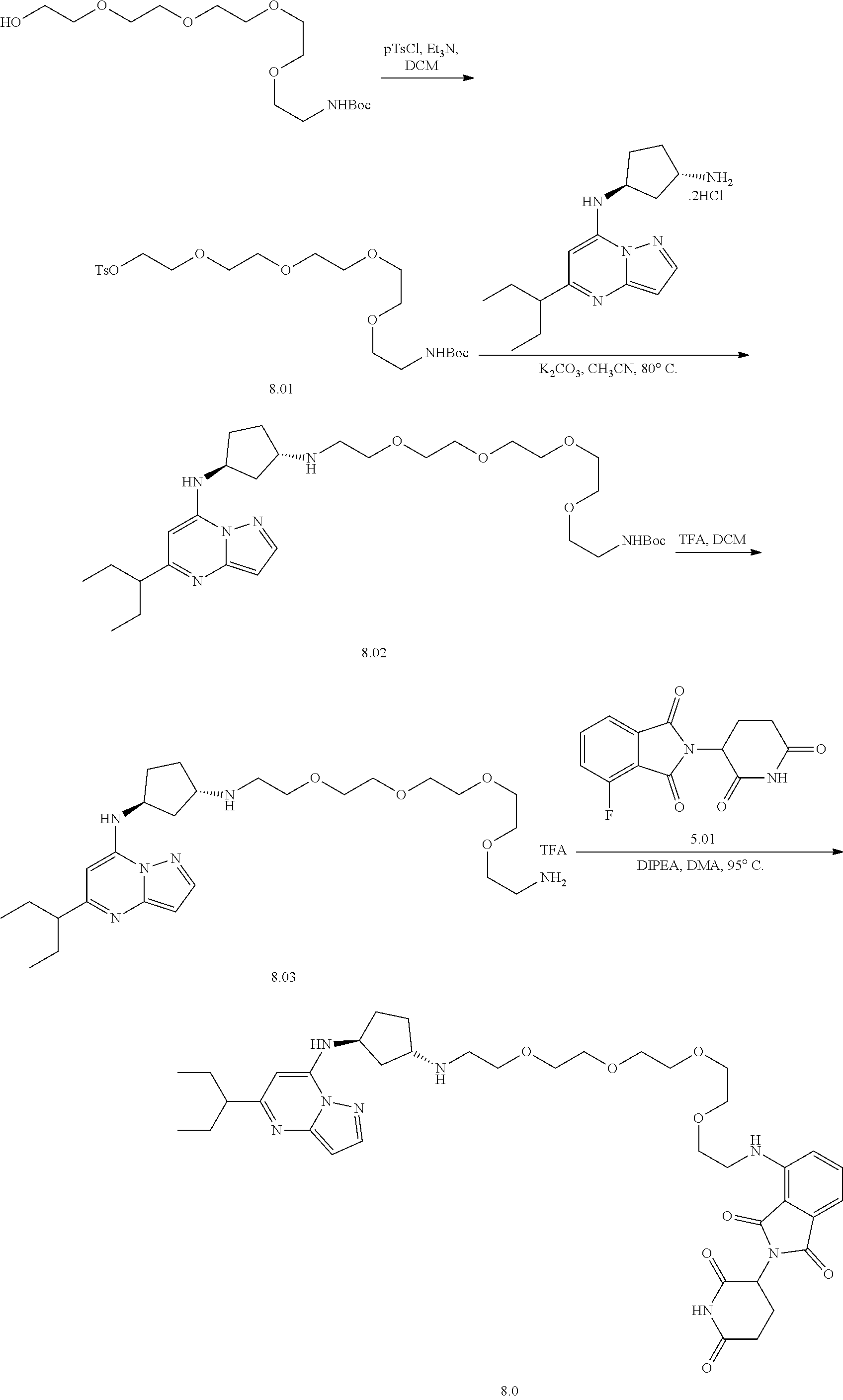

### Step-1: Synthesis of 2-[2-[2-[2-[2-(tert-butoxycarbonylamino)ethoxy]ethoxy]ethoxy]ethoxy]ethyl 4-methylbenzenesulfonate (Compound 8.01)

To a stirred solution of tert-butyl N-[2-[2-[2-[2-(2-hydroxyethoxy)ethoxy]ethoxy]ethoxy]ethyl]carbamate (280 mg, 0.83 mmol)[CAS: 1404111-67-6] in DCM (25 mL) at 0° C., were added p-toluenesulfonyl chloride (316.42 mg, 1.66 mmol) and Et₃N (0.47 mL, 3.32 mmol), and the reaction mixture was stirred at 0° C. for another 30 min. Then the reaction mixture was stirred at ambient temperature for 16 h. After completion of the reaction, water (20 mL) was added to the reaction mixture and extracted with DCM (30 mL). The organic layer was washed with brine, dried over anhydrous Na₂SO₄, and concentrated in vacuo. The crude product was purified by Combiflash column chromatography (silica gel), eluent 60% EtOAc in hexane, to give 2-[2-[2-[2-[2-(tert-butoxycarbonylamino)ethoxy]ethoxy]ethoxy]ethoxy]ethyl 4-methylbenzenesulfonate (Compound 8.01) (250 mg, 61%). LCMS-ESI (pos.) m/z: 492.0 (M+1)⁺

### Step-2: Synthesis of tert-butyl N-[2-[2-[2-[2-[2-[[(1S,3S)-3-[[5-(1-ethylpropyl)pyrazolo[1,5-a]pyrimidin-7-yl]amino]cyclopentyl]amino]ethoxy]ethoxy]ethoxy]ethoxy]ethyl]carbamate (Compound 8.02

To a stirred solution of (1S,3S)—N3-[5-(1-ethylpropyl)pyrazolo[1,5-a]pyrimidin-7-yl]cyclopentane-1,3-diamine dihydrochloride (180 mg, 0.50 mmol) (CAS #2416873-83-9, prepared using the procedure as described in US20200131189 A1) and 2-[2-[2-[2-[2-(tert-butoxycarbonylamino)ethoxy]ethoxy]ethoxy]ethoxy]ethyl 4-methylbenzenesulfonate (245.57 mg, 0.50 mmol) (Compound 8.01) in MeCN (10 mL), was added potassium carbonate (689.37 mg, 5.00 mmol) and the reaction mixture was heated at 85° C. for 36 h. The reaction mixture was then concentrated in vacuo and the crude was purified by Combiflash column chromatography (silica gel), eluent 10% MeOH in DCM to give tert-butyl N-[2-[2-[2-[2-[2-[[(1S,3S)-3-[[5-(1-ethylpropyl)pyrazolo[1,5-a]pyrimidin-7-yl]amino]cyclopentyl]amino]ethoxy]ethoxy]ethoxy]ethoxy]ethyl]carbamate (Compound 8.02) (165 mg, 54%). LCMS-ESI (pos.) m/z: 607.0 (M+1)⁺

### Step-3: Synthesis of (1S,3S)—N1-[2-[2-[2-[2-(2-aminoethoxy)ethoxy]ethoxy]ethoxy]ethyl]—N3-[5-(1-ethylpropyl)pyrazolo[1,5-a]pyrimidin-7-yl]cyclopentane-1,3-diamine (Compound 8.03)

To a stirred solution of tert-butyl N-[2-[2-[2-[2-[2-[[(1S,3S)-3-[[5-(1-ethylpropyl)pyrazolo[1,5-a]pyrimidin-7-yl]amino]cyclopentyl]amino]ethoxy]ethoxy]ethoxy]ethoxy]ethyl]carbamate (165 mg, 0.27 mmol) in DCM (3 mL) at 0° C., was added trifluoroacetic acid (0.55 mL, 7.19 mmol) and the reaction mixture was stirred at 0° C. for another 30 min. The reaction was then stirred at room temperature for 3 h. After completion of reaction, solvent was evaporated in vacuo to give (1S,3S)—N1-[2-[2-[2-[2-(2-aminoethoxy)ethoxy]ethoxy]ethoxy]ethyl]—N3-[5-(1-ethylpropyl)pyrazolo[1,5-a]pyrimidin-7-yl]cyclopentane-1,3-diamine (Compound 8.03) (125 mg, 91% crude). The compound used in the next step without further purification. LCMS-ESI (pos.) m/z: 507.3 (M+1)⁺

### Step-4: Synthesis of 2-(2,6-dioxo-3-piperidyl)-4-[2-[2-[2-[2-[2-[[(1S,3S)-3-[[5-(1-ethylpropyl)pyrazolo[1,5-a]pyrimidin-7-yl]amino]cyclopentyl]amino]ethoxy]ethoxy]ethoxy]ethoxy]ethylamino]isoindoline-1,3-dione (Compound 8.0)

A stirred solution of 2-(2,6-dioxo-3-piperidyl)-4-fluoro-isoindoline-1,3-dione (26.17 mg, 0.09 mmol) in DMA (1 mL) in a sealed tube were added (1S,3S)—N1-[2-[2-[2-[2-(2-aminoethoxy)ethoxy]ethoxy]ethoxy]ethyl]—N3-[5-(1-ethylpropyl)pyrazolo[1,5-a]pyrimidin-7-yl]cyclopentane-1,3-diamine (40 mg, 0.08 mmol) (Compound 8.03) and DIPEA (0.07 mL, 0.39 mmol) in DMA (2 mL). The reaction mixture was then heated at 95° C. for 16 h. After completion of reaction as revealed by LCMS, the reaction mixture was cooled, poured into ice-cold water and stirred for few minutes. The reaction mixture was extracted with EtOAc and washed with brine. The organic layer was dried over anhydrous Na₂SO₄ and concentrated in vacuo to give the crude. The crude product was purified by Prep-HPLC using ammonium acetate buffer/MECN mobile phase, followed by lyophilization to give 2-(2,6-dioxo-3-piperidyl)-4-[2-[2-[2-[2-[2-[[(1S,3S)-3-[[5-(1-ethylpropyl)pyrazolo[1,5-a]pyrimidin-7-yl]amino]cyclopentyl]amino]ethoxy]ethoxy]ethoxy]ethoxy]ethylamino]isoindoline-1,3-dione (Compound 8.0) (8 mg, 13%). LCMS-ESI (pos.) m/z: 763.3 (M+1)³⁰

¹H NMR (400 MHz, DMSO-d₆, 100° C.) 6 7.95 (d, J=1.8 Hz, 1H), 7.57 (t, J=7.9 Hz, 1H), 7.12 (d, J=8.6 Hz, 1H), 7.04 (d, J=7.0 Hz, 1H), 6.89 (d, J=7.0 Hz, 1H), 6.57-6.46 (m, 1H), 6.29 (d, J=2.1 Hz, 1H), 5.94 (s, 1H), 5.00 (dd, J=5.5, 12.5 Hz, 1H), 4.20 (q, J=7.0 Hz, 1H), 3.67 (t, J=5.5 Hz, 2H), 3.63-3.50 (m, 10H), 3.48 (q, J=5.5 Hz, 4H), 3.29 (t, J=6.2 Hz, 1H), 2.90-2.77 (m, 2H), 2.69 (t, J=5.8 Hz, 2H), 2.67-2.56 (m, 2H), 2.28-2.17 (m, 1H), 2.11-1.90 (m, 2H), 1.94-1.85 (m, 4H), 1.79-1.60 (m, 5H), 1.50-1.36 (m, 1H), 0.83 (t, J=7.4 Hz, 6H).

### Compound 9.0 2-(2,6-dioxo-3-piperidyl)-4-[2-[2-[2-[4-[[[(1S,3S)-3-[[5-(1-ethylpropyl)pyrazolo[1,5-a]pyrimidin-7-yl]amino]cyclopentyl]amino]methyl]triazol-1-yl]ethoxy]ethoxy]ethylamino]isoindoline-1,3-dione

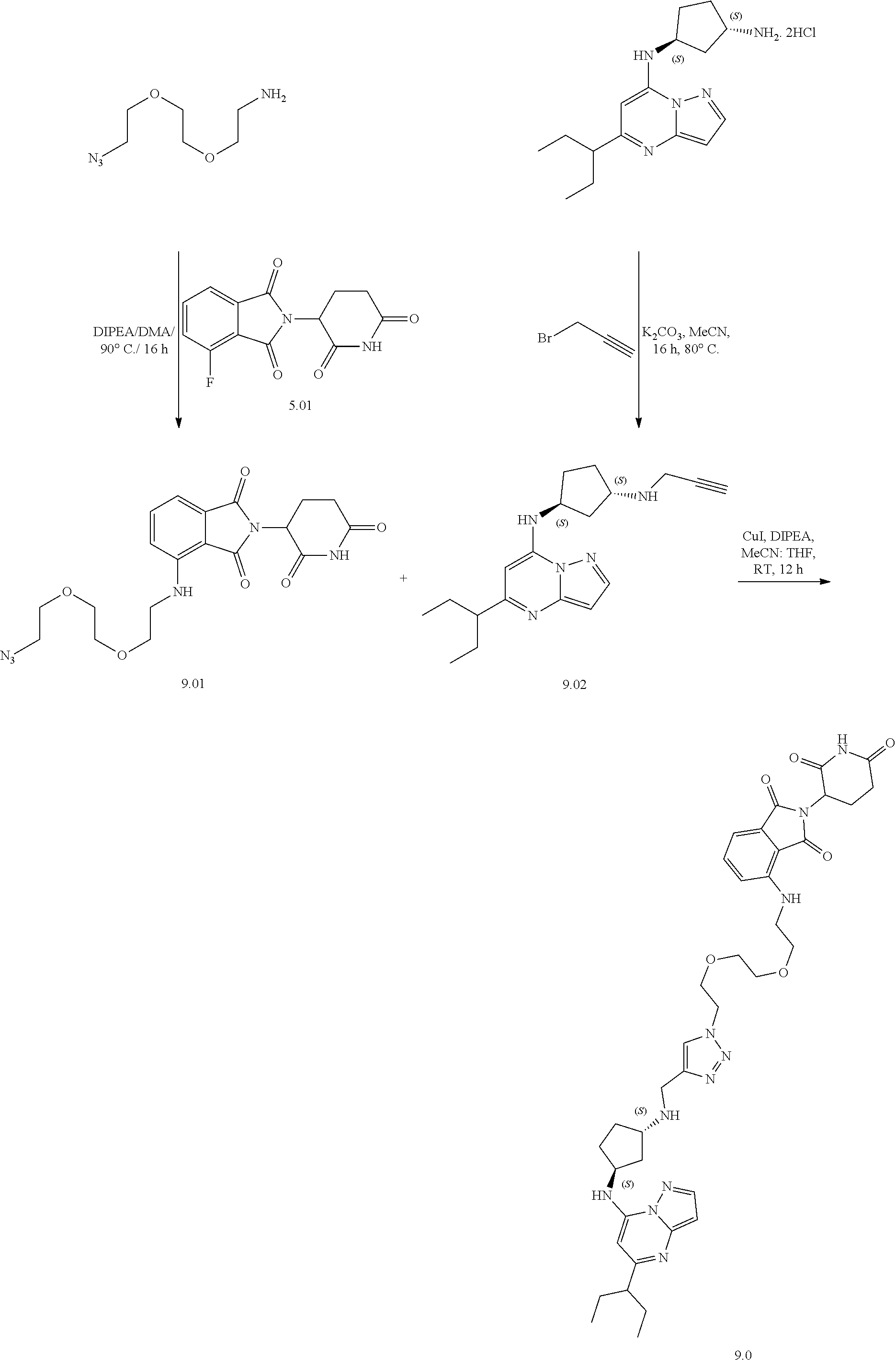

### Step-1: Synthesis of 4-[2-[12-[2-(2-azidoethoxy)ethoxy]ethoxy]ethylamino]-2-(2,6-dioxo-3-piperidyl)isoindoline-1,3-dione (Compound 9.01)

To a stirred solution of 2-[2-[2-(2-azidoethoxy)ethoxy]ethoxy]ethanamine (39.51 mg, 0.1800 mmol) in DMA (1 mL) and 2-(2,6-dioxo-3-piperidyl)-4-fluoro-isoindoline-1,3-dione (50 mg, 0.1800 mmol) (Compound 5.01), DIPEA (0.2 mL, 1.086 mmol) was added and the mixture was stirred for 16 h at 90° C. Reaction mixture was quenched with ice cold water, extracted with EtOAc (30 mL×2), and the organic layer was washed with saturated brine solution (50 mL×1). The organic layer was dried over anhydrous Na₂SO₄ and solvent concentrated and purified by Combiflash column chromatography (silica gel), eluent 70% EtOAc in hexane to give 4-[2-[2-[2-(2-azidoethoxy)ethoxy]ethoxy]ethylamino]-2-(2,6-dioxo-3-piperidyl)isoindoline-1,3-dione (Compound 9.01) (45 mg, 52%) as a bright yellow solid. LCMS-ESI (pos.) m/z: 431.2 (M+1)⁺

### Step-2: Synthesis of (1S,3S)—N3-[5-(1-ethylpropyl)pyrazolo[1,5-a]pyrimidin-7-yl]—N1-prop-2-ynyl-cyclopentane-1,3-diamine (Compound 9.02)

To a stirred solution of (1S,3S)—N3-[5-(1-ethylpropyl)pyrazolo[1,5-a]pyrimidin-7-yl]cyclopentane-1,3-diamine dihydrochloride (10 mg, 0.036 mmol) (CAS #2416873-83-9, prepared as described in US20200131189 A1) and Et₃N (0.01 mL, 0.0500 mmol) in DMF (1 mL) at 0° C. was added 3-bromoprop-1-yne (2.9 μL, 0.033 mmol). The reaction was heated to 70° C. and continuously stirred for 16 h, whereupon LC-MS revealed reaction was completed. The reaction mixture was concentrated to dryness, and to the residue was added ice cold water and extracted with EtOAc (1×20 mL). The organic layer was separated and washed with ice cold water (2×10 mL), followed by saturated brine solution (1×10 mL). The organic layer was dried over anhydrous Na₂SO₄ and concentrated to dryness. The residue was purified by Combiflash column chromatography (silica gel), eluent 70% EtOAc in Hexane to give to give (1S,3S)—N3-[5-(1-ethylpropyl)pyrazolo[1,5-a]pyrimidin-7-yl]—N1-prop-2-ynyl-cyclopentane-1,3-diamine (Compound 9.02) (10 mg, 18%) as a solid compound. LCMS-ESI (pos.) m/z: 326.2 (M+1)⁺

### Step-3: Synthesis of 2-(2,6-dioxo-3-piperidyl)-4-[2-[2-[2-[4-[[[(1S,3S)-3-[[5-(1-ethylpropyl)pyrazolo[1,5-a]pyrimidin-7-yl]amino]cyclopentyl]amino]methyl]triazol-1-yl]ethoxy]ethoxy]ethylamino]isoindoline-1,3-dione (Compound 9.0)

In an oven-dry 10 mL round-bottom flask, (1S,3S)—N3-[5-(1-ethylpropyl)pyrazolo[1,5-a]pyrimidin-7-yl]—N1-prop-2-ynyl-cyclopentane-1,3-diamine (35 mg, 0.1100 mmol) (Compound 9.02), 4-[2-[2-(2-azidoethoxy)ethoxy]ethylamino]-2-(2,6-dioxo-3-piperidyl)isoindoline-1,3-dione (46.29 mg, 0.1100 mmol) (Compound 9.01) and CuI (12.07 mg, 0.1100 mmol) were added to a mixture of MeCN (1 mL) and THF (1 mL). DIPEA (0.02 mL, 0.1100 mmol) was added, and the reaction mixture was vigorously stirred at ambient temperature for 12 h under an argon atmosphere. The reaction mixture was concentrated to dryness, and the residue was dissolved in EtOAc (20 mL) and washed with water (2×10 mL), followed by saturated brine solution (1×10 mL). The organic layer was separated, dried over anhydrous Na₂SO₄, and concentrated to dryness. The crude residue was purified by Prep-HPLC to give 2-(2,6-dioxo-3-piperidyl)-4-[2-[2-[2-[4-[[[(1S,3S)-3-[[5-(1-ethylpropyl)pyrazolo[1,5-a]pyrimidin-7-yl]amino]cyclopentyl]amino]methyl]triazol-1-yl]ethoxy]ethoxy]ethylamino]isoindoline-1,3-dione (Compound 9.0) (7 mg, 9%). LCMS-ESI (pos.) m/z: 756.4 (M+1)⁺

¹H NMR (400 MHz, DMSO-d₆) δ 7.99 (d, J=2.2 Hz, 1H), 7.89 (s, 1H), 7.61-7.52 (m, 1H), 7.43 (d, J=7.6 Hz, 1H), 7.12 (d, J=8.6 Hz, 1H), 7.03 (d, J=7.1 Hz, 1H), 6.59 (t, J=5.8 Hz, 1H), 6.30 (d, J=2.2 Hz, 1H), 5.97 (s, 1H), 5.04 (dd, J=5.4, 12.9 Hz, 1H), 4.47 (t, J=5.2 Hz, 2H), 4.17 (d, J=7.7 Hz, 1H), 3.80 (t, J=5.2 Hz, 2H), 3.76-3.65 (m, 2H), 3.58 (t, J=5.3 Hz, 2H), 3.53 (s, 3H), 3.46-3.42 (m, 2H), 2.94-2.80 (m, 2H), 2.62-2.52 (m, 2H), 2.48-2.36 (m, 2H), 2.17 (s, 1H), 2.06-1.78 (m, 5H), 1.73-1.54 (m, 5H), 1.49-1.34 (m, 1H), 0.76 (t, J=7.3 Hz, 6H).

### Compound 10.0 2-(2,6-dioxo-3-piperidyl)-4-[2-[2-[2-[2-[4-[[[(1S,3S)-3-[[5-(1-ethylpropyl)pyrazolo[1,5-a]pyrimidin-7-yl]amino]cyclopentyl]amino]methyl]triazol-1-yl]ethoxy]ethoxy]ethoxy]ethylamino]isoindoline-1,3-dione

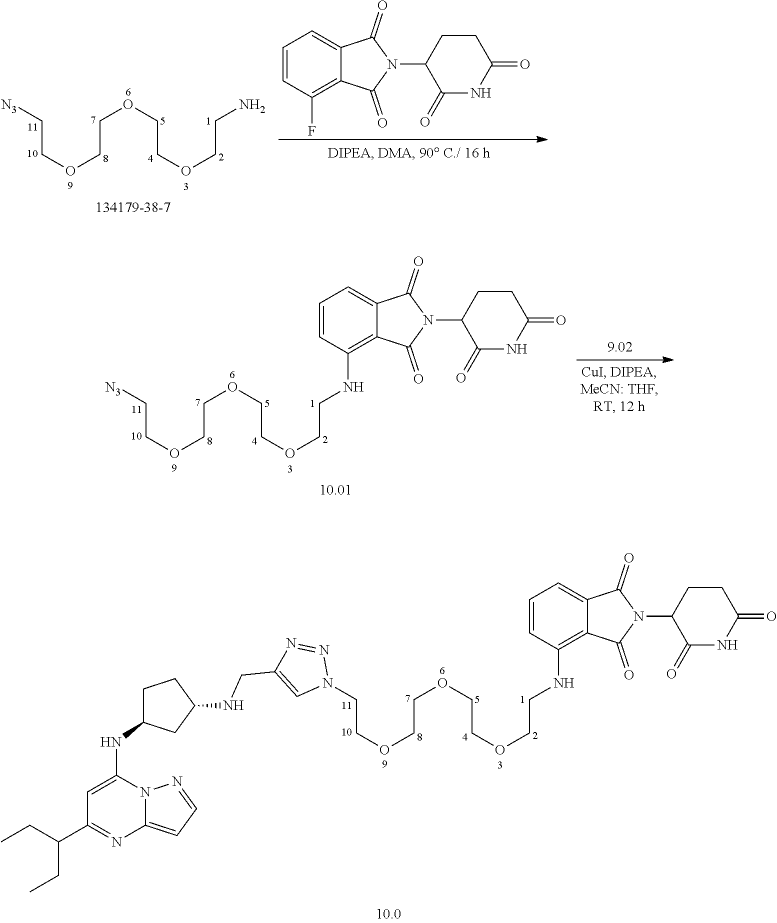

### Step-1: Synthesis of 4-[2-[2-[2-(2-azidoethoxy)ethoxy]ethoxy]ethylamino]-2-(2,6-dioxo-3-piperidyl)isoindoline-1,3-dione (Compound 10.01)

To a stirred solution of 2-[2-[2-(2-azidoethoxy)ethoxy]ethoxy]ethanamine (39.51 mg, 0.1800 mmol) in DMA (1 mL) was added 2-(2,6-dioxo-3-piperidyl)-4-fluoro-isoindoline-1,3-dione (50 mg, 0.1800 mmol) (Compound 5.01) and stirred for 16 h at 90° C. Reaction mixture was quenched with ice cold water and extracted with EtOAc (30 mL×2). The organic layer was washed with saturated brine solution (50 mL×1), dried over anhydrous Na₂SO₄, and evaporated under reduced pressure. The crude product was purified by Combiflash column chromatography (silica gel), eluent 70% EtOAc in Hexane to give 4-[2-[2-[2-(2-azidoethoxy)ethoxy]ethoxy]ethylamino]-2-(2,6-dioxo-3-piperidyl)isoindoline-1,3-dione (Compound 10.01) (45 mg, 52%) as a bright yellow solid. LCMS-ESI (pos.) m/z: 475.3 (M+1)⁺

### Step-2: Synthesis of 2-(2,6-dioxo-3-piperidyl)-4-[2-[2-[2-[2-[4-[[[(1S,3S)-3-[[5-(1-ethylpropyl)pyrazolo[1,5-a]pyrimidin-7-yl]amino]cyclopentyl]amino]methyl]triazol-1-yl]ethoxy]ethoxy]ethoxy]ethylamino]isoindoline-1,3-dione (Compound 10.0)

In an oven-dried 10 mL round-bottom flask, (1S,3S)—N3-[5-(1-ethylpropyl)pyrazolo[1,5-a]pyrimidin-7-yl]—N1-prop-2-ynyl-cyclopentane-1,3-diamine (25 mg, 0.0800 mmol) (Compound 9.02), 4-[2-[2-[2-(2-azidoethoxy)ethoxy]ethoxy]ethylamino]-2-(2,6-dioxo-3-piperidyl)isoindoline-1,3-dione (36.45 mg, 0.0800 mmol) (Compound 10.01), and CuI (8.62 mg, 0.0800 mmol) were added to a mixture of MeCN (1 mL) and THF (1 mL). Then DIPEA (0.01 mL, 0.0800 mmol) was added, and the reaction mixture was vigorously stirred at ambient temperature for 12 h under an argon atmosphere. The reaction mixture was concentrated to dryness, the residue was dissolved in EtOAc (1×20 mL) and washed with water (2×10 mL), followed by saturated brine solution (1×10 mL). The organic layer was separated and dried over anhydrous Na₂SO₄ and concentrated to dryness. The crude residue was purified by Prep-HPLC to give 2-(2,6-dioxo-3-piperidyl)-4-[2-[2-[2-[2-[4-[[[(1S,3S)-3-[[5-(1-ethylpropyl)pyrazolo[1,5-a]pyrimidin-7-yl]amino]cyclopentyl]amino]methyl]triazol-1-yl]ethoxy]ethoxy]ethoxy]ethylamino]isoindoline-1,3-dione (Compound 10.0) (4 mg, 7%). LCMS-ESI (pos.) m/z: 800.6 (M+1)⁺

¹H NMR (400 MHz, DMSO-d₆) δ 7.99 (d, J=2.2 Hz, 1H), 7.90 (s, 1H), 7.61-7.52 (m, 1H), 7.46 (d, J=7.8 Hz, 1H), 7.13 (d, J=8.5 Hz, 1H), 7.03 (d, J=7.0 Hz, 1H), 6.59 (t, J=6.0 Hz, 1H), 6.31 (d, J=2.2 Hz, 1H), 5.98 (s, 1H), 5.05 (dd, J=5.3, 12.8 Hz, 1H), 4.47 (t, J=5.2 Hz, 2H), 4.24-4.12 (m, 1H), 3.78 (t, J=5.1 Hz, 2H), 3.74-3.67 (m, 2H), 3.60 (t, J=5.4 Hz, 2H), 3.58-3.42 (m, 10H), 2.95-2.80 (m, 2H), 2.63-2.53 (m, 2H), 2.26-2.09 (m, 2H), 2.08-1.84 (m, 5H), 1.70-1.55 (m, 5H), 1.47-1.37 (m, 2H), 0.76 (t, J=7.3 Hz, 6H).

### Compound 11.0 2-(2,6-dioxo-3-piperidyl)-4-[2-[2-[2-[2-[[(1S,3S)-3-[[3-chloro-5-(1-ethylpropyl)pyrazolo[1,5-a]pyrimidin-7-yl]amino]cyclopentyl]amino]ethoxy]ethoxy]ethoxy]ethylamino]isoindoline-1,3-dione

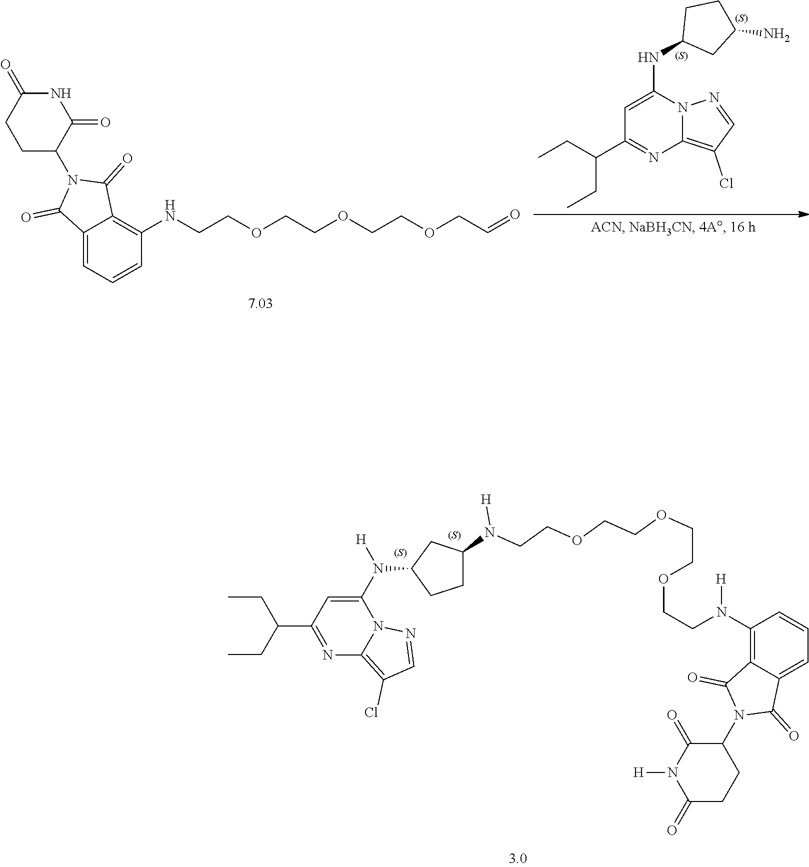

### Synthesis of 2-(2,6-dioxo-3-piperidyl)-4-[2-[2-[2-[2-[[(1S,3S)-3-[[3-chloro-5-(1-ethylpropyl)pyrazolo[1,5-a]pyrimidin-7-yl]amino]cyclopentyl]amino]ethoxy]ethoxy]ethoxy]ethylamino]isoindoline-1,3-dione (Compound 11.0)

(1S,3S)—N3-[3-chloro-5-(1-ethylpropyl)pyrazolo[1,5-a]pyrimidin-7-yl]cyclopentane-1,3-diamine (4.94 mg, 0.02 mmol) (CAS #2416874-47-8, prepared as described in US20200131189 A1), 2-[2-[2-[2-[[2-(2,6-dioxo-3-piperidyl)-1,3-dioxo-isoindolin-4-yl]amino]ethoxy]ethoxy]ethoxy]acetaldehyde (7.55 mg, 0.02 mmol) (Compound 7.03), and 4A MS in MeCN (3 mL) were stirred for 2 h at 0° C., followed by addition of NaBH₃CN (0.01 mL, 0.05 mmol). The reaction was allowed to warm to ambient temperature and stirred for 16 h. At this time, LCMS showed formation of product. The reaction mixture was concentrated under vacuum and was purified by Prep-HPLC purification, and desired fractions were lyophilized for 16 h to get 2-(2,6-dioxo-3-piperidyl)-4-[2-[2-[2-[2-[[(1S,3S)-3-[[3-chloro-5-(1-ethylpropyl)pyrazolo[1,5-a]pyrimidin-7 yl]amino]cyclopentyl]amino]ethoxy]ethoxy]ethoxy]ethylamino]isoindoline-1,3-dione (Compound 11.0) (3.17 mg, 26%). LCMS-ESI (pos.) m/z: 752.8 (M+1)⁺

¹H NMR (400 MHz, DMSO-d₆) δ 8.18-8.10 (m, 1H), 7.71 (t, J=8.3 Hz, 1H), 7.54 (s, 1H), 7.12-7.04 (m, 1H), 7.01 (dd, J=2.3, 7.0 Hz, 1H), 6.62-6.52 (m, 1H), 6.03-5.94 (m, 1H), 5.04 (dd, J=5.4, 13.1 Hz, 1H), 4.21-4.11 (m, 1H), 3.63-3.48 (m, 9H), 3.31-3.03 (m, 4H), 2.94-2.80 (m, 2H), 2.74-2.63 (m, 1H), 2.62-2.52 (m, 2H), 2.48-2.32 (m, 2H), 2.23-2.06 (m, 2H), 2.07-1.77 (m, 6H), 1.74-1.48 (m, 6H), 1.46-1.28 (m, 1H), 0.71 (t, J=7.6 Hz, 6H).

### Compound 12.0 2-(2,6-dioxo-3-piperidyl)-4-[[1-[2-[2-[2-[2-[[(1S,3S)-3-[[5-(1-ethylpropyl)pyrazolo[1,5-a]pyrimidin-7-yl]amino]cyclopentyl]amino]ethoxy]ethoxy]ethoxy]ethyl]triazol-4-yl]methylamino]isoindoline-1,3-dione

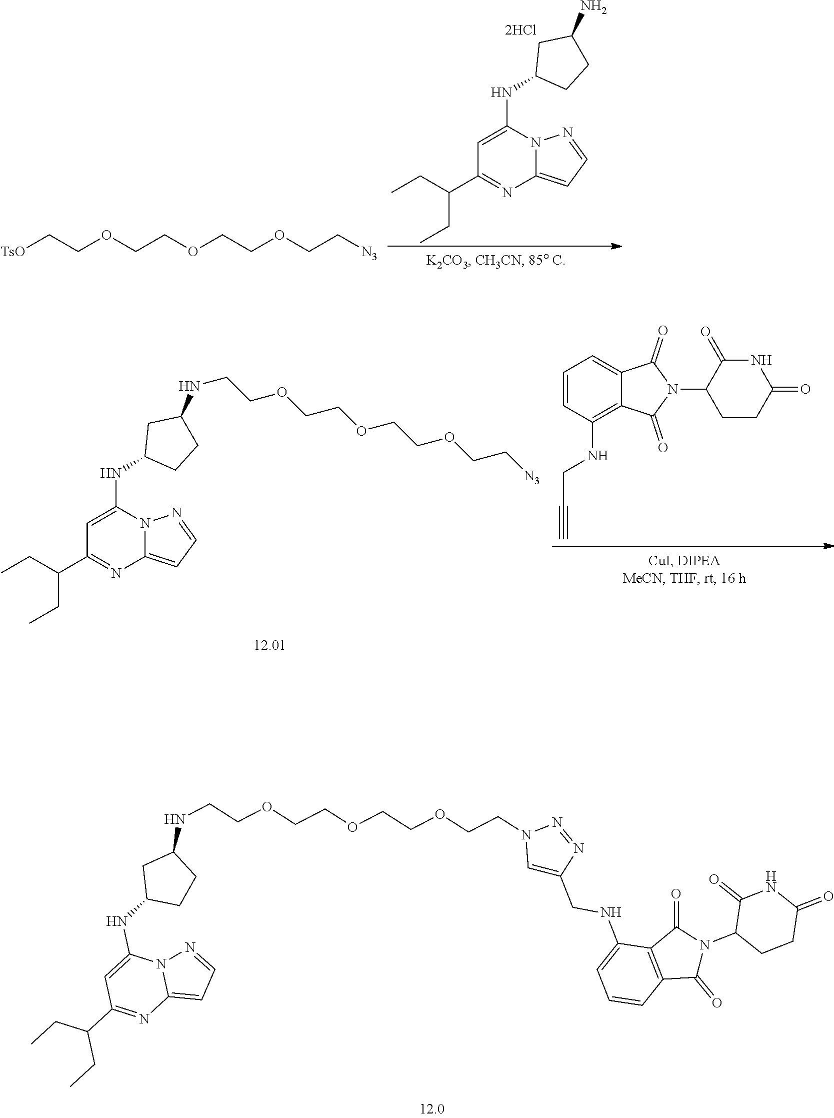

### Step-1: Synthesis of (1S,3S)—N¹-(2-(2-(2-(2-azidoethoxy)ethoxy)ethoxy)ethyl)—N³-(5-(pentan-3-yl)pyrazolo[1,5-a]pyrimidin-7-yl)cyclopentane-1,3-diamine (Compound 12.01)

To a stirred solution of (1S,3S)—N3-[5-(1-ethylpropyl)pyrazolo[1,5-a]pyrimidin-7-yl]cyclopentane-1,3-diamine (190 mg, 0.66 mmol) (CAS #2416873-83-9, prepared as described in US20200131189 A1) and 2-[2-[2-(2-azidoethoxy)ethoxy]ethoxy]ethyl 4-methylbenzenesulfonate (370.3 mg, 0.99 mmol) in MeCN (15 mL), was added potassium carbonate (456.16 mg, 3.31 mmol) and the reaction mixture was heated at 95° C. for 24 h. The reaction mixture was filtered, and the filtrate was concentrated in vacuo. The crude residue was purified by Combiflash column chromatography (silica gel), eluent 10% MeOH in DCM to give (1S,3S)—N¹-(2-(2-(2-(2-azidoethoxy)ethoxy)ethoxy)ethyl)—N³-(5-(pentan-3-yl)pyrazolo[1,5-a]pyrimidin-7-yl)cyclopentane-1,3-diamine (Compound 12.01) (130 mg, 40%). LCMS-ESI (pos.) m/z: 489.1 (M+1)⁺

### Step-2: Synthesis of 2-(2,6-dioxo-3-piperidyl)-4-[[1-[2-[2-[2-[2-[[(1S,3S)-3-[[5-(1-ethylpropyl)pyrazolo[1,5-a]pyrimidin-7-yl]amino]cyclopentyl]amino]ethoxy]ethoxy]ethoxy]ethyl]triazol-4-yl]methylamino]isoindoline-1,3-dione (Compound 12.0)

To a stirred solution of (1S,3S)—N1-[2-[2-[2-(2-azidoethoxy)ethoxy]ethoxy]ethyl]—N3-[5-(1-ethylpropyl)pyrazolo[1,5-a]pyrimidin-7-yl]cyclopentane-1,3-diamine (Compound 12.01) (10 mg, 0.02 mmol) in THF (1 mL) and MeCN (1 mL) was added CuI (2.3 mg, 0.02 mmol) at 0° C. and stirred for few minutes. 2-(2,6-dioxo-3-piperidyl)-4-(prop-2-ynylamino)isoindoline-1,3-dione (6.37 mg, 0.02 mmol) and DIPEA (0.01 mL, 0.02 mmol) were then added, and the reaction stirred for 16 h at ambient temperature. Progress of the reaction was monitored by crude LCMS. The reaction mixture was purified by Prep HPLC followed by lyophilization from MeCN/water to give 2-(2,6-dioxo-3-piperidyl)-4-[[1-[2-[2-[2-[2-[[(1S,3S)-3-[[5-(1-ethylpropyl)pyrazolo[1,5-a]pyrimidin-7-yl]amino]cyclopentyl]amino]ethoxy]ethoxy]ethoxy]ethyl]triazol-4-yl]methylamino]isoindoline-1,3-dione (Compound 12.0) (1.1 mg, 7%). LCMS-ESI (pos.) m/z: 800.3 (M+1)⁺

¹H NMR (400 MHz, DMSO-d₆) δ 7.99 (s, 2H), 7.61-7.53 (m, 1H), 7.46 (d, J=7.5 Hz, 1H), 7.17 (d, J=8.5 Hz, 1H), 7.05 (d, J=7.0 Hz, 2H), 6.31 (d, J=2.2 Hz, 1H), 5.97 (s, 1H), 5.05 (dd, J=5.4, 13.0 Hz, 1H), 4.59 (d, J=5.7 Hz, 2H), 4.48 (t, J=5.2 Hz, 2H), 4.17 (s, 1H), 3.78 (t, J=5.2 Hz, 2H), 3.59-3.40 (m, 10H), 2.91-2.82 (m, 1H1), 2.73-2.51 (m, 3H1), 2.47-2.39 (m, 2H1), 2.24-2.09 (m, 2H1), 2.06-1.80 (m, 6H), 1.76-1.54 (m, 5H), 1.50-1.29 (m, 1H), 0.76 (t, J=7.3 Hz, 6H).

### Compound 13.0 4-[2-[2-[2-[2-[2-[[(1S,3S)-3-[[3-chloro-5-(1-ethylpropyl)pyrazolo[1,5-a]pyrimidin-7-yl]amino]cyclopentyl]amino]ethoxy]ethoxy]ethoxy]ethoxy]ethylamino]-2-(2,6-dioxo-3-piperidyl)isoindoline-1,3-dione

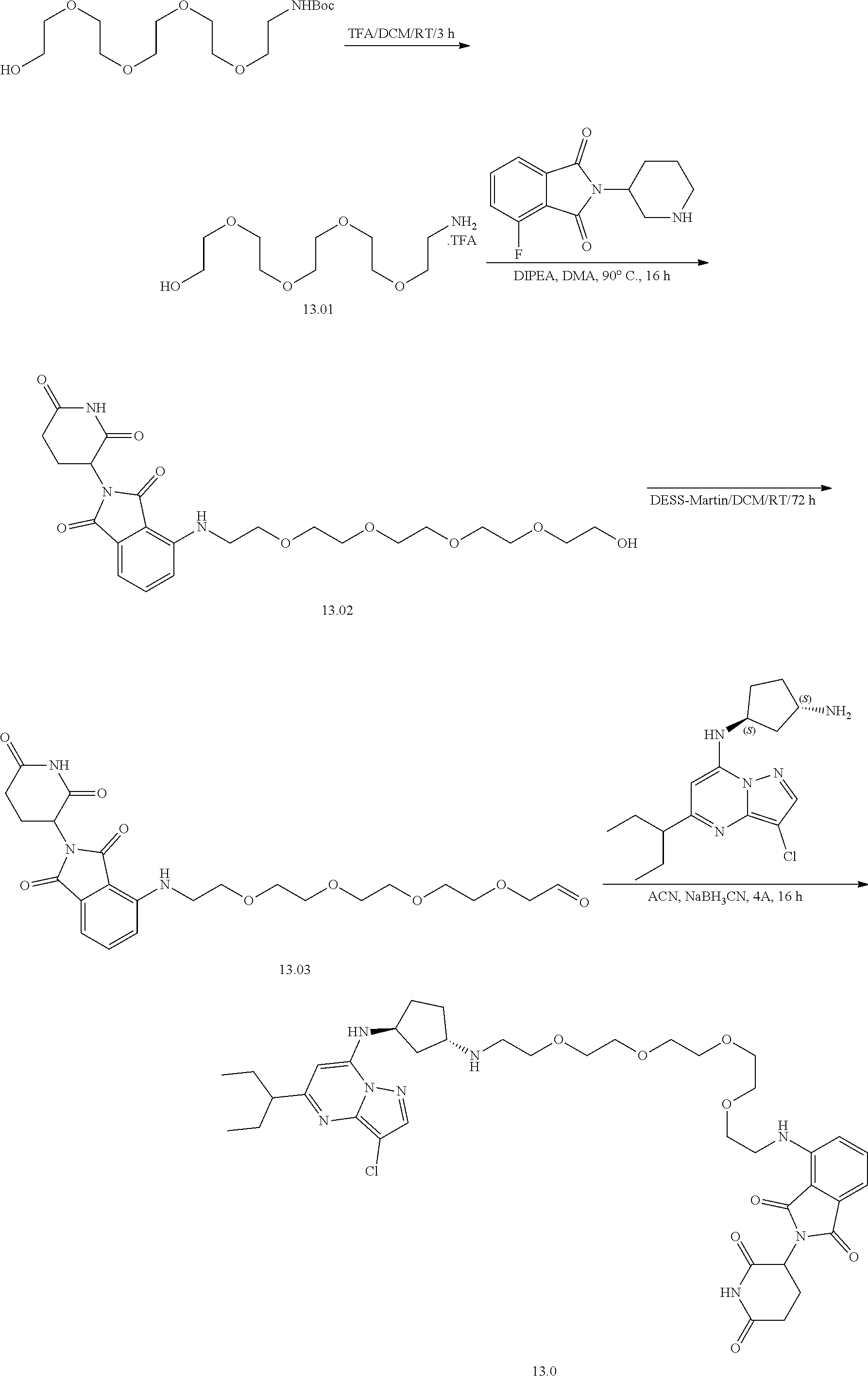

### Step-1: Synthesis of 2-[2-[2-[2-(2-hydroxyethoxy)ethoxy]ethoxy]ethoxy]ethylammonium; 2,2,2-trifluoroacetate (Compound 13.01)

To a solution of tert-butyl N-[2-[2-[2-[2-(2-hydroxyethoxy)ethoxy]ethoxy]ethoxy]ethyl]carbamate (320 mg, 0.95 mmol) [1404111-67-6] in DCM (6 mL) at 0° C., was added TFA (3 mL, 34.65 mmol) and stirred at ambient temperature for 3 h. After completion of the reaction, reaction mixture was concentrated in vacuo, co-evaporated with toluene, and lyophilized from MeCN:H₂O to give 2-[2-[2-[2-(2-hydroxyethoxy)ethoxy]ethoxy]ethoxy]ethylammonium 2,2,2-trifluoroacetate (Compound 13.01) (220 mg, 98%), which was directly used for the next reaction. LCMS-ESI (pos.) m/z: 238.4 (M+1)⁺

### Step-2: Synthesis of 2-(2,6-dioxo-3-piperidyl)-4-[2-[2-[2-[2-(2-hydroxyethoxy)ethoxy]ethoxy]ethoxy]ethylamino]isoindoline-1,3-dione (Compound 13.02)

To a stirred solution of 2-(2,6-dioxo-3-piperidyl)-4-fluoro-isoindoline-1,3-dione (Compound 5.01) (153.66 mg, 0.56 mmol) and 2-[2-[2-[2-(2-aminoethoxy)ethoxy]ethoxy]ethoxy]ethanol (Compound 13.01) (220 mg, 0.93 mmol) in DMA (2 mL), was added DIPEA (0.81 mL, 4.64 mmol) and heated at 90° C. for 16 h. After completion of the reaction, the reaction mixture was extracted with EtOAc and washed with water followed by saturated brine solution. The organic layer was separated and concentrated in vacuo. The crude product was purified by Combiflash column chromatography (silica gel), eluent 80% EtOAc in hexane to give 2-(2,6-dioxo-3-piperidyl)-4-[2-[2-[2-[2-(2-hydroxyethoxy)ethoxy]ethoxy]ethoxy]ethylamino]isoindoline-1,3-dione (Compound 13.02) (70 mg, 15%). LCMS-ESI (pos.) m/z: 493.9 (M+1)⁺

### Step-3: Synthesis of 2-[2-[2-[2-[2-[[2-(2,6-dioxo-3-piperidyl)-1,3-dioxo-isoindolin-4-yl]amino]ethoxy]ethoxy]ethoxy]ethoxy]acetaldehyde (Compound 13.03)

To a solution of 2-(2,6-dioxo-3-piperidyl)-4-[2-[2-[2-[2-(2-hydroxyethoxy)ethoxy]ethoxy]ethoxy]ethylamino]isoindoline-1,3-dione (60 mg, 0.12 mmol) (Compound 13.02) in DCM (5 mL) at 0° C. was added Dess-Martin periodinane (257.83 mg, 0.61 mmol), and the mixture stirred at ambient temperature for 72 h. After completion of the reaction, an aqueous Na₂S₂O₃ solution was added and stirred for 15 min. The reaction was extracted with DCM, washed with saturated NaHCO₃ solution, followed by water and saturated brine solution. The organic layer was dried over anhydrous Na₂SO₄ and concentrated in vacuo to give 2-[2-[2-[2-[2-[[2-(2,6-dioxo-3-piperidyl)-1,3-dioxo-isoindolin-4-yl]amino]ethoxy]ethoxy]ethoxy]ethoxy]acetaldehyde (Compound 13.03) (40 mg, 22%), which was directly used for the next reaction. LCMS-ESI (pos.) m/z: 492.51 (M+1)⁺

### Step-4: Synthesis of 4-[2-[2-[2-[2-[2-[[(1S,3S)-3-[[3-chloro-5-(1-ethylpropyl)pyrazolo[1,5-a]pyrimidin-7-yl]amino]cyclopentyl]amino]ethoxy]ethoxy]ethoxy]ethoxy]ethylamino]-2-(2,6-dioxo-3-piperidyl)isoindoline-1,3-dione (Compound 13.0)

To a solution of (1S,3S)—N¹-(3-chloro-5-(pentan-3-yl)pyrazolo[1,5-a]pyrimidin-7-yl)cyclopentane-1,3-diamine (CAS #2416874-47-8, prepared as described in US20200131189 A1) (20.96 mg, 0.07 mmol), and 2-[2-[2-[2-[2-[[2-(2,6-dioxo-3-piperidyl)-1,3-dioxo-isoindolin-4-yl]amino]ethoxy]ethoxy]ethoxy]ethoxy]acetaldehyde (Compound 13.03) (40 mg, 0.08 mmol) in MeCN (3 mL) at 0° C. was added 4A MS (100 mg) and stirred at ambient temperature for 2 h. Thereafter, NaBH₃CN (15.34 mg, 0.24 mmol) was added and stirred for 16 h. The reaction mixture was filtered, and the filtrate was concentrated in vacuo. The crude product was purified by Prep-HPLC and lyophilized from MeCN/water to give 4-[2-[2-[2-[2-[2-[[(1S,3S)-3-[[3-chloro-5-(1-ethylpropyl)pyrazolo[1,5-a]pyrimidin-7-yl]amino]cyclopentyl]amino]ethoxy]ethoxy]ethoxy]ethoxy]ethylamino]-2-(2,6-dioxo-3-piperidyl)isoindoline-1,3-dione (Compound 13.0) (6 mg, 9%). LCMS-ESI (pos.) m/z: 796.4 (M+1)⁺

¹H NMR (400 MHz, DMSO-d₆) δ 8.05 (s, 1H), 7.62-7.53 (m, 1H), 7.12 (d, J=8.5 Hz, 1H), 7.04 (d, J=7.0 Hz, 1H), 6.55-6.48 (m, 1H), 6.03 (s, 1H), 5.05-4.95 (m, 1H), 4.27-4.16 (m, 2H1), 3.67 (t, J=5.5 Hz, 2H), 3.63-3.50 (m, 9H), 3.54-3.44 (m, 3H), 3.29 (t, J=6.1 Hz, 1H), 2.72-2.63 (m, 2H), 2.65-2.51 (m, 3H), 2.29-2.17 (m, 2H), 2.13-1.94 (m, 2H), 1.96-1.87 (m, 2H), 1.87 (s, 2H), 1.82-1.48 (m, 5H), 1.49-1.35 (m, 2H), 0.84 (t, J=7.4 Hz, 6H).

### Compound 14.0 (2S,4R)-4-hydroxy-N-[[4-(4-methylthiazol-5-yl)phenyl]methyl]-1-[(2S)-3,3-dimethyl-2-[[2-[2-[[1-[2-[2-[2-[[(1S,3S)-3-[[5-(1-ethylpropyl)pyrazolo[1,5-a]pyrimidin-7-yl]amino]cyclopentyl]amino]ethoxy]ethoxy]ethyl]triazol-4-yl]methoxy]ethoxy]acetyl]amino]butanoyl]pyrrolidine-2-carboxamide

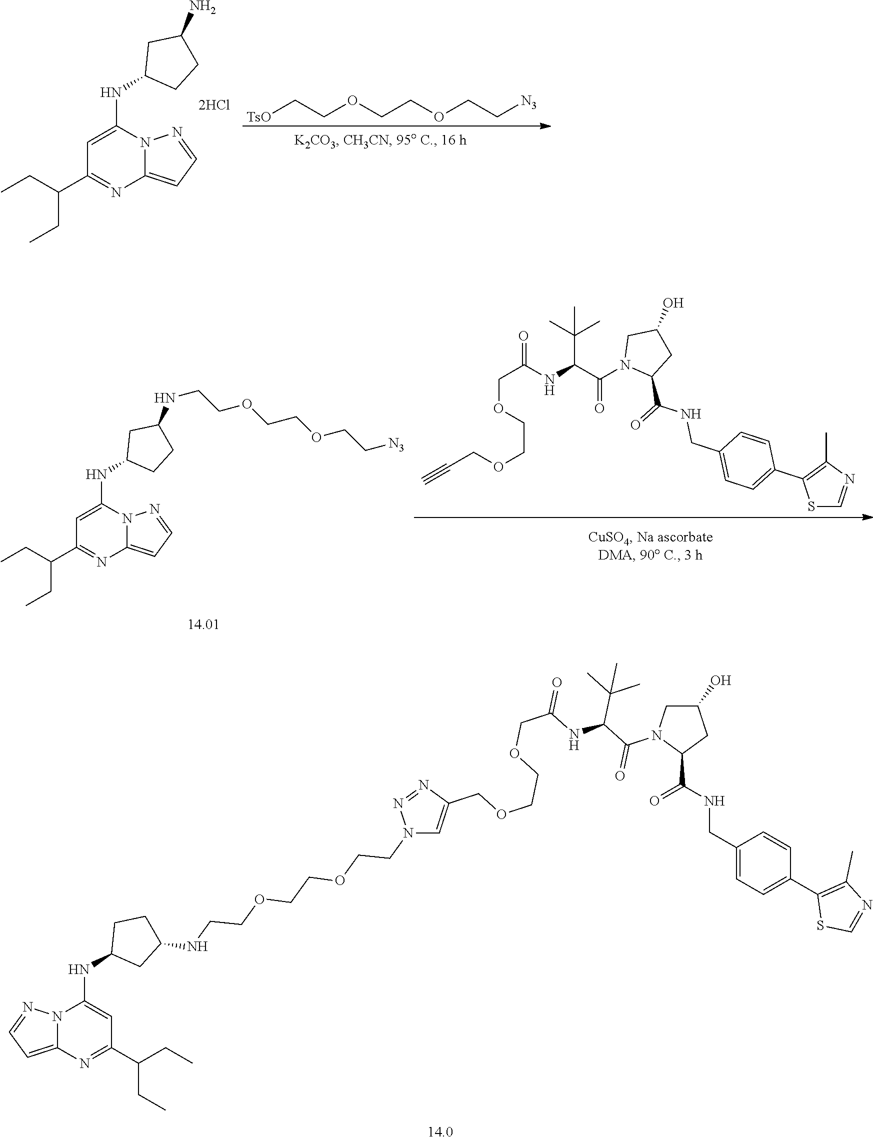 [SMILES: CCC(CC)c1cc(N[C@H]2CC[C@H](N)C2)n2nccc2n1 ; Cc1ccc(S(=O)(=O)OCCOCCOCCN=[N+]=[N-])cc1 ; CCC(CC)c1cc(N[C@H]2CC[C@H](NCCOCCOCCN=[N+]=[N-])C2)n2nccc2n1 ; C#CCOCCOCC(=O)N[C@H](C(=O)N1C[C@H](O)C[C@H]1C(=O)NCc1ccc(-c2scnc2C)cc1)C(C)(C)C ; CCC(CC)c1cc(N[C@H]2CC[C@H](NCCOCCOCCn3cc(COCCOCC(=O)N[C@H](C(=O)N4C[C@H](O)C[C@H]4C(=O)NCc4ccc(-c5scnc5C)cc4)C(C)(C)C)nn3)C2)n2nccc2n1]

### Synthesis of (1S,3S)—N1-[2-[2-(2-azidoethoxy)ethoxy]ethyl]—N3-[5-(1-ethylpropyl)pyrazolo[1,5-a]pyrimidin-7-yl]cyclopentane-1,3-diamine (Compound 14.01)

To a stirred solution of (1S,3S)—N3-[5-(1-ethylpropyl)pyrazolo[1,5-a]pyrimidin-7-yl]cyclopentane-1,3-diamine (175 mg, 0.61 mmol) (CAS #2416873-83-9, prepared as described in US20200131189 A1) and 2-[2-(2-azidoethoxy)ethoxy]ethyl 4-methylbenzenesulfonate Compound (250.69 mg, 0.76 mmol) in MeCN (10 mL) was added potassium carbonate (420.15 mg, 3.04 mmol), and the reaction mixture was heated at 95° C. for 24 h. Then the reaction mixture was filtered, and the filtrate was concentrated in vacuo. The crude product was purified by Combiflash column chromatography (silica gel), eluent 10% MeOH in DCM to give (1S,3S)—N1-[2-[2-(2-azidoethoxy)ethoxy]ethyl]—N3-[5-(1-ethylpropyl)pyrazolo[1,5-a]pyrimidin-7-yl]cyclopentane-1,3-diamine (Compound 14.01) (125 mg, 46%). LCMS-ESI (pos.) m/z: 445.4 (M+1)⁺

### Synthesis of (2S,4R)-4-hydroxy-N-[[4-(4-methylthiazol-5-yl)phenyl]methyl]-1-[(2S)-3,3-dimethyl-2-[[2-[2-[[1-[2-[2-[2-[[(1S,3S)-3-[[5-(1-ethylpropyl)pyrazolo[1,5-a]pyrimidin-7-yl]amino]cyclopentyl]amino]ethoxy]ethoxy]ethyl]triazol-4-yl]methoxy]ethoxy]acetyl]amino]butanoyl]pyrrolidine-2-carboxamide (Compound 14.0)

To a stirred solution of (2S,4R)-4-hydroxy-N-[[4-(4-methylthiazol-5-yl)phenyl]methyl]-1-[(2S)-3,3-dimethyl-2-[[2-(2-prop-2-ynoxyethoxy)acetyl]amino]butanoyl]pyrrolidine-2-carboxamide (25 mg, 0.04 mmol) (as described in Journal of Medicinal Chemistry (2018), 61(2), 453-461) in DMA (3 mL) were added (1S,3S)—N1-[2-[2-(2-azidoethoxy)ethoxy]ethyl]—N3-[5-(1-ethylpropyl)pyrazolo[1,5-a]pyrimidin-7-yl]cyclopentane-1,3-diamine (Compound 14.01) (19.47 mg, 0.04 mmol), copper sulphate (1.8 mg, 0.01 mmol), and sodium ascorbate (2.89 mg, 0.01 mmol), and the reaction mixture was heated at 90° C. for 3 h in a seal tube. Progress of the reaction was monitored by LCMS. The reaction mixture was directly purified by Prep HPLC followed by lyophilization to give (2S,4R)-4-hydroxy-N-[[4-(4-methylthiazol-5-yl)phenyl]methyl]-1-[(2S)-3,3-dimethyl-2-[[2-[2-[[1-[2-[2-[2-[[(1S,3S)-3-[[5-(1-ethylpropyl)pyrazolo[1,5-a]pyrimidin-7-yl]amino]cyclopentyl]amino]ethoxy]ethoxy]ethyl]triazol-4-yl]methoxy]ethoxy]acetyl]amino]butanoyl]pyrrolidine-2-carboxamide (Compound 14.0) (7 mg, 16%). LCMS-ESI (pos.) m/z: 1015.6 (M+1)⁺

¹H NMR (400 MHz, DMSO-d₆) δ 8.97 (s, 1H), 8.59 (t, J=6.0 Hz, 1H), 8.07 (s, 1H), 7.99 (d, J=2.3 Hz, 1H), 7.50-7.34 (m, 2H), 7.38 (s, 3H), 6.31 (d, J=2.3 Hz, 1H), 5.97 (s, 1H), 5.16 (s, 1H), 4.57 (q, J=2.8, 3.2 Hz, 3H), 4.53-4.39 (m, 3H), 4.40-4.21 (m, 3H), 4.21-4.11 (m, 1H), 3.95 (s, 2H), 3.80 (t, J=5.2 Hz, 2H), 3.71-3.54 (m, 6H), 3.55-3.48 (m, 2H), 3.50-3.42 (m, 2H), 3.42 (t, J=5.6 Hz, 2H), 3.23 (t, J=6.0 Hz, 1H), 2.64 (t, J=5.7 Hz, 2H), 2.42 (s, 3H), 2.22-2.10 (m, 1H), 2.11-2.00 (m, 1H), 1.99-1.83 (m, 6H), 1.72-1.55 (m, 6H), 1.46-1.31 (m, 1H), 0.93 (s, 9H), 0.76 (t, J=7.3 Hz, 6H).

### Compound 15.0 5-[[1-[2-[2-[2-[[(1S,3S)-3-[[3-chloro-5-(1-ethylpropyl)pyrazolo[1,5-a]pyrimidin-7-yl]amino]cyclopentyl]amino]ethoxy]ethoxy]ethyl]triazol-4-yl]methylamino]-2-(2,6-dioxo-3-piperidyl)isoindoline-1,3-dione

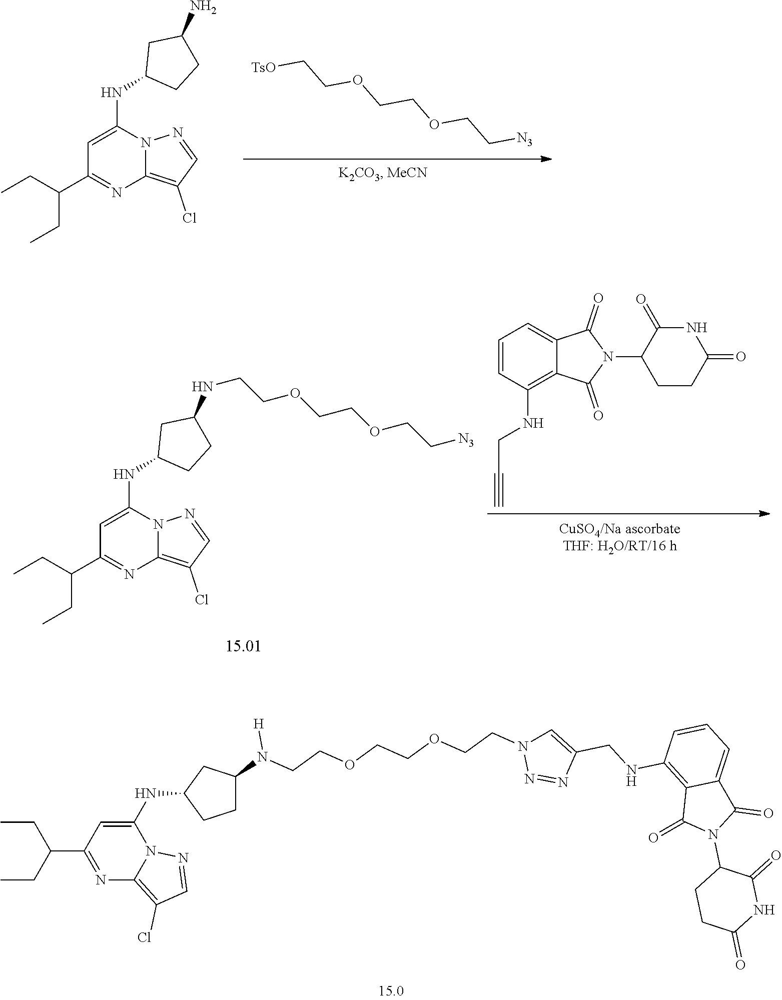

### Step-1: Synthesis of (1S,3S)—N1-[2-[2-(2-azidoethoxy)ethoxy]ethyl]—N3-[3-chloro-5-(1-ethylpropyl)pyrazolo[1,5-a]pyrimidin-7-yl]cyclopentane-1,3-diamine (Compound 15.01)

To a stirred solution of [(1S,3S)-3-[[3-chloro-5-(1-ethylpropyl)pyrazolo[1,5-a]pyrimidin-7-yl]amino]cyclopentyl]ammonium chloride (156.65 mg, 0.44 mmol) (CAS #2416874-47-8, prepared as described in US20200131189 A1) in MeCN (10 mL), were added 2-[2-(2-azidoethoxy)ethoxy]ethyl 4-methylbenzenesulfonate (180 mg, 0.55 mmol) and K₂CO₃ (603.33 mg, 4.37 mmol), and the reaction mixture refluxed for 48 h. The reaction mixture was filtered over celite, and the filtrate was concentrated in vacuo. The crude product was purified by Combiflash column chromatography (silica gel), eluent 5% MeOH in DCM to give (1S,3S)—N1-[2-[2-(2-azidoethoxy)ethoxy]ethyl]—N3-[3-chloro-5-(1-ethylpropyl)pyrazolo[1,5-a]pyrimidin-7-yl]cyclopentane-1,3-diamine (Compound 15.01) (120 mg, 44%). LCMS-ESI (pos.) m/z: 478.9 (M+1)⁺

### Step-2: Synthesis of 5-[[1-[2-[2-[2-[[(1S,3S)-3-[[3-chloro-5-(1-ethylpropyl)pyrazolo[1,5-a]pyrimidin-7-yl]amino]cyclopentyl]amino]ethoxy]ethoxy]ethyl]triazol-4-yl]methylamino]-2-(2,6-dioxo-3-piperidyl)isoindoline-1,3-dione (Compound 15.0)

To a solution of (1S,3S)—N1-[2-[2-(2-azidoethoxy)ethoxy]ethyl]—N3-[3-chloro-5-(1-ethylpropyl)pyrazolo[1,5-a]pyrimidin-7-yl]cyclopentane-1,3-diamine (Compound 15.01) (53 mg, 0.11 mmol) in THF (2 mL) were added 2-(2,6-dioxo-3-piperidyl)-4-(prop-2-ynylamino)isoindoline-1,3-dione (34.44 mg, 0.11 mmol), CuSO₄ (3.53 mg, 0.02 mmol), sodium ascorbate (4.38 mg, 0.02 mmol), and water (0.2 mL), and the reaction mixture stirred at ambient temperature for 16 h. The reaction mixture was evaporated in vacuo, and the residue purified by Prep-HPLC followed by lyophilization from MeCN:H₂O to give 5-[[1-[2-[2-[2-[[(1S,3S)-3-[[3-chloro-5-(1-ethylpropyl)pyrazolo[1,5-a]pyrimidin-7-yl]amino]cyclopentyl]amino]ethoxy]ethoxy]ethyl]triazol-4-yl]methylamino]-2-(2,6-dioxo-3-piperidyl)isoindoline-1,3-dione (Compound 15.0) (25.18 mg, 28%). LCMS-ESI (pos.) m/z: 790.4 (M+1)⁺

¹H NMR (400 MHz, DMSO-d₆) δ 8.05 (s, 1H), 7.95 (s, 1H), 7.62-7.53 (m, 1H), 7.20 (d, J=8.6 Hz, 1H), 7.16-7.08 (m, 1H), 7.06 (d, J=7.1 Hz, 1H), 6.90-6.82 (m, 1H), 6.03 (s, 1H), 5.00 (dd, J=5.6, 12.5 Hz, 1H), 4.61 (d, J=6.0 Hz, 2H), 4.50 (t, J=5.4 Hz, 2H), 4.21 (q, J=6.9 Hz, 1H), 3.85 (t, J=5.4 Hz, 2H), 3.58-3.40 (m, 6H), 3.33-3.23 (m, 1H), 2.90-2.80 (m, 2H), 2.69-2.50 (m, 4H), 2.28-2.16 (m, 1H), 2.13-1.92 (m, 3H), 1.96-1.87 (m, 4H), 1.80-1.60 (m, 4H), 1.48-1.34 (m, 1H), 0.84 (t, J=7.4 Hz, 6H).

### Compound 16.0 5-[[1-[2-[2-[2-[2-[[(1S,3S)-3-[[3-chloro-5-(1-ethylpropyl)pyrazolo[1,5-a]pyrimidin-7-yl]amino]cyclopentyl]amino]ethoxy]ethoxy]ethoxy]ethyl]triazol-4-yl]methylamino]-2-(2,6-dioxo-3-piperidyl)isoindoline-1,3-dione

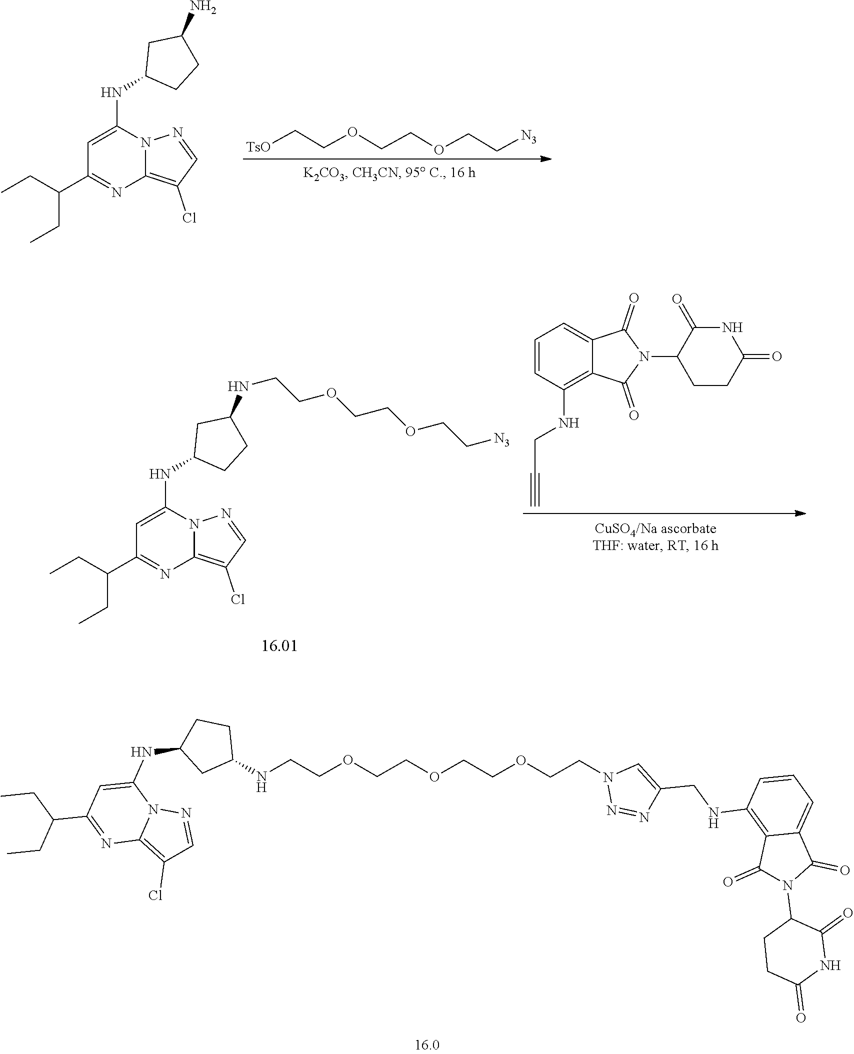

### Step-1: Synthesis of (1S,3S)—N1-[2-[2-[2-(2-azidoethoxy)ethoxy]ethoxy]ethyl]—N3-[3-chloro-5-(1-ethylpropyl)pyrazolo[1,5-a]pyrimidin-7-yl]cyclopentane-1,3-diamine (Compound 16.01)

To a stirred solution of [(1S,3S)-3-[[3-chloro-5-(1-ethylpropyl)pyrazolo[1,5-a]pyrimidin-7-yl]amino]cyclopentyl]ammonium chloride (153.53 mg, 0.43 mmol) (CAS #2416874-47-8, prepared using the procedure as described in US20200131189 A1) in MeCN (10 mL) were added 2-[2-[2-(2-azidoethoxy)ethoxy]ethoxy]ethyl 4-methylbenzenesulfonate (200 mg, 0.54 mmol) and K₂CO₃ (591.29 mg, 4.28 mmol), and the reaction mixture refluxed for 48 h. The reaction mixture was filtered over celite, and the filtrate was concentrated in vacuo. The crude product was purified by Combiflash column chromatography (silica gel), eluent 5% MeOH in DCM to give (1S,3S)—N1-[2-[2-[2-(2-azidoethoxy)ethoxy]ethoxy]ethyl]—N3-[3-chloro-5-(1-ethylpropyl)pyrazolo[1,5-a]pyrimidin-7-yl]cyclopentane-1,3-diamine (Compound 16.01) (110 mg, 37%). LCMS-ESI (pos.) m/z: 523.3 (M+1)⁺

### Step-2: Synthesis of 5-[[1-[2-[2-[2-[2-[[(1S,3S)-3-[[3-chloro-5-(1-ethylpropyl)pyrazolo[1,5-a]pyrimidin-7-yl]amino]cyclopentyl]amino]ethoxy]ethoxy]ethoxy]ethyl]triazol-4-yl]methylamino]-2-(2,6-dioxo-3-piperidyl)isoindoline-1,3-dione (Compound 16.0)

To a solution of (1S,3S)—N1-[2-[2-[2-(2-azidoethoxy)ethoxy]ethoxy]ethyl]—N3-[3-chloro-5-(1-ethylpropyl)pyrazolo[1,5-a]pyrimidin-7-yl]cyclopentane-1,3-diamine (Compound 16.01) (48 mg, 0.09 mmol) in THF (2 mL) were added 2-(2,6-dioxo-3-piperidyl)-4-(prop-2-ynylamino)isoindoline-1,3-dione (28.57 mg, 0.09 mmol), CuSO₄ (2.93 mg, 0.02 mmol), sodium ascorbate (3.64 mg, 0.02 mmol), and water (0.2 mL), and the reaction mixture stirred at ambient temperature for 16 h. The reaction mixture was evaporated in vacuo, and the residue was purified by Prep-HPLC followed by lyophilization from MeCN:H₂O to give 5-[[1-[2-[2-[2-[2-[[(1S,3S)-3-[[3-chloro-5-(1-ethylpropyl)pyrazolo[1,5-a]pyrimidin-7-yl]amino]cyclopentyl]amino]ethoxy]ethoxy]ethoxy]ethyl]triazol-4-yl]methylamino]-2-(2,6-dioxo-3-piperidyl)isoindoline-1,3-dione (Compound 16.0) (16 mg, 21%). LCMS-ESI (pos.) m/z: 834.4 (M+1)⁺

¹H NMR (400 MHz, DMSO-d₆) δ 8.05 (s, 1H), 7.95 (s, 1H), 7.62-7.53 (m, 1H), 7.20 (d, J=8.5 Hz, 1H), 7.18-7.07 (m, 1H), 7.06 (d, J=7.1 Hz, 1H), 6.89-6.82 (m, 1H), 6.03 (s, 1H), 5.00 (dd, J=5.5, 12.5 Hz, 1H), 4.61 (d, J=5.7 Hz, 2H), 4.50 (t, J=5.5 Hz, 2H), 4.27-4.14 (m, 1H), 3.84 (t, J=5.5 Hz, 2H), 3.57-3.40 (m, 9H), 3.32-3.24 (m, 1H), 2.89-2.80 (m, 1H), 2.72-2.51 (m, 5H), 2.30-2.14 (m, 2H), 2.14-1.94 (m, 2H), 1.95-1.87 (m, 2H), 1.80 (s, 3H), 1.77-1.62 (m, 4H), 1.46-1.36 (m, 1H), 0.84 (t, J=7.4 Hz, 6H).

### Compound 17.0 2-(2,6-dioxo-3-piperidyl)-4-[2-[4-[2-[[(1S,3S)-3-[[5-(1-ethylpropyl)pyrazolo[1,5-a]pyrimidin-7-yl]amino]cyclopentyl]amino]-1-methyl-ethyl]piperazin-1-yl]ethylamino]isoindoline-1,3-dione

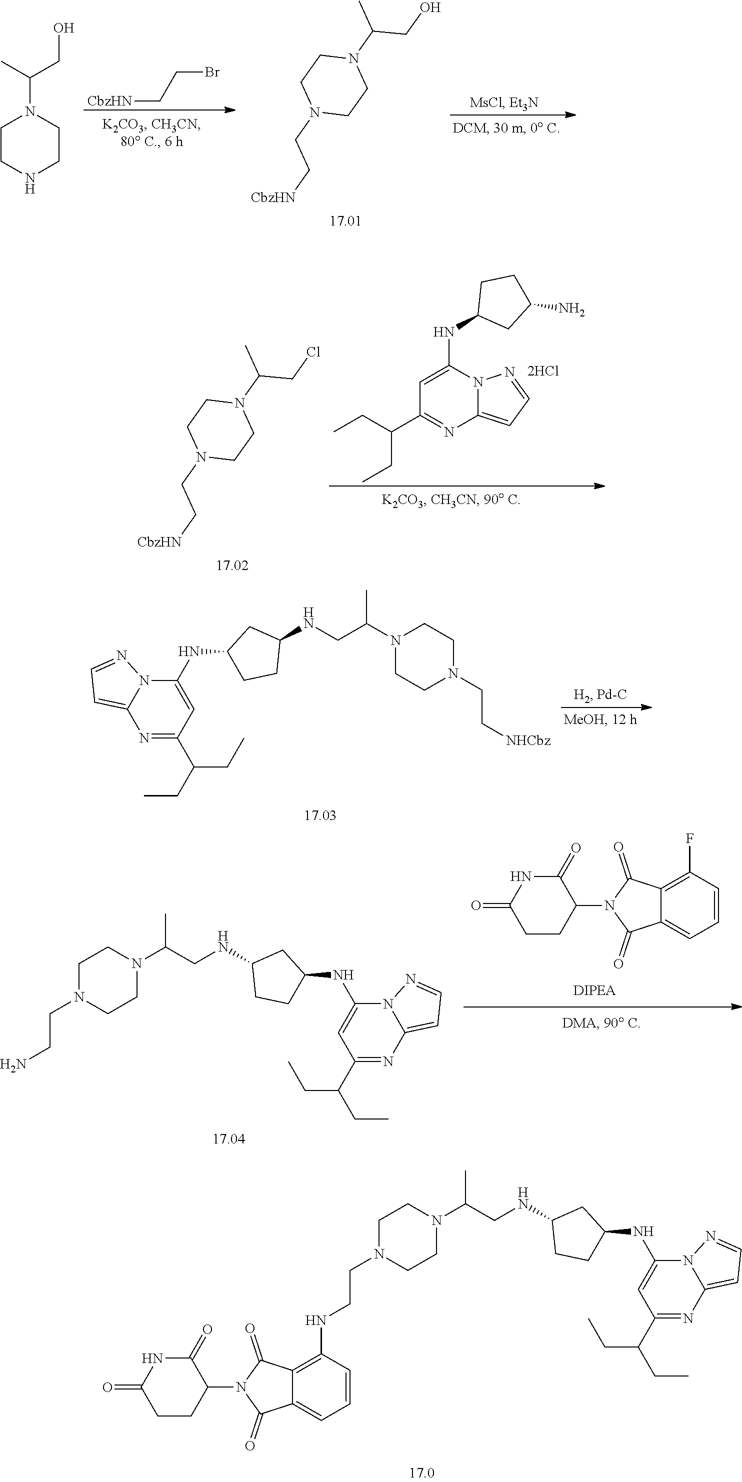

### Step-1: Synthesis of benzyl N-[2-[4-(2-hydroxy-1-methyl-ethyl)piperazin-1-yl]ethyl]carbamate (Compound 17.01)

To a stirred solution of benzyl N-(2-bromoethyl)carbamate (805.42 mg, 3.12 mmol) in MeCN (15 mL), were added K₂CO₃ (574.16 mg, 4.16 mmol) and 2-piperazin-1-ylpropan-1-ol (300 mg, 2.08 mmol), and the reaction mixture was heated at 80° C. for 12 h. After completion of reaction as monitored by LCMS, the reaction mixture was filtered through celite bed, and the celite bed was washed with EtOAc. The filtrate was dried over anhydrous Na₂SO₄ and evaporated in vacuo. The crude product was purified by neutral alumina using 5% MeOH in DCM to give the benzyl N-[2-[4-(2-hydroxy-1-methyl-ethyl)piperazin-1-yl]ethyl]carbamate (Compound 17.01) (500 mg, 75%). LCMS-ESI (pos.) m/z: 322.1 (M+1)⁺

### Step-2: Synthesis of benzyl N-[2-[4-(2-chloro-1-methyl-ethyl)piperazin-1-yl]ethyl]carbamate (Compound 17.02)

To a stirred solution of benzyl N-[2-[4-(2-hydroxy-1-methyl-ethyl)piperazin-1-yl]ethyl]carbamate (Compound 17.01) (345 mg, 1.07 mmol) in DCM (10 mL) at 0° C. were added triethylamine (0.46 mL, 3.22 mmol) and methanesulfonyl chloride (0.12 mL, 1.5 mmol), and the reaction mixture was stirred for 30 min at ambient temperature. After completion of reaction as monitored by TLC, DCM (25 mL) was added to the reaction mixture, and the organic layer was washed with saturated NaHCO₃ solution followed by saturated brine solution. The organic layer was dried over anhydrous Na₂SO₄ and evaporated in vacuo to give the benzyl N-[2-[4-(2-chloro-1-methyl-ethyl)piperazin-1-yl]ethyl]carbamate (Compound 17.02) (350 mg of crude), which was used in the next step without purification. LCMS-ESI (pos.) m/z: 340.2 (M+1)⁺

### Step-3: Synthesis of benzyl N-[2-[4-[2-[[(1S,3S)-3-[[5-(1-ethylpropyl)pyrazolo[1,5-a]pyrimidin-7-yl]amino]cyclopentyl]amino]-1-methyl-ethyl]piperazin-1-yl]ethyl]carbamate (Compound 17.03)

To a stirred solution of (1S,3S)—N3-[5-(1-ethylpropyl)pyrazolo[1,5-a]pyrimidin-7-yl]cyclopentane-1,3-diamine dihydrochloride (160 mg, 0.44 mmol) (CAS #2416873-83-9, prepared using the procedure as described in US20200131189 A1) and benzyl N-[2-[4-(2-chloro-1-methyl-ethyl)piperazin-1-yl]ethyl]carbamate (196.18 mg crude, approx. 0.58 mmol) in MeCN (10 mL) was added potassium carbonate (612.77 mg, 4.44 mmol), and the reaction mixture was heated at 95° C. for 16 h. The reaction mixture was filtered through celite and the filtrate was concentrated in vacuo. The crude product was purified by prep-HPLC to give benzyl N-[2-[4-[2-[[(1S,3S)-3-[[5-(1-ethylpropyl)pyrazolo[1,5-a]pyrimidin-7-yl]amino]cyclopentyl]amino]-1-methyl-ethyl]piperazin-1-yl]ethyl]carbamate (Compound 17.03) (95 mg, 36%). LCMS-ESI (pos.) m/z: 591.3 (M+1)⁺

### Step-4: Synthesis of (1S,3S)—N1-[2-[4-(2-aminoethyl)piperazin-1-yl]propyl]—N3-[5-(1-ethylpropyl)pyrazolo[1,5-a]pyrimidin-7-yl]cyclopentane-1,3-diamine (Compound 17.04)

To a stirred solution of benzyl N-[2-[4-[2-[[(1S,3S)-3-[[5-(1-ethylpropyl)pyrazolo[1,5-a]pyrimidin-7-yl]amino]cyclopentyl]amino]-1-methyl-ethyl]piperazin-1-yl]ethyl]carbamate (Compound 17.03) (95 mg, 0.16 mmol) in methanol (5 mL) was added 10% Pd—C (3.39 mg, 0.03 mmol). The reaction mixture was stirred under hydrogen atmosphere for 16 h at ambient temperature. After completion of reaction as monitored by LCMS, the reaction mixture was filtered through celite bed and the celite was washed with methanol. The filtrate was concentrated in vacuo to give (1S,3S)—N1-[2-[4-(2-aminoethyl)piperazin-1-yl]propyl]—N3-[5-(1-ethylpropyl)pyrazolo[1,5-a]pyrimidin-7-yl]cyclopentane-1,3-diamine (Compound 17.04) (60 mg crude, 82%). LCMS-ESI (pos.) m/z: 457.52 (M+1)⁺

### Step-5: Synthesis of 2-(2,6-dioxo-3-piperidyl)-4-[2-[4-[2-[[(1S,3S)-3-[[5-(1-ethylpropyl)pyrazolo[1,5-a]pyrimidin-7-yl]amino]cyclopentyl]amino]-1-methyl-ethyl]piperazin-1-yl]ethylamino]isoindoline-1,3-dione (Compound 17.0)

To a stirred solution of (1S,3S)—N1-[2-[4-(2-aminoethyl)piperazin-1-yl]propyl]—N3-[5-(1-ethylpropyl)pyrazolo[1,5-a]pyrimidin-7-yl]cyclopentane-1,3-diamine (Compound 17.04) (60 mg crude, approx. 0.13 mmol) and 2-(2,6-dioxo-3-piperidyl)-4-fluoro-isoindoline-1,3-dione (Compound 5.01) (30 mg, 0.11 mmol) in DMA (2 mL), was added DIPEA (0.06 mL, 0.33 mmol) and the reaction mixture was heated at 90° C. for 12 h. After completion of reaction as monitored by LCMS, the reaction mixture was poured into ice-cold water, extracted with EtOAc (2×20 mL), and washed with saturated brine solution. The organic layer was the dried over anhydrous Na₂SO₄ and concentrated in vacuo to give the crude. The crude product was purified by prep-HPLC using ammonium acetate buffer/MeCN mobile phase followed by lyophilization using MeCN/H₂O to give 2-(2,6-dioxo-3-piperidyl)-4-[2-[4-[2-[[(1S,3S)-3-[[5-(1-ethylpropyl)pyrazolo[1,5-a]pyrimidin-7-yl]amino]cyclopentyl]amino]-1-methyl-ethyl]piperazin-1-yl]ethylamino]isoindoline-1,3-dione (Compound 17.0) (5 mg, 6%). LCMS-ESI (pos.) m/z: 713.5 (M+1)⁺

¹H NMR (400 MHz, DMSO-d₆, D₂O) δ 7.99 (d, J=2.2 Hz, 1H), 7.58 (t, J=7.7 Hz, 1H), 7.07 (d, J=8.5 Hz, 1H), 7.02 (d, J=7.1 Hz, 1H), 6.30 (d, J=2.2 Hz, 1H), 5.96 (d, J=8.1 Hz, 1H), 5.02 (d, J=12.0 Hz, 1H), 4.35-3.95 (m, 1H), 3.39-3.20 (m, 5H), 2.88-2.79 (m, 2H), 2.65-2.56 (m, 2H), 2.46-2.41 (m, 8H), 2.27-2.08 (m, 2H), 2.09-1.85 (m, 5H), 1.74-1.53 (m, 8H), 0.92 (dd, J=6.2, 17.1 Hz, 2H), 0.74 (t, J=7.3 Hz, 6H).

### Compound 18.0 (2S,4R)-1-[(2S)-2-[[2-[2-[4-[2-[[(1S,3S)-3-[[5-(1-ethylpropyl)pyrazolo[1,5-a]pyrimidin-7-yl]amino]cyclopentyl]amino]ethyl]piperazin-1-yl]ethoxy]acetyl]amino]-3,3-dimethyl-butanoyl]-4-hydroxy-N-[[4-(4-methylthiazol-5-yl)phenyl]methyl]pyrrolidine-2-carboxamide

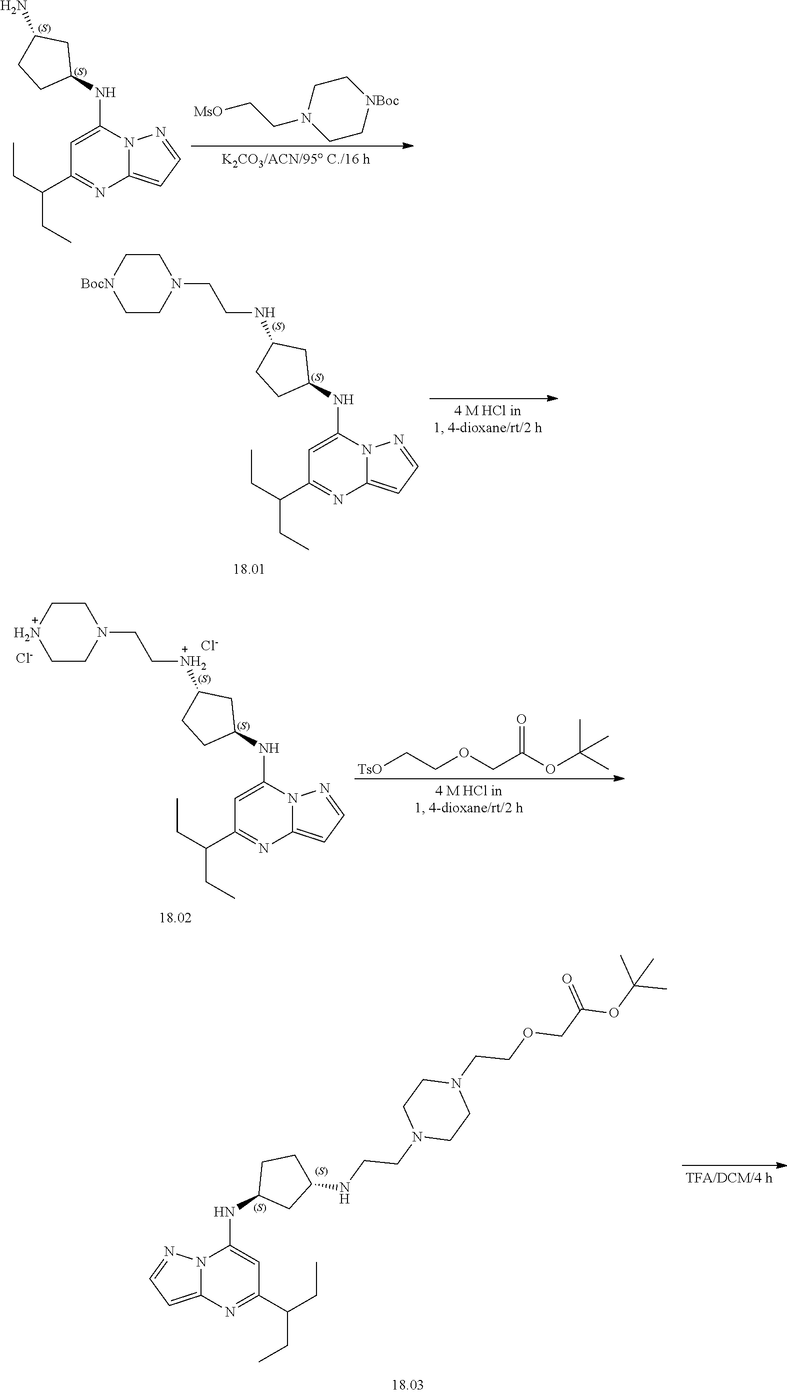

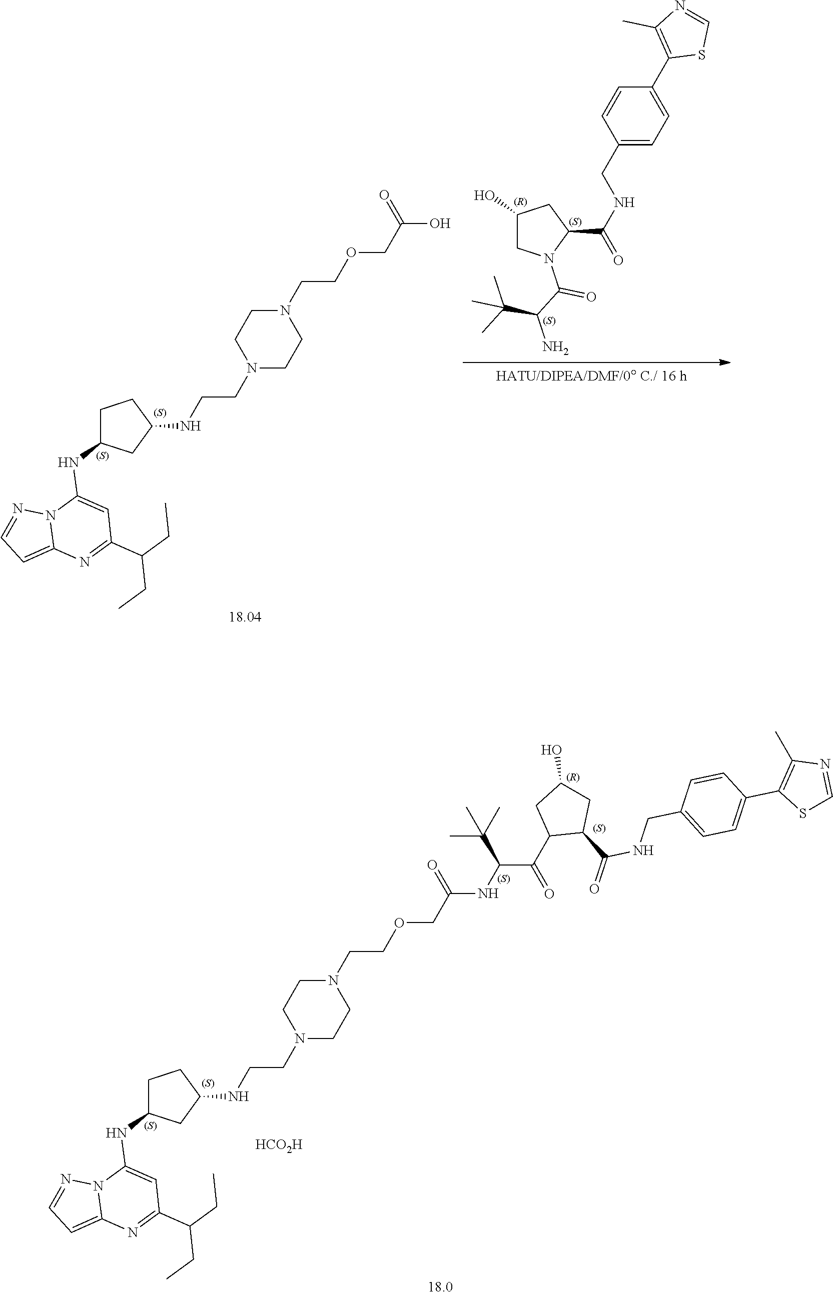

### Step-1: Synthesis of tert-butyl 4-[2-[[(1S,3S)-3-[[5-(1-ethylpropyl)pyrazolo[1,5-a]pyrimidin-7-yl]amino]cyclopentyl]amino]ethyl]piperazine-1-carboxylate (Compound 18.01)

To a stirred suspension of potassium carbonate (264.07 mg, 2.61 mmol) in MeCN (10 mL) at ambient temperature were added (1S,3S)—N3-[5-(1-ethylpropyl)pyrazolo[1,5-a]pyrimidin-7-yl]cyclopentane-1,3-diamine (300 mg, 1.04 mmol) (CAS #2416873-83-9, prepared using the procedure as described in US20200131189 A1) and tert-butyl 4-(2-methylsulfonyloxyethyl)piperazine-1-carboxylate (482.87 mg, 1.57 mmol). The reaction mixture was refluxed at 95° C. for 16 h. After completion of the reaction as monitored by TLC, it was filtered through a sintered funnel and evaporated to dryness in vacuo. The crude residue was purified by Combiflash column chromatography (amine silica), eluent 5% MeOH in DCM, and the desired fractions were evaporated in vacuo to give tert-butyl 4-[2-[[(1S,3S)-3-[[5-(1-ethylpropyl)pyrazolo[1,5-a]pyrimidin-7-yl]amino]cyclopentyl]amino]ethyl]piperazine-1-carboxylate (Compound 18.01) (200 mg, 38%). LCMS-ESI (pos.) m/z: 500.5 (M+1)⁺

### Step-2: Synthesis of 2-piperazin-4-ium-1-ylethyl-[(1S,3S)-3-[[5-(1-ethylpropyl)pyrazolo[1,5-a]pyrimidin-7-yl]amino]cyclopentyl]ammonium dichloride (Compound 18.02)

tert-butyl 4-[2-[[(1S,3S)-3-[[5-(1-ethylpropyl)pyrazolo[1,5-a]pyrimidin-7-yl]amino]cyclopentyl]amino]ethyl]piperazine-1-carboxylate (Compound 18.01) (230 mg, 0.46 mmol) was taken in a round bottom flask and 4 M HCl in 1,4-dioxane (5 mL) was added. The reaction mixture was stirred for 3 h at ambient temperature. The reaction mixture was concentrated to dryness in vacuo and washed with n-pentane to give 2-piperazin-4-ium-1-ylethyl-[(1S,3S)-3-[[5-(1-ethylpropyl)pyrazolo[1,5-a]pyrimidin-7-yl]amino]cyclopentyl]ammonium dichloride (Compound 18.02) (216 mg, 99%). LCMS-ESI (pos.) m/z: 400.4 (M+1)⁺

### Step-3: Synthesis of tert-butyl 2-[2-[4-[2-[[(1S,3S)-3-[[5-(1-ethylpropyl)pyrazolo[1,5-a]pyrimidin-7-yl]amino]cyclopentyl]amino]ethyl]piperazin-1-yl]ethoxy]acetate (Compound 18.03)

To a stirred suspension of potassium carbonate (122.51 mg, 1.21 mmol) in MeCN (10 mL) at ambient temperature were added tert-butyl 2-[2-(p-tolylsulfonyloxy)ethoxy]acetate (123.07 mg, 0.37 mmol) and 2-piperazin-4-ium-1-ylethyl-[(1S,3S)-3-[[5-(1-ethylpropyl)pyrazolo[1,5-a]pyrimidin-7-yl]amino]cyclopentyl]ammonium dichloride (Compound 18.02) (160 mg, 0.34 mmol). The reaction was refluxed at 90° C. for 16 h. After completion of the reaction as monitored by TLC, it was filtered through a sintered funnel and evaporated to dryness in vacuo. The crude residue was purified by Combiflash column chromatography (silica gel) in 5% MeOH in DCM and the desired fractions were evaporated in vacuo to give tert-butyl 2-[2-[4-[2-[[(1S,3S)-3-[[5-(1-ethylpropyl)pyrazolo[1,5-a]pyrimidin-7-yl]amino]cyclopentyl]amino]ethyl]piperazin-1-yl]ethoxy]acetate (Compound 18.03) (120 mg, 64%). LCMS-ESI (pos.) m/z: 558.4 (M+1)⁺

### Step-4: Synthesis of 2-[4-[2-(carboxymethoxy)ethyl]piperazin-1-yl]ethyl-[(1S,3S)-3-[[5-(1-ethylpropyl)pyrazolo[1,5-a]pyrimidin-7-yl]amino]cyclopentyl]ammonium 2,2,2-trifluoroacetate (Compound 18.04)

To a stirred solution of tert-butyl 2-[2-[4-[2-[[(1S,3S)-3-[[5-(1-ethylpropyl)pyrazolo[1,5-a]pyrimidin-7-yl]amino]cyclopentyl]amino]ethyl]piperazin-1-yl]ethoxy]acetate (Compound 18.03) (50 mg, 0.09 mmol) in DCM (2 mL) at 0° C. was added trifluoroacetic acid (0.21 mL, 1.51 mmol). The reaction mixture was stirred at 0° C. for 3 h. After completion of the reaction as monitored by TLC, the reaction mixture was evaporated to dryness in vacuo, washed with n-pentane, and dried to give 2-[4-[2-(carboxymethoxy)ethyl]piperazin-1-yl]ethyl-[(1S,3S)-3-[[5-(1-ethylpropyl)pyrazolo[1,5-a]pyrimidin-7-yl]amino]cyclopentyl]ammonium 2,2,2-trifluoroacetate (Compound 18.04) (44 mg, 80%). LCMS-ESI (pos.) m/z: 502.0 (M+1)⁺

### Step-5: Synthesis of (2S,4R)-1-[(2S)-2-[[2-[2-[4-[2-[[(1S,3S)-3-[[5-(1-ethylpropyl)pyrazolo[1,5-a]pyrimidin-7-yl]amino]cyclopentyl]amino]ethyl]piperazin-1-yl]ethoxy]acetyl]amino]-3,3-dimethyl-butanoyl]-4-hydroxy-N-[[4-(4-methylthiazol-5-yl)phenyl]methyl]pyrrolidine-2-carboxamide formic acid salt (Compound 18.0)

To a stirred solution of 2-[4-[2-(carboxymethoxy)ethyl]piperazin-1-yl]ethyl-[(1S,3S)-3-[[5-(1-ethylpropyl)pyrazolo[1,5-a]pyrimidin-7-yl]amino]cyclopentyl]ammonium 2,2,2-trifluoroacetate (40 mg, 0.08 mmol) (Compound 18.04) in DMF (3 mL) at 0° C. were added [(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]ammonium chloride (55.86 mg, 0.12 mmol) and HATU (60.64 mg, 0.16 mmol). N,N-diisopropylethylamine (52.71 uL, 0.32 mmol) was then added to the reaction mixture. The reaction mixture was stirred at ambient temperature for 16 h. After completion of the reaction as monitored by LCMS, the reaction mixture was evaporated to dryness in vacuo. The crude residue was purified by Prep HPLC followed by lyophilization from MeCN/water to give (2S,4R)-1-[(2S)-2-[[2-[2-[4-[2-[[(1S,3S)-3-[[5-(1-ethylpropyl)pyrazolo[1,5-a]pyrimidin-7-yl]amino]cyclopentyl]amino]ethyl]piperazin-1-yl]ethoxy]acetyl]amino]-3,3-dimethyl-butanoyl]-4-hydroxy-N-[[4-(4-methylthiazol-5-yl)phenyl]methyl]pyrrolidine-2-carboxamide formic acid salt (Compound 18.0) (8 mg, 11%). LCMS-ESI (pos.) m/z: 914.6 (M+1)⁺

¹H NMR (400 MHz, DMSO-d6) δ 8.98 (s, 1H), 8.61 (t, J=6.0 Hz, 1H), 8.21 (s, 1H), 8.01 (d, J=2.2 Hz, 1H), 7.61 (d, J=7.6 Hz, 1H), 7.51-7.24 (m, 4H), 6.32 (d, J=2.3 Hz, 1H), 5.98 (s, 1H), 4.55 (d, J=9.4 Hz, 1H), 4.49-4.31 (m, 3H), 4.30-4.15 (m, 3H), 3.95 (s, 2H), 3.71-3.46 (m, 7H), 2.81 (d, J=7.3 Hz, 2H), 2.42 (s, 12H), 2.26-2.13 (m, 2H), 2.12-1.92 (m, 4H), 1.93-1.85 (m, 1H), 1.83-1.41 (m, 8H), 0.94 (s, 9H), 0.76 (t, J=7.3 Hz, 6H).

### Compound 19.0 2-(2,6-dioxo-3-piperidyl)-4-[2-[4-[2-[[(1S,3S)-3-[[5-(1-ethylpropyl)pyrazolo[1,5-a]pyrimidin-7-yl]amino]cyclopentyl]amino]ethyl]piperazin-1-yl]ethylamino]isoindoline-1,3-dione

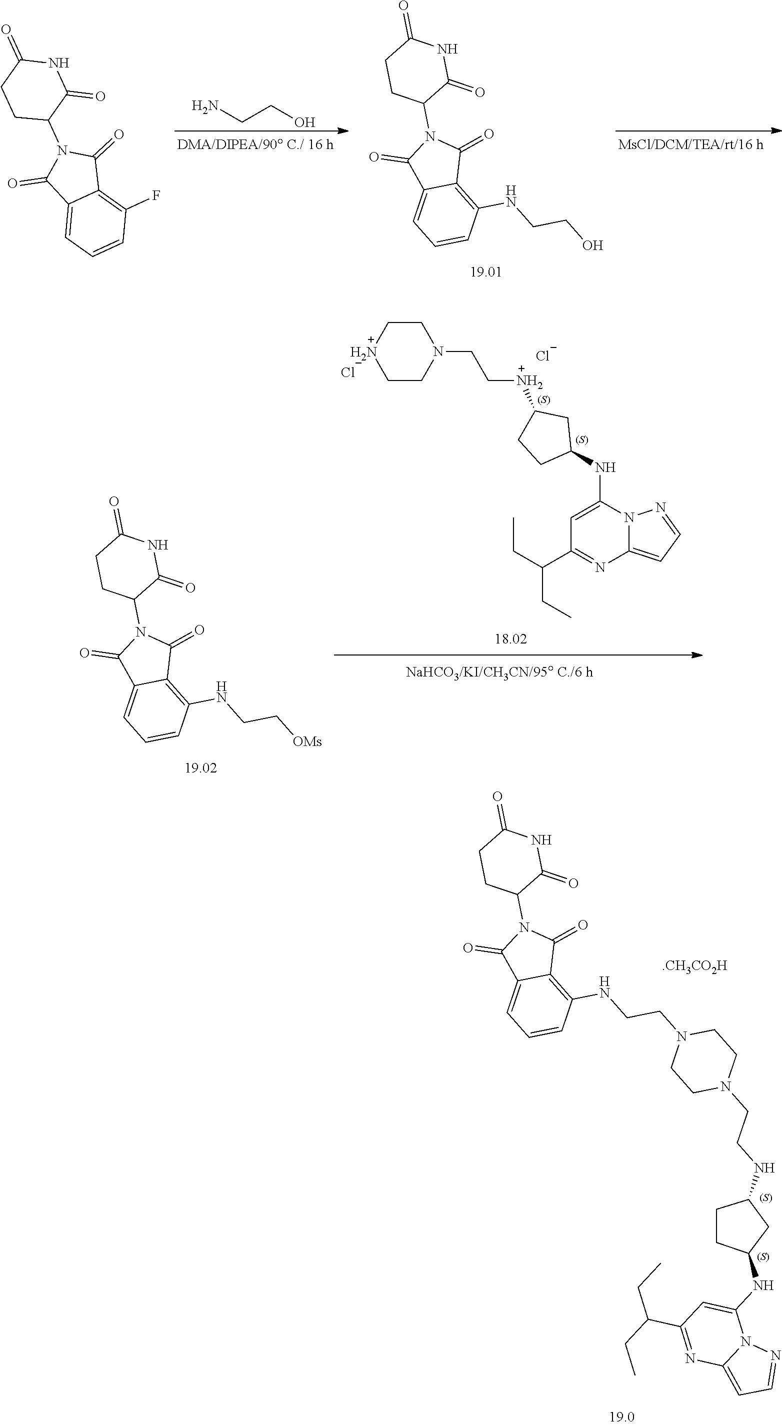

### Step-1: Synthesis of 2-(2,6-dioxo-3-piperidyl)-4-(2-hydroxyethylamino)isoindoline-1,3-dione (Compound 19.01)

2-(2,6-dioxo-3-piperidyl)-4-fluoro-isoindoline-1,3-dione (1 g, 3.62 mmol) (Compound 5.01) and 2-aminoethanol (331.69 mg, 5.43 mmol) were taken in DMA (10 mL), followed by the addition of DIPEA (1.95 mL, 10.86 mmol). The reaction mixture was heated at 90° C. for 16 h. The reaction mixture was then taken up in EtOAc (20 mL), and the organic layer was washed with water (2×20 ml) followed by saturated brine solution (1×20 mL). The organic layer was separated, dried over anhydrous Na₂SO₄ and concentrated to dryness in vacuo. The crude residue was purified by Combiflash column chromatography (silica gel), eluent 5% MeOH in DCM. The desired fractions were concentrated to dryness in vacuo to give 2-(2,6-dioxo-3-piperidyl)-4-(2-hydroxyethylamino)isoindoline-1,3-dione (Compound 19.01) (200 mg, 17%). LCMS-ESI (pos.) m/z: 318.0 (M+1)⁺

### Step-2: Synthesis of 2-(2,6-dioxo-3-piperidyl)-4-(2-hydroxyethylamino)isoindoline-1,3-dione (Compound 19.02)

2-(2,6-dioxo-3-piperidyl)-4-(2-hydroxyethylamino)isoindoline-1,3-dione (Compound 19.01) (80 mg, 0.25 mmol) was taken in DCM (5 mL) and cooled to 0° C. Methanesulphonyl chloride (97.57 uL, 0.76 mmol) and Et₃N (176.84 uL, 1.26 mmol) were then added. The reaction mixture was stirred vigorously for 16 h at ambient temperature. The reaction mixture was concentrated to dryness in vacuo, and the crude was purified by Combiflash column chromatography (silica gel), eluent 10% MeOH in DCM. The desired fractions were concentrated to dryness in vacuo to give 2-[[2-(2,6-dioxo-3-piperidyl)-1,3-dioxo-isoindolin-4-yl]amino]ethyl methanesulfonate (Compound 19.02) (40 mg, 40%). LCMS-ESI (pos.) m/z: 396.1 (M+1)⁺

### Step-3: Synthesis of acetic acid 2-(2,6-dioxo-3-piperidyl)-4-[2-[4-[2-[[(1S,3S)-3-[[5-(1-ethylpropyl)pyrazolo[1,5-a]pyrimidin-7-yl]amino]cyclopentyl]amino]ethyl]piperazin-1-yl]ethylamino]isoindoline-1,3-dione (Compound 19.0)

To a stirred solution of 2-piperazin-4-ium-1-ylethyl-[(1S,3S)-3-[[5-(1-ethylpropyl)pyrazolo[1,5-a]pyrimidin-7-yl]amino]cyclopentyl]ammonium dichloride (Compound 18.02) (23.9 mg, 0.05 mmol) in dry MeCN (5 mL) were added sodium bicarbonate (42.5 mg, 0.51 mmol) and 2-[[2-(2,6-dioxo-3-piperidyl)-1,3-dioxo-isoindolin-4-yl]amino]ethyl methanesulfonate (Compound 19.03) (20 mg, 0.05 mmol) and stirred at room temperature. Then KI (839.68 ug, 0.01 mmol) added and the reaction mixture was stirred at 95° C. for 16 h. After completion of the reaction, solvent was removed in vacuo. The crude reaction mixture was purified by Prep HPLC followed by lyophilization from MeCN/water to give acetic acid 2-(2,6-dioxo-3-piperidyl)-4-[2-[4-[2-[[(1S,3S)-3-[[5-(1-ethylpropyl)pyrazolo[1,5-a]pyrimidin-7-yl]amino]cyclopentyl]amino]ethyl]piperazin-1-yl]ethylamino]isoindoline-1,3-dione (Compound 19.0) (4.22 mg, 11%). LCMS-ESI (pos.) m/z: 699.4 (M+1)⁺

¹H NMR (400 MHz, Chloroform-d) δ 7.93 (d, J=2.3 Hz, 1H), 7.49 (t, J=7.8 Hz, 1H), 7.09 (d, J=7.1 Hz, 1H), 6.86 (d, J=8.5 Hz, 1H), 6.68 (s, 1H), 6.42 (d, J=2.4 Hz, 1H), 6.18 (s, 1H), 5.78 (s, 1H), 4.91 (dd, J=5.2, 12.1 Hz, 1H), 1.32-1.15 (m, 5H), 4.19 (d, J=6.9 Hz, 1H), 3.43-3.26 (m, 3H), 2.95-2.62 (m, 6H), 2.61-2.42 (m, 7H), 2.42-2.32 (m, 1H), 2.20-2.07 (m, 2H), 2.09-1.93 (m, 4H), 1.77-1.60 (m, 6H), 1.64-1.49 (m, 2H), 1.24 (s, 3H), 0.84 (t, J=7.3 Hz, 6H).

### Compound 20.0 N-[3-[1-[2-[2-[[2-(2,6-dioxo-3-piperidyl)-1,3-dioxo-isoindolin-4-yl]amino]ethoxy]ethyl]triazol-4-yl]propyl]-2-[[(1S,3S)-3-[[5-(1-ethylpropyl)pyrazolo[1,5-a]pyrimidin-7-yl]amino]cyclopentyl]amino]acetamide

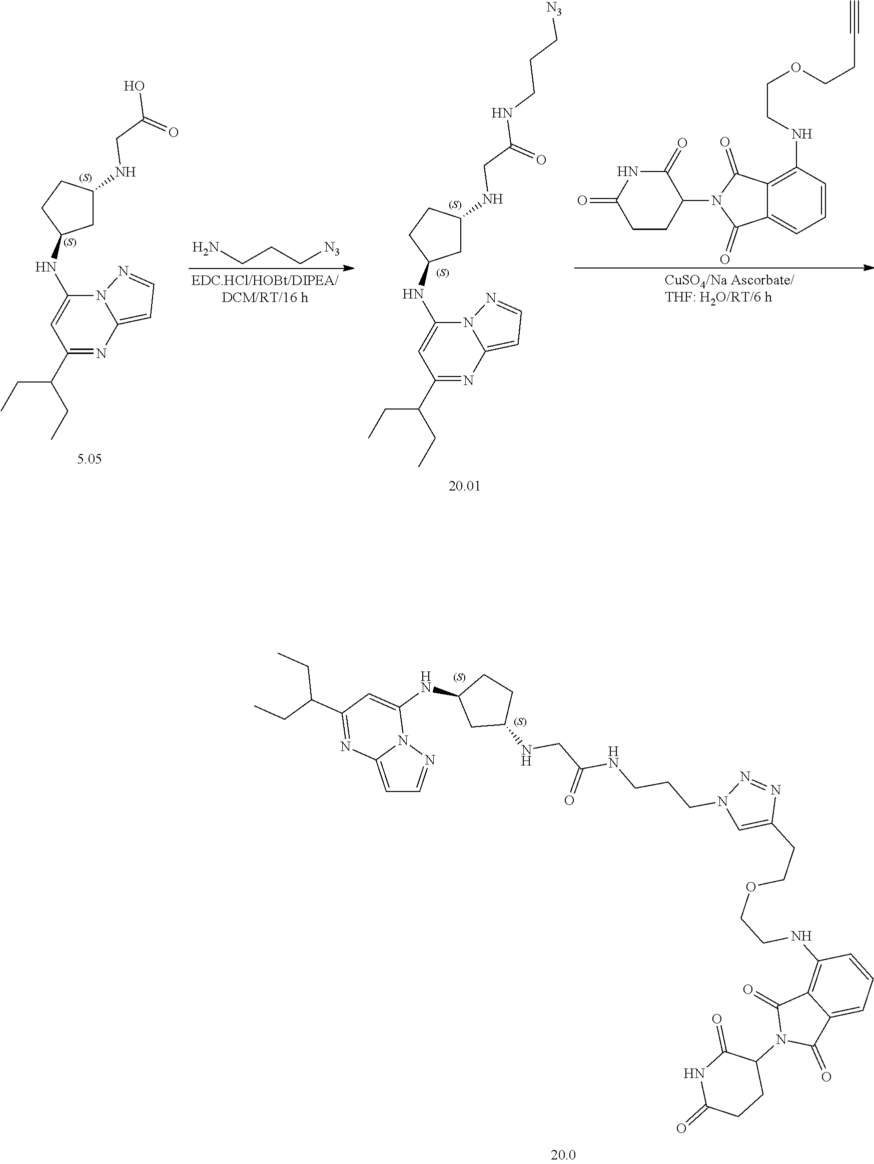

### Step-1: Synthesis of N-(3-azidopropyl)-2-[[(1S,3S)-3-[[5-(1-ethylpropyl)pyrazolo[1,5-a]pyrimidin-7-yl]amino]cyclopentyl]amino]acetamide (Compound 20.01)

To a solution of 2-[[(1S,3S)-3-[[5-(1-ethylpropyl)pyrazolo[1,5-a]pyrimidin-7-yl]amino]cyclopentyl]amino]acetic acid 2,2,2-trifluoroacetic acid (310 mg, 0.90 mmol) and 3-azidopropan-1-amine (0.09 mL, 0.90 mmol) in DMF (5 mL) at 0° C. were added DIPEA (0.47 mL, 2.69 mmol), EDC·HCl (258.05 mg, 1.35 mmol), and HOBt (181.72 mg, 1.35 mmol), and stirred at RT for 16 h. The reaction mixture was extracted with EtOAc, then washed with water, followed by saturated brine solution. The organic layer was dried over anhydrous Na₂SO₄ and concentrated in vacuo. The crude was purified by Combiflash column chromatography (silica gel), eluent 5% MeOH in DCM to give N-(3-azidopropyl)-2-[[(1S,3S)-3-[[5-(1-ethylpropyl)pyrazolo[1,5-a]pyrimidin-7-yl]amino]cyclopentyl]amino]acetamide (Compound 20.01) (210 mg, 47%). LCMS-ESI (pos.) m/z: 428.1 (M+1)⁺

### Step-2: Synthesis of N-[3-[1-[2-[2-[[2-(2,6-dioxo-3-piperidyl)-1,3-dioxo-isoindolin-4-yl]amino]ethoxy]ethyl]triazol-4-yl]propyl]-2-[[(1S,3S)-3-[[5-(1-ethylpropyl)pyrazolo[1,5-a]pyrimidin-7-yl]amino]cyclopentyl]amino]acetamide (Compound 20.0)

To a solution of N-(3-azidopropyl)-2-[[(1S,3S)-3-[[5-(1-ethylpropyl)pyrazolo[1,5-a]pyrimidin-7-yl]amino]cyclopentyl]amino]acetamide (Compound 20.01) (34.73 mg, 0.08 mmol) in THF (2 mL) were added 4-(2-but-3-ynoxyethylamino)-2-(2,6-dioxo-3-piperidyl)isoindoline-1,3-dione (30 mg, 0.08 mmol), CuSO₄ (2.59 mg, 0.02 mmol), sodium ascorbate (3.22 mg, 0.02 mmol), and water (0.2 mL), and stirred at ambient temperature for 16 h. The reaction mixture was evaporated in vacuo, and the crude residue was purified by Prep-HPLC followed by lyophilization from MeCN:H₂O to give N-[3-[1-[2-[2-[[2-(2,6-dioxo-3-piperidyl)-1,3-dioxo-isoindolin-4-yl]amino]ethoxy]ethyl]triazol-4-yl]propyl]-2-[[(1S,3S)-3-[[5-(1-ethylpropyl)pyrazolo[1,5-a]pyrimidin-7-yl]amino]cyclopentyl]amino]acetamide (Compound 20.0) (10 mg, 15%). LCMS-ESI (pos.) m/z: 797.5 (M+1)⁺

¹H NMR (400 MHz, DMSO-d₆) δ 12.83-9.09 (m, 1H), 8.19 (s, 1H), 7.95 (s, 1H), 7.76 (s, 1H), 7.63 (s, 1H), 7.57 (t, J=7.9 Hz, 1H), 7.12 (d, J=8.6 Hz, 1H), 7.04 (d, J=7.1 Hz, 1H), 6.96 (d, J=7.4 Hz, 1H), 6.52 (t, J=5.8 Hz, 1H), 6.29 (s, 1H), 5.96 (s, 1H), 5.01 (dd, J=5.5, 12.7 Hz, 1H), 4.31 (t, J=7.0 Hz, 2H), 4.23 (q, J=7.1 Hz, 1H), 3.73 (t, J=6.8 Hz, 2H), 3.66 (t, J=5.5 Hz, 2H), 3.49 (q, J=5.6 Hz, 2H), 3.27 (q, J=6.0 Hz, 1H), 3.25-3.09 (m, 5H), 2.94-2.81 (m, 4H), 2.68-2.53 (m, 2H), 2.32-2.19 (m, 1H), 2.13-1.85 (m, 5H), 1.80-1.57 (m, 5H), 1.52-1.38 (m, 1H), 0.82 (t, J=7.4 Hz, 6H).

### Compound 21.0 2-(2,6-dioxo-3-piperidyl)-4-[3-[4-[2-[[(1S,3S)-3-[[5-[(E)-1-ethylprop-1-enyl]pyrazolo[1,5-a]pyrimidin-7-yl]amino]cyclopentyl]amino]acetyl]piperazin-1-yl]propylamino]isoindoline-1,3-dione

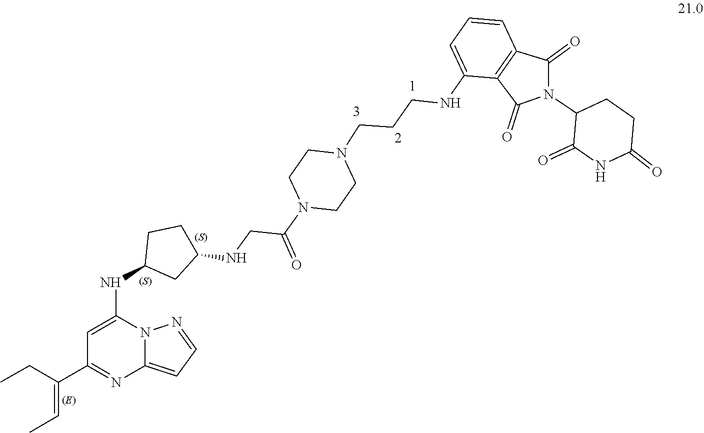

### Step 1. Synthesis of ethyl 7-chloropyrazolo[1,5-a]pyrimidine-5-carboxylate (Compound 21.01)

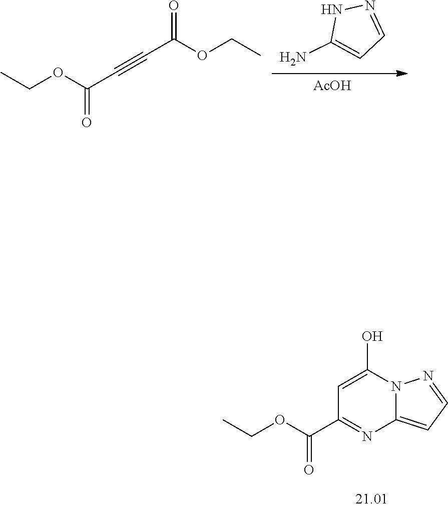

To a solution of diethyl but-2-ynedioate (50 g, 293.84 mmol) in acetic acid (300 mL) was added 1H-pyrazol-5-amine (24.42 g, 293.84 mmol), the mixture was stirred at 110° C. for 8 h. The reaction was cooled to room temperature and the mixture was filtered. The filter cake was washed with acetic acid (20 mL*2), followed by methyl tert-butyl ether (30 mL*5), then concentrated in vacuum to give a crude product. Compound ethyl 7-hydroxypyrazolo[1,5-a]pyrimidine-5-carboxylate (Compound 21.01) (42 g, 69%) was obtained as a pale brown solid.

¹H NMR (400 MHz, DMSO-d6) δ 12.87 (s, 1H), 7.95 (s, 1H), 6.28 (s, 2H), 4.40 (q, J=7.2 Hz, 2H), 1.36 (t, J=7.2 Hz, 3H)

### Step 2. Synthesis of ethyl 7-chloropyrazolo[1,5-a]pyrimidine-5-carboxylate (Compound 21.02)

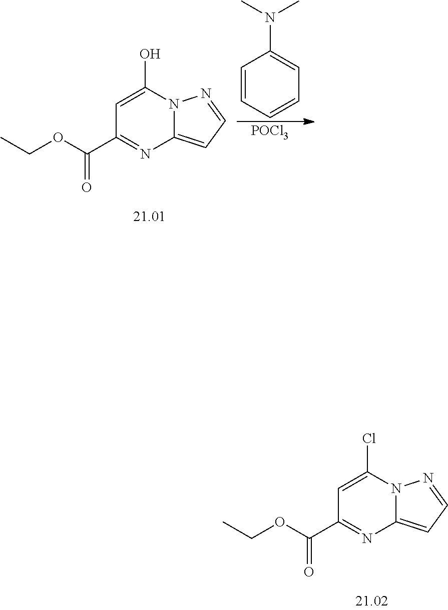

To a solution of ethyl 7-hydroxypyrazolo[1,5-a]pyrimidine-5-carboxylate (Compound 21.01) (20 g, 96.53 mmol) in POCl₃ (200 mL, 2139.6 mmol, 32.8 g) was added N,N-dimethylaniline (8.19 g, 67.57 mmol) dropwise slowly. The mixture was stirred at 110° C. for 8 h. LCMS showed a small amount of starting material remained and desired mass was detected. POCl₃ was removed in vacuo. The residue was dissolved in DCM (10 mL), poured into ice water (50 mL), extracted with DCM (100 mL*3). The combined organic layers were dried over anhydrous sodium sulfate and concentrated to afford the crude product. The crude product was purified by CombiFlash column (ISCO 120 g silica, 0-30% ethyl acetate in petroleum ether, 60 min gradient). Compound ethyl 7-chloropyrazolo[1,5-a]pyrimidine-5-carboxylate (Compound 21.02) (18.5 g, 85%) was obtained as yellow solid. LCMS (pos.) m/z 226.2, [M+1]⁺

¹H NMR (400 MHz, DMSO-d6) δ=8.51 (d, J=2.4 Hz, 1H), 7.82 (s, 1H), 7.19 (d, J=2.5 Hz, 1H), 4.40 (q, J=7.1 Hz, 2H), 1.36 (t, J=7.1 Hz, 3H)

### Step 3. Synthesis of ethyl 7-[[(1S,3S)-3-(tert-butoxycarbonylamino)cyclopentyl]amino]pyrazolo[1,5-a]pyrimidine-5-carboxylate (Compound 21.03)

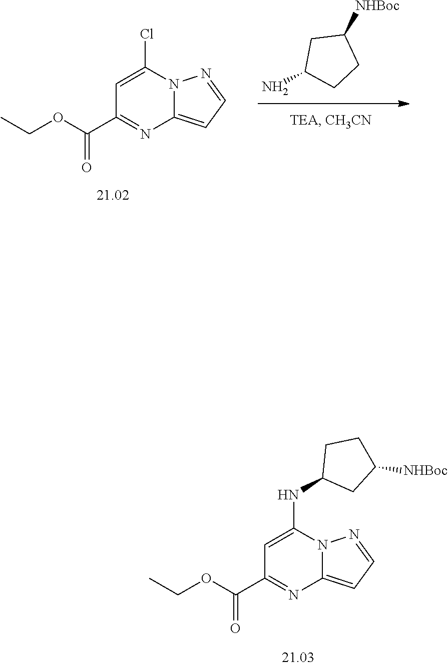 [SMILES: CCOC(=O)c1cc(Cl)n2nccc2n1 ; CC(C)(C)OC(=O)N[C@H]1CC[C@H](N)C1 ; CCOC(=O)c1cc(N[C@H]2CC[C@H](NC(=O)OC(C)(C)C)C2)n2nccc2n1]

To a stirred solution of ethyl 7-chloropyrazolo[1,5-a]pyrimidine-5-carboxylate (Compound 21.02) (8 g, 35.46 mmol) in MeCN (80 mL) was added TEA (4.97 mL, 35.46 mmol) and tert-Butyl ((1S,3S)-3-aminocyclopentyl)carbamate (7.1 g, 35.46 mmol). Then the mixture was stirred at 85° C. for 2 h. LC-MS showed starting material was consumed completely and desired mass was detected. TLC (petroleum ether:ethyl acetate=1:1, Rf=0.5, ultraviolet) indicated reactant was consumed completely and one new spot formed. The mixture was concentrated to afford the crude product. The crude product was purified by Combiflash column (ISCO 12 g silica gel, 0-50% ethyl acetate in petroleum ether, 50 min gradient). Compound ethyl 7-[[(1S,3S)-3-(tert-butoxycarbonylamino)cyclopentyl]amino]pyrazolo[1,5-a]pyrimidine-5-carboxylate (Compound 21.03) (11.6 g, 82%) was obtained as yellow solid.

ee %=100%. LCMS (pos.) m/z 390.3, [M+1]⁺

¹H NMR (400 MHz, DMSO-d6) δ=8.29-8.14 (m, 2H), 7.02 (br d, J=6.9 Hz, 1H), 6.71 (s, 1H), 6.65 (d, J=2.3 Hz, 1H), 4.39-4.25 (m, 3H), 4.00 (br d, J=5.3 Hz, 1H), 2.21-2.08 (m, 1H), 2.07-1.95 (m, 2H), 1.94-1.85 (m, 1H), 1.80-1.67 (m, 1H), 1.56-1.43 (m, 1H), 1.39 (s, 9H), 1.34 (t, J=7.1 Hz, 3H.

### Step 4. Synthesis of tert-butyl N-[(1S,3S)-3-[[5-(1-ethyl-1-hydroxy-propyl)pyrazolo[1,5-a]pyrimidin-7-yl]amino]cyclopentyl]carbamate (Compound 21.04)

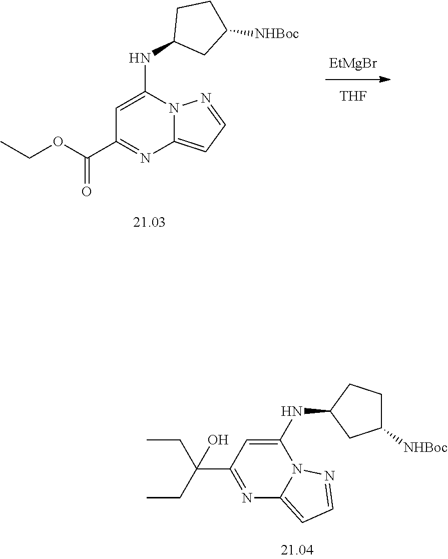 [SMILES: "EtMgBr" ; CCOC(=O)c1cc(N[C@H]2CC[C@H](NC(=O)OC(C)(C)C)C2)n2nccc2n1 ; CCC(O)(CC)c1cc(N[C@H]2CC[C@H](NC(=O)OC(C)(C)C)C2)n2nccc2n1 ; "THF"]

To a stirred solution of ethyl 7-[[(1S,3S)-3-(tert-butoxycarbonylamino)cyclopentyl]amino]pyrazolo[1,5-a]pyrimidine-5-carboxylate (Compound 21.03) (12 g, 30.81 mmol) in THF (120 mL) was added bromo(ethyl)magnesium (41.08 mL, 123.25 mmol) (3M in Et₂O) at 0° C. Then the mixture was stirred at 20° C. for 16 h. LC-MS showed starting material was consumed completely and desired mass was detected. TLC (petroleum ether:ethyl acetate=1:1, Rf=0.5, ultraviolet) indicated reactant was consumed completely and two main new spots formed. The mixture was quenched with saturated NH4Cl (30 mL) at 0° C., stirred for another 10 min, extracted with ethyl acetate (50 mL*3). The combined organic layers were dried over anhydrous sodium sulfate and concentrated to afford the crude product. The crude product was purified by Combiflash column (ISCO 80 g silica gel, 0-30% ethyl acetate in petroleum ether, gradient over 50 min). Compound tert-butyl N-[(1S,3S)-3-[[5-(1-ethyl-1-hydroxy-propyl)pyrazolo[1,5-a]pyrimidin-7-yl]amino]cyclopentyl]carbamate (4.3 g, 10.341 mmol, 33.56%) was obtained as yellow gum and tert-butyl N-[(1S,3S)-3-[(5-propanoylpyrazolo[1,5-a]pyrimidin-7-yl)amino]cyclopentyl]carbamate (Compound 21.04) (lg, 8%) was obtained as a pale yellow solid. ee %=100%. LCMS (pos.) m/z 404.4, [M+1]⁺

### Step 5. Synthesis of (1S,3S)—N3-[5-[(E)-1-ethylprop-1-enyl]pyrazolo[1,5-a]pyrimidin-7-yl]cyclopentane-1,3-diamine (Compound 21.05)

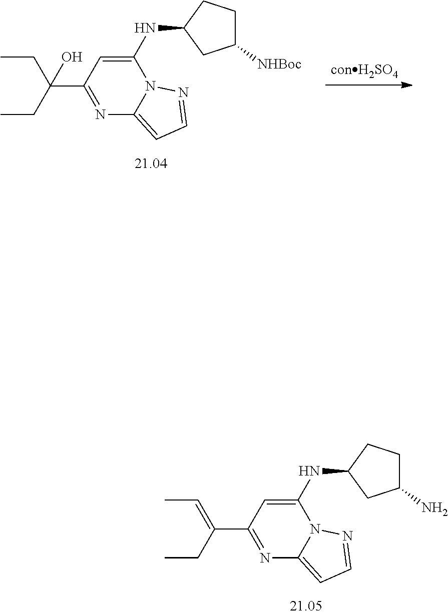 [SMILES: CCC(O)(CC)c1cc(N[C@H]2CC[C@H](NC(=O)OC(C)(C)C)C2)n2nccc2n1 ; C/C=C(\CC)c1cc(N[C@H]2CC[C@H](N)C2)n2nccc2n1]

A solution of tert-butyl N-[(1S,3S)-3-[[5-(1-ethyl-1-hydroxy-propyl)pyrazolo[1,5-a]pyrimidin-7-yl]amino]cyclopentyl]carbamate (1 g, 2.48 mmol) in H₂SO₄ (6 mL, 2.48 mmol) was stirred at 110° C. for 1 h. LCMS and HPLC showed reactant was consumed completely and desired MS was detected. The mixture was poured into ice water (10 ML), adjusted to pH=8-9 by adding ammonia, and extracted with CHCl₃/iPrOH=3/1 (10 mL*5). The combined organic layers were dried over anhydrous sodium sulfate and concentrated to afford the crude product. The crude residue was purified by prep-HPLC (Kromasil C18 250*50 mm*10 um column; 10-45% acetonitrile in 0.05% ammonia and 10 mM ammonium bicarbonate solution in water, 10 min gradient). Compound (1S,3S)—N3-[5-[(Z)-1-ethylprop-1-enyl]pyrazolo[1,5-a]pyrimidin-7-yl]cyclopentane-1,3-diamine (30 mg, 4%) was obtained as a yellow oil and (1S,3S)—N3-[5-[(E)-1-ethylprop-1-enyl]pyrazolo[1,5-a]pyrimidin-7-yl]cyclopentane-1,3-diamine (Compound 21.05) (350 mg, 1.2 mmol, 48%) was obtained as a yellow gum.

ee %=94.62%. LCMS (pos.) m/z 286.2, [M+1]⁺

¹H NMR (400 MHz, MeOD-d₄) δ=7.95 (d, J=2.4 Hz, 1H), 6.46 (d, J=2.4 Hz, 1H), 6.46 (t, J=7.2 Hz, 1H), 6.21 (d, J=26.4 Hz, 1H), 4.36 (q, J=6.8 Hz, 1H), 3.57 (t, J=2.4 Hz, 1H), 3.32 (d, J=15.6 Hz, 1H), 2.69 (q, J=3.6 Hz, 2H), 2.38 (d, J=4 Hz, 1H), 2.01 (m, J=3.6 Hz, 2H), 1.89-1.96 (m, 3H), 1.76-1.87 (m, 1H), 1.45-1.51 (m, 1H), 1.02 (d, J=3.6 Hz, 3H)

### Step 6. Synthesis of tert-butyl 2-[[(1S,3S)-3-[[5-[(E)-1-ethylprop-1-enyl]pyrazolo[1,5-a]pyrimidin-7-yl]amino]cyclopentyl]amino]acetate (Compound 21.06)

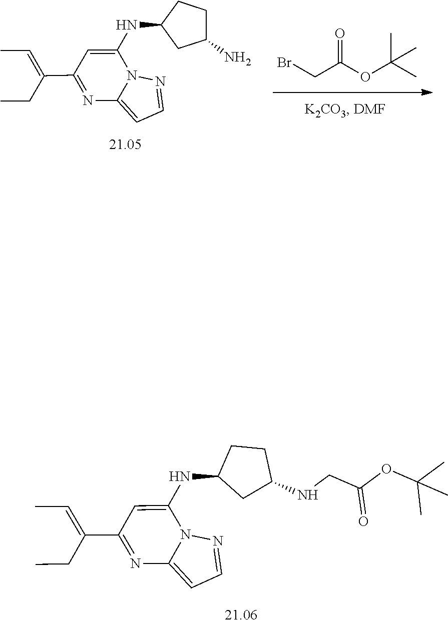

To a solution of (1S,3S)—N3-[5-[(E)-1-ethylprop-1-enyl]pyrazolo[1,5-a]pyrimidin-7-yl]cyclopentane-1,3-diamine (Compound 21.05) (390 mg, 1.37 mmol) in DMF (4 mL) was added K₂CO₃ (377.17 mg, 2.73 mmol) at 0° C., and the mixture was stirred at 0° C. for 0.5 h. Then tert-butyl 2-bromoacetate (266.55 mg, 1.37 mmol) was added at 0° C., the resulting mixture was stirred at 0° C. for 2 h. LCMS showed a small amount of reactant was remained and one main peak with desired MS was detected. TLC (petroleum ether:ethyl acetate=0:1, R_{f}=0.45, ultraviolet) indicated a small amount of reactant was remained and one new spot formed. The reaction mixture was poured to water (6 mL), and the resulting mixture was extracted with ethyl acetate (10 mL*3). The combined organic layers were washed with water (5 mL), brine (5 mL), dried over anhydrous sodium sulfate, and concentrated in vacuum to give a crude product. The crude product was purified by Combiflash column (ISCO 20 g silica gel, 0-35% ethyl acetate in petroleum ether, gradient over 20 min). Compound tert-butyl 2-[[(1S,3S)-3-[[5-[(E)-1-ethylprop-1-enyl]pyrazolo[1,5-a]pyrimidin-7-yl]amino]cyclopentyl]amino]acetate (Compound 21.06) (260 mg, 44%) was obtained as a pale yellow gum. ee=100%. LCMS (pos.) m/z 400.2, [M+1]⁺

¹H NMR (400 MHz, MeOD-d₄) δ=7.97 (s, 1H), 6.35 (d, J=2.4 Hz, 1H), 6.26-6.35 (m, 1H), 6.21 (s, 1H), 4.88-4.86 (m, 1H), 4.59-4.57 (m, 1H) 4.28-4.35 (m, 1H), 3.31-3.38 (m, 1H), 2.99 (s, 3H), 2.64-2.86 (m, 2H), 2.21-2.34 (m, 1H), 2.01-2.19 (m, 1H), 1.96-2.01 (m, 2H), 1.88 (d, J=3.2 Hz, 3H), 1.87-1.60 (m, 1H), 1.53-1.54 (m, 1H), 1.47 (s, 9H), 1.02 (t, J=3.2 Hz, 3H)

### Step 7. Synthesis of 2-[[(1S,3S)-3-[[5-[(E)-1-ethylprop-1-enyl]pyrazolo[1,5-a]pyrimidin-7-yl]amino]cyclopentyl]amino]acetic acid dihydrochloride (Compound 21.07)

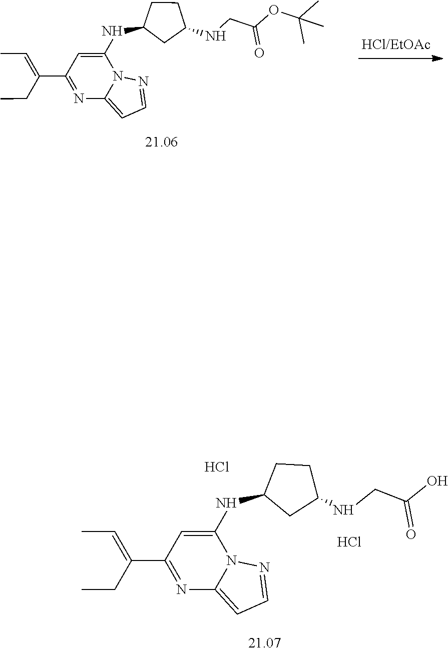

A solution of tert-butyl 2-[[(1S,3S)-3-[[5-[(E)-1-ethylprop-1-enyl]pyrazolo[1,5-a]pyrimidin-7-yl]amino]cyclopentyl]amino]acetate (Compound 21.06) (250 mg, 0.6300 mmol) in HCl (5 mL, 20 mmol) (4M in EtOAc) was stirred at 25° C. for 2 h. LCMS showed a small amount of reactant was remained and one main peak with desired MS was detected. Compound 2-[[(1S,3S)-3-[[5-[(E)-1-ethylprop-1-enyl]pyrazolo[1,5-a]pyrimidin-7-yl]amino]cyclopentyl]amino]acetic acid; dihydrochloride (Compound 21.07) (210 mg, 75%) was obtained as a pale yellow solid. ee=100%. LCMS (pos.) m/z 344.1, [M+1]⁺

¹H NMR (400 MHz, MeOD-d₄) δ=8.21 (d, J=2.0 Hz, 1H), 6.61 (s, 1H), 6.51-6.55 (m, 1H), 6.47-6.49 (m, 1H), 4.84 (s, 1H), 3.98 (s, 2H), 3.91-3.95 (m, 1H), 2.75-2.70 (m, 2H), 2.43-2.48 (m, 3H), 2.35-2.43 (m, 1H), 1.98 (d, J=6.8 Hz, 3H), 1.94-1.95 (m, 2H), 1.09 (t, J=4.4 Hz, 3H)

### Step 8. Synthesis of tert-butyl 4-[3-[[2-(2,6-dioxo-3-piperidyl)-1,3-dioxo-isoindolin-4-yl]amino]propyl]piperazine-1-carboxylate (Compound 21.08)

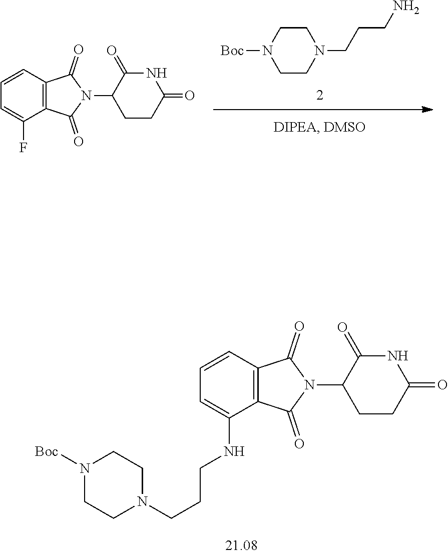 [SMILES: O=C1CCC(N2C(=O)c3cccc(F)c3C2=O)C(=O)N1 ; CC(C)(C)OC(=O)N1CCN(CCCN)CC1 ; CC(C)(C)OC(=O)N1CCN(CCCNc2cccc3c2C(=O)N(C2CCC(=O)NC2=O)C3=O)CC1]

To a stirred solution of 2-(2,6-dioxo-3-piperidyl)-4-fluoro-isoindoline-1,3-dione (50 mg, 0.1800 mmol), tert-butyl 4-(3-aminopropyl)piperazine-1-carboxylate (44.05 mg, 0.1800 mmol) in DMSO (0.5000 mL) was added DIPEA (0.06 mL, 0.3600 mmol), and stirred at 80° C. for 4 h. LC-MS showed reactant was consumed completely and main desired mass was detected. The mixture was poured into water (4 mL) and extracted with ethyl acetate (3*5 mL). The organic layer was washed with brine (4 mL), dried over anhydrous Na₂SO₄, then filtered and concentrated in vacuo. The crude product was purified by Prep-TLC (Dichloromethane:methanol=10/1, R_{f}=0.3) to give tert-butyl 4-[3-[[2-(2,6-dioxo-3-piperidyl)-1,3-dioxo-isoindolin-4-yl]amino]propyl]piperazine-1-carboxylate (Compound 21.08) (50 mg, 55%). LCMS (pos.) m/z 486.2 [M+1]⁺

### Step 9. Synthesis of 2-(2,6-dioxo-3-piperidyl)-4-(3-piperazin-1-ylpropylamino)isoindoline-1,3-dione (Compound 21.09)

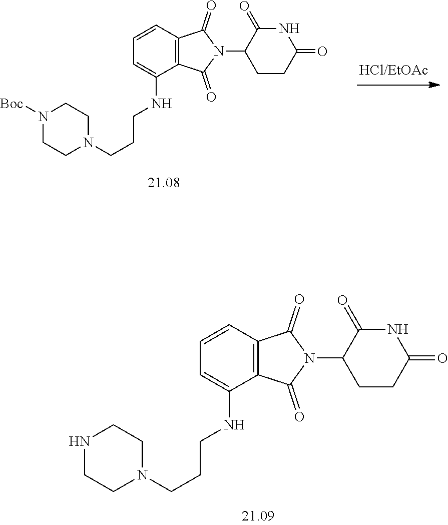 [SMILES: CC(C)(C)OC(=O)N1CCN(CCCNc2cccc3c2C(=O)N(C2CCC(=O)NC2=O)C3=O)CC1 ; O=C1CCC(N2C(=O)c3cccc(NCCCN4CCNCC4)c3C2=O)C(=O)N1]

To a round bottom flask containing tert-butyl 4-[3-[[2-(2,6-dioxo-3-piperidyl)-1,3-dioxo-isoindolin-4-yl]amino]propyl]piperazine-1-carboxylate (Compound 21.08) (30 mg, 0.0600 mmol) was added HCl/EtOAc (2 mL, 8 mmol). The mixture was stirred at 20° C. for 2 h. TLC (DCM:MeOH=10:1, R_{f}=0.1) showed starting material was consumed completely and one new spot formed. The reaction mixture was concentrated under reduced pressure to give 2-(2,6-dioxo-3-piperidyl)-4-(3-piperazin-1-ylpropylamino)isoindoline-1,3-dione (Compound 21.09) (25 mg, 89%) as a yellow solid.

Step 10. Synthesis of 2-(2,6-dioxo-3-piperidyl)-4-[3-[4-[2-[[(1S,3S)-3-[[5-[(E)-1-ethylprop-1-enyl]pyrazolo[1,5-a]pyrimidin-7-yl]amino]cyclopentyl]amino]acetyl]piperazin-1-yl]propylamino]isoindoline-1,3-dione; 2,2,2-trifluoroacetic acid (Compound 21.0)

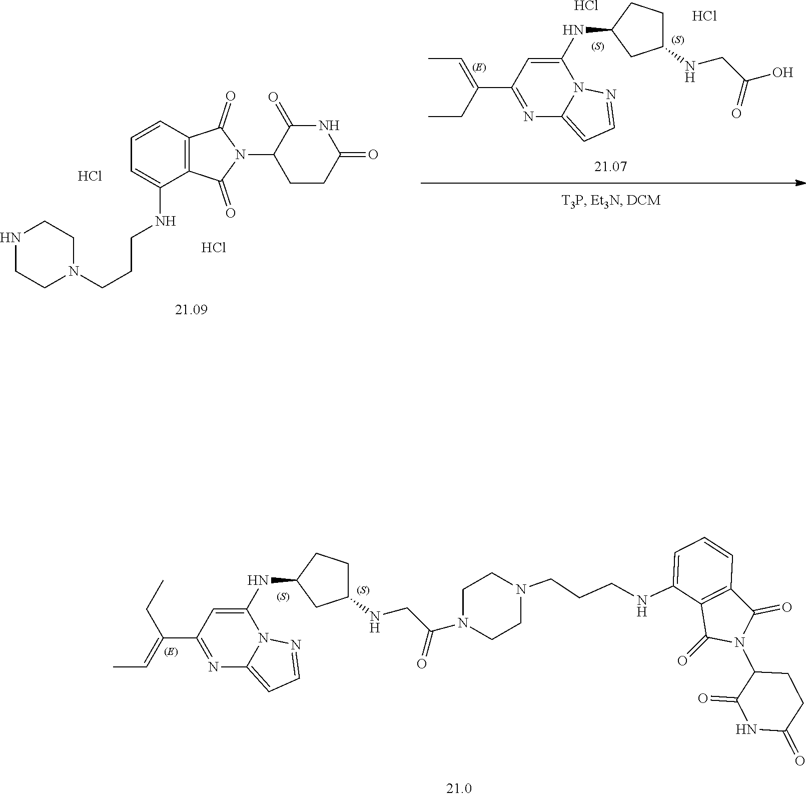

To a solution of 2-[[(1S,3S)-3-[[5-[(E)-1-ethylprop-1-enyl]pyrazolo[1,5-a]pyrimidin-7-yl]amino]cyclopentyl]amino]acetic acid; dihydrochloride (Compound 21.07) (15 mg, 0.0400 mmol), 2-(2,6-dioxo-3-piperidyl)-4-(3-piperazin-1-ylpropylamino)isoindoline-1,3-dione dihydrochloride (Compound 21.09) (20.42 mg, 0.0400 mmol), and TEA (0.03 mL, 0.1800 mmol) in DCM (1 mL) was added T₃P (45.85 mg, 0.0700 mmol), and the mixture was stirred at 25° C. for 0.5 h. LCMS showed that the reactant was consumed completely and one main peak with desired MS was detected. The reaction mixture was filtered, and the filtrate was purified by prep-HPLC (Phenomenex luna C18 100*40 mm*5 um column; 5-30% acetonitrile in an a 0.1% trifluoroacetic acid solution in water, 8 min gradient). Compound 2-(2,6-dioxo-3-piperidyl)-4-[3-[4-[2-[[(1S,3S)-3-[[5-[(E)-1-ethylprop-1-enyl]pyrazolo[1,5-a]pyrimidin-7-yl]amino]cyclopentyl]amino]acetyl]piperazin-1-yl]propylamino]isoindoline-1,3-dione; 2,2,2-trifluoroacetic acid (Compound 21.0) (10 mg, 28%) was obtained as a yellow solid. LCMS (pos.)m/z 363.2 [1/2M+1]⁺.

¹H NMR (400 MHz, MeOD-d₄) δ=8.19 (d, J=1.9 Hz, 1H), 7.61 (t, J=7.8 Hz, 1H), 7.17-7.09 (m, 2H), 6.54 (d, J=2.1 Hz, 1H), 6.48-6.41 (m, 2H), 5.08-5.06 (m, 1H), 4.68-4.66 (m, 1H), 4.23 (br s, 2H), 4.02-3.62 (m, 6H), 3.60-3.45 (m, 2H), 3.26-3.24 (m, 3H), 3.00-2.82 (m, 2H), 2.81-2.66 (m, 4H), 2.57-2.33 (m, 5H), 2.19-2.08 (m, 3H), 1.98 (br d, J=6.9 Hz, 5H), 1.74 (br d, J=6.9 Hz, 1H), 1.13-1.06 (m, 3H)

The compounds in Table 1 were prepared using the synthetic procedures as described above in Compound 1.0 to Compound 21.0 and corresponding reagents.

TABLE 1

| Compound | MF | Mass | [M + X] | Mass Found |
|---|---|---|---|---|
| 22.0 | C40H52N12O6 | 796.41 | [M + 1] | 797.3 |
| 23.0 | C40H51ClN12O6 | 830.37 | [M + 1] | 831.4 |
| 24.0 | C40H51ClN12O6 | 830.37 | [M + 1] | 831.3 |
| 25.0 | C47H65N13O6S | 939.49 | [M + 1] | 940.6 |
| 26.0 | C49H69N13O7S | 983.52 | [M + 1] | 984.6 |
| 27.0 | C51H73N13O8S | 1027.54 | [M + 1] | 1028.4 |
| 28.0 | C53H77N13O9S | 1071.57 | [M + 1] | 1072.5 |
| 29.0 | C54H69N13O7S | 1043.52 | [M + 1] | 1044.6 |
| 30.0 | C56H73N13O8S | 1087.54 | [M + 1] | 1088.3 |
| 31.0 | C58H77N13O9S | 1131.57 | [M + 1] | 1132.6 |
| 32.0 | C60H81N13O10S | 1175.60 | [M + 1] | 1176.5 |
| 33.0 | C45H62N12O5S | 882.47 | [M + 1] | 883.3 |
| 34.0 | C47H66N12O6S | 926.49 | [M + 1] | 927.3 |
| 35.0 | C49H70N12O7S | 970.52 | [M + 1] | 971.2 |
| 36.0 | C51H74N12O8S | 1014.55 | [M + 1] | 1015.3 |
| 37.0 | C52H66N12O6S | 986.49 | [M + 1] | 987.1 |
| 38.0 | C54H70N12O7S | 1030.52 | [M + 1] | 1031.2 |
| 39.0 | C56H74N12O8S | 1074.55 | [M + 1] | 1075.4 |
| 40.0 | C58H78N12O9S | 1118.57 | [M + 1] | 1119.5 |
| 41.0 | C55H80ClN13O9S | 1133.56 | [M + 1] | 1134.5 |
| 42.0 | C53H76ClN13O8S | 1089.53 | [M + 1] | 1090.5 |
| 43.0 | C49H68ClN13O6S | 1001.48 | [M + 1] | 1002.4 |
| 44.0 | C51H72ClN13O7S | 1045.51 | [M + 1] | 1046.5 |
| 45.0 | C39H51ClN10O5 | 774.37 | [1/2M + 1] | 388.8 |
| 46.0 | C39H52N10O5 | 740.41 | [1/2M + 1] | 371.3 |
| 47.0 | C37H48N10O5 | 712.38 | [M + 1] | 713.4 |
| 48.0 | C38H50N10O5 | 726.40 | [M + 1] | 727.4 |
| 49.0 | C37H47ClN10O5 | 746.34 | [M + 1] | 747.3 |
| 50.0 | C38H49ClN10O5 | 760.36 | [M + 1] | 761.3 |
| 51.0 | C58H76ClN13O8S | 1149.53 | [M + 1] | 1150.5 |
| 52.0 | C56H72ClN13O7S | 1105.51 | [M + 1] | 1106.5 |
| 53.0 | C60H80ClN13O9S | 1193.56 | [M + 1] | 1194.5 |
| 54.0 | C62H84ClN13O10S | 1237.59 | [M + 1] | 1238.5 |
| 55.0 | C37H46N10O5 | 710.37 | [1/2M + 1] | 356.3 |
| 56.0 | C39H50N10O5 | 738.40 | [M + 1] | 739.3 |

### Compound 57.0 4-[3-[4-[2-[[(1S,3S)-3-[[3-chloro-5-[(E)-1-ethylprop-1-enyl]pyrazolo[1,5-a]pyrimidin-7-yl]amino]cyclopentyl]amino]acetyl]piperazin-1-yl]propylamino]-2-(2,6-dioxo-3-piperidyl)isoindoline-1,3-dione

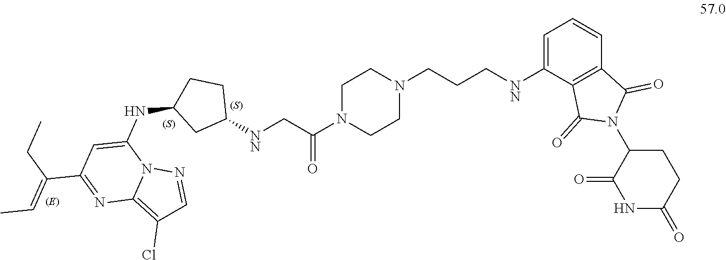

### Step-1. Synthesis of tert-butyl N-[(1S, 3S)-3-[[5-[(E)-1-ethylprop-1-enyl]pyrazolo [1,5-a]pyrimidin-7-yl]amino]cyclopentyl]carbamate (Compound 57.01)

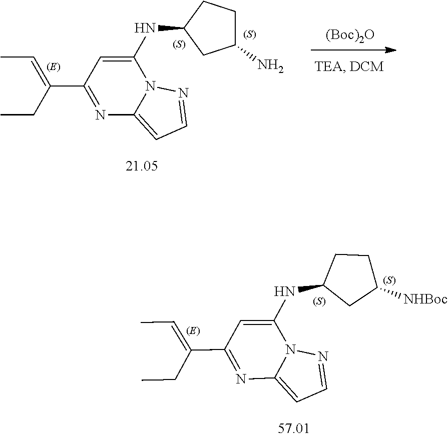

To a solution of (1S,3S)—N3-[5-[(E)-1-ethylprop-1-enyl]pyrazolo[1,5-a]pyrimidin-7-yl]cyclopentane-1, 3-diamine (Compound 21.05) (900 mg, 3.15 mmol) in DCM (10 mL) was added TEA (0.88 mL, 6.3 mmol) and di-tert-butyl dicarbonate (826 mg, 3.8 mmol), the mixture was stirred at 25° C. for 2 h. LCMS showed the starting material (Compound 21.05) was consumed completely and one main peak with desired MS was detected. TLC (petroleum ether:ethyl acetate=2:1, R_{f}=0.56, ultraviolet) indicated the starting material was consumed completely and one new spot formed. The reaction mixture was poured to water (8 mL), the resulting mixture was extracted with dichloromethane (10 mL*3), the combined organic layers were washed with brine (5 mL), dried over anhydrous sodium sulfate and concentrated in vacuum to give a crude product. The crude product was purified by flash column (ISCO 20 g silica, 0-30% ethyl acetate in petroleum ether, gradient over 20 min). Compound tert-butyl N-[(1S, 3S)-3-[[5-[(E)-1-ethylprop-1-enyl]pyrazolo [1,5-a]pyrimidin-7-yl]amino]cyclopentyl]carbamate (Compound 57.01) (1.17 g, 3.035 mmol, 96% yield) was obtained as a pale yellow gum.

ee %=99.32%. LCMS-ESI (pos.) m/z: 386.4 [M⁺H]⁺

¹H NMR (400 MHz, MeOD-d₄) δ=7.98 (d, J=2.3 Hz, 1H), 6.36 (d, J=2.3 Hz, 1H), 6.28 (q, J=7.0 Hz, 1H), 6.17 (s, 1H), 4.58-4.54 (m, 1H), 4.28 (quin, J=6.8 Hz, 1H), 4.15-4.07 (m, 1H), 2.67 (q, J=7.5 Hz, 2H), 2.34 (dtd, J=5.1, 7.7, 12.9 Hz, 1H), 2.19 (dtd, J=5.1, 7.7, 12.8 Hz, 1H), 2.06 (t, J=6.8 Hz, 2H), 1.88 (d, J=7.0 Hz, 3H), 1.80-1.70 (m, 1H), 1.65-1.56 (m, 1H), 1.45 (s, 9H), 1.02 (t, J=7.5 Hz, 3H).

### Step-2. Synthesis of tert-butyl N-[(1S, 3S)-3-[[3-chloro-5-[(E)-1-ethylprop-1-enyl]pyrazolo [1,5-a]pyrimidin-7-yl]amino]cyclopentyl]carbamate (Compound 57.02)

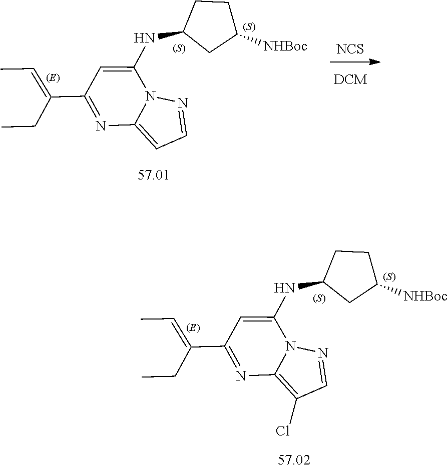

To a solution of tert-butyl N-[(1S,3S)-3-[[5-[(E)-1-ethylprop-1-enyl]pyrazolo[1,5-a]pyrimidin-7-yl]amino]cyclopentyl]carbamate (Compound 57.01) (900 mg, 2.33 mmol) in DCM (45 mL) was slowly added NCS (327 mg, 2.45 mmol) at 0° C. and then the reaction was stirred for 1 h. TLC (petroleum ether:ethyl acetate=3:1, R_{f}=0.5, ultraviolet) indicated trace of the starting material (Compound 57.01) was remaining and some new spots formed. The reaction mixture was concentrated under reduced pressure to give a residue. The residue was purified by flash column (ISCO 20 g silica, 0-20% ethyl acetate in petroleum ether, gradient over 20 min). The compound tert-butyl N-[(1S, 3S)-3-[[3-chloro-5-[(E)-1-ethylprop-1-enyl]pyrazolo [1,5-a]pyrimidin-7-yl]amino]cyclopentyl]carbamate (Compound 57.02) (750 mg, 1.79 mmol, 76% yield) was obtained as pale yellow oil.

LCMS-ESI (pos.) m/z: 420.2 [M⁺H]⁺

¹H NMR (400 MHz, CDCl₃) δ=7.82 (s, 1H), 6.30-6.39 (m, 1H), 6.05 (br d, J=6.75 Hz, 1H), 5.97 (s, 1H), 4.49 (br s, 1H), 4.07-4.14 (m, 2H), 2.62 (q, J=7.50 Hz, 2H), 2.25-2.34 (m, 1H), 2.13-2.24 (m, 1H), 1.99-2.09 (m, 2H), 1.81 (d, J=6.88 Hz, 3H), 1.58-1.71 (m, 1H), 1.45-1.56 (m, 1H), 1.36-1.41 (m, 9H), 0.99 (t, J=7.50 Hz, 3H).

### Step-3. Synthesis of (1S, 3S)—N3-[3-chloro-5-[(E)-1-ethylprop-1-enyl]pyrazolo [1,5-a]pyrimidin-7-yl]cyclopentane-1, 3-diamine (Compound 57.03)

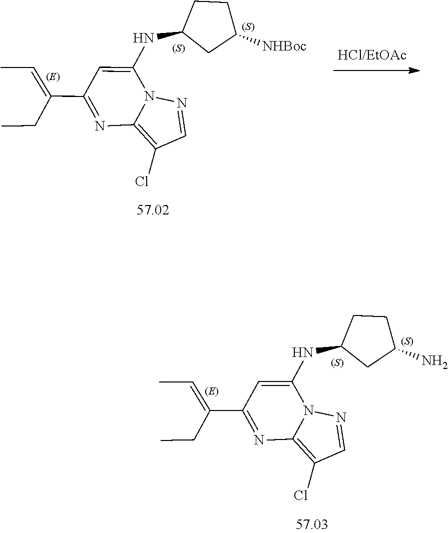

To a solution of tert-butyl N-[(1S,3S)-3-[[3-chloro-5-[(E)-1-ethylprop-1-enyl]pyrazolo[1,5-a]pyrimidin-7-yl]amino]cyclopentyl]carbamate (Compound 57.02)

(750 mg, 1.79 mmol) in HCl/EtOAc (4 M, 3.57 mL, 14.29 mmol) was stirred at 25° C. for 2 h. LCMS showed starting material was consumed completely and main desired MS was detected. The reaction mixture was concentrated under reduced pressure to give a crude product. The crude product (1S,3S)—N3-[3-chloro-5-[(E)-1-ethylprop-1-enyl]pyrazolo[1,5-a]pyrimidin-7-yl]cyclopentane-1,3-diamine (Compound 57.03) (700 mg, 1.96 mmol, crude) was obtained as pale yellow solid, a hydrochloride salt.

LCMS-ESI (pos.) m/z: 320.1 [M⁺H]⁺

### Step-4. Synthesis of tert-butyl 2-[[(1S,3S)-3-[[3-chloro-5-[(E)-1-ethylprop-1-enyl]pyrazolo[1,5-a]pyrimidin-7-yl]amino]cyclopentyl]amino]acetate (Compound 57.04)

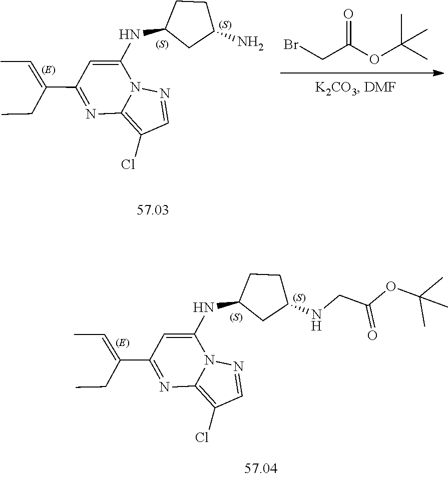

To a solution of (1S,3S)—N3-[3-chloro-5-[(E)-1-ethylprop-1-enyl]pyrazolo[1,5-a]pyrimidin-7-yl]cyclopentane-1,3-diamine; hydrochloride (Compound 57.03) (600 mg, 1.31 mmol) in DMF (6 mL) was added K₂CO₃ (543.8 mg, 3.94 mmol) and tert-butyl 2-bromoacetate (256.2 mg, 1.31 mmol) at 0° C. and then the reaction was stirred at 25° C. for 3 h. LCMS showed trace of starting material was remaining and the desired MS was detected. The mixture was poured into water (10 mL) and extracted with ethyl acetate (3*20 mL). The organic layer was washed with brine (20 mL), dried over anhydrous Na₂SO₄, then filtered and concentrated under reduced pressure. The crude product was purified by flash column (ISCO 20 g silica, 0-40% ethyl acetate in petroleum ether, gradient over 20 min). The compound tert-butyl 2-[[(1S,3S)-3-[[3-chloro-5-[(E)-1-ethylprop-1-enyl]pyrazolo[1,5-a]pyrimidin-7-yl]amino]cyclopentyl]amino]acetate (Compound 57.04) (360 mg, 0.83 mmol, 63% yield) was obtained as yellow oil. LCMS-ESI (pos.) m/z: 434.2 [M⁺H]⁺

### Step-5. Synthesis of 2-[[(1S, 3S)-3-[[3-chloro-5-[(E)-1-ethylprop-1-enyl]pyrazolo[1,5-a]pyrimidin-7-yl]amino]cyclopentyl]amino]acetic acid (Compound 57.05)

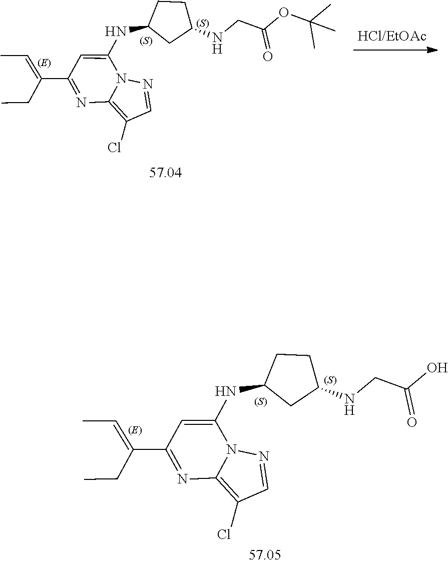

A solution of tert-butyl 2-[[(1S,3S)-3-[[3-chloro-5-[(E)-1-ethylprop-1-enyl]pyrazolo[1,5-a]pyrimidin-7-yl]amino]cyclopentyl]amino]acetate (Compound 57.04) (200 mg, 0.46 mmol) in 4 M HCl in EtOAc (3 mL, 12 mmol) was stirred for 2 h at 20° C. LC-MS showed the starting material (Compound 57.04) was consumed completely and the desired mass was detected. The mixture was concentrated in vacuum. The residue was purified by prep-HPLC (Phenomenex luna C18 80*40 mm*3 um column; 22-42% acetonitrile in a 0.04% HCl solution, 7 min gradient). 2-[[(1S,3S)-3-[[3-chloro-5-[(E)-1-ethylprop-1-enyl]pyrazolo[1,5-a]pyrimidin-7-yl]amino]cyclopentyl]amino]acetic acid (Compound 57.05) (100 mg, 0.2646 mmol, 57% yield) was obtained as a yellow solid.

ee %=96.86%. LCMS-ESI (pos.) m/z: 378.1 [M⁺H]⁺

¹H NMR (400 MHz, MeOD-d₄) δ=8.24-8.29 (m, 1H), 6.63-6.68 (m, 1H), 6.33-6.42 (m, 1H), 4.80-4.87 (m, 1H), 3.99 (s, 2H), 3.88-3.97 (m, 1H), 2.70-2.80 (m, 2H), 2.38-2.49 (m, 3H), 2.28-2.38 (m, 1H), 1.87-2.00 (m, 5H), 1.02-1.12 (m, 3H).

### Step-6. Synthesis of 4-[3-[4-[2-[[(1S,3S)-3-[[3-chloro-5-[(E)-1-ethylprop-1-enyl]pyrazolo[1,5-a]pyrimidin-7-yl]amino]cyclopentyl]amino]acetyl]piperazin-1-yl]propylamino]-2-(2,6-dioxo-3-piperidyl)isoindoline-1,3-dione (Compound 57.0)

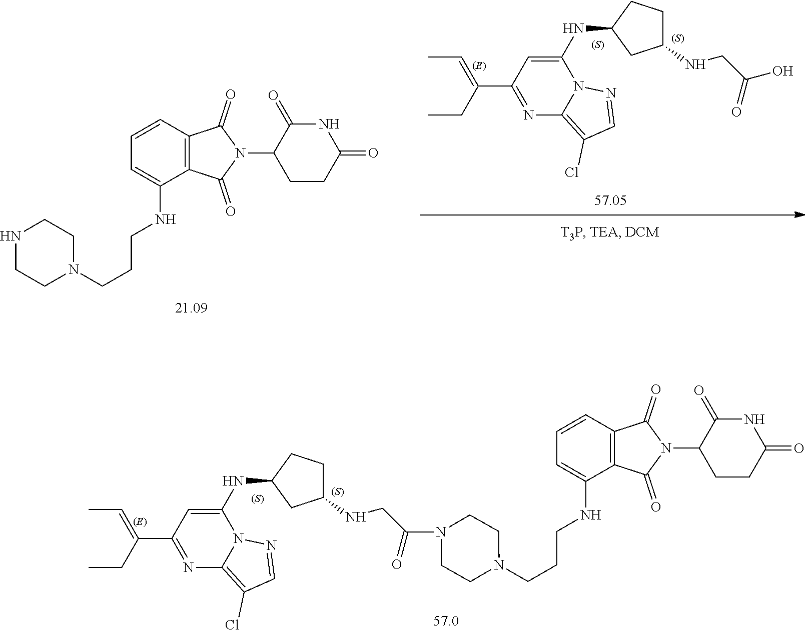

To a solution of 2-(2,6-dioxo-3-piperidyl)-4-(3-piperazin-1-ylpropylamino)isoindoline-1,3-dione; hydrochloride (Compound 21.09) (27.69 mg, 0.06 mmol) in DCM (0.5 mL) was added 2-[[(1S,3S)-3-[[3-chloro-5-[(E)-1-ethylprop-1-enyl]pyrazolo[1,5-a]pyrimidin-7-yl]amino]cyclopentyl]amino]acetic acid (Compound 57.05) (20 mg, 0.05 mmol), T3P (101.04 mg, 0.16 mmol) and TEA (0.52 mL, 3.7 mmol) and then the reaction was stirred at 25° C. for 1 h. LCMS showed starting material was consumed completely and main desired MS was detected. The reaction was concentrated under reduced pressure to give a residue. The residue was purified by prep-HPLC (Phenomenex luna C18 80*30 mm*3 um column; 15-40% acetonitrile in a 0.1% TFA solution, 8 min gradient). The compound 4-[3-[4-[2-[[(1S,3S)-3-[[3-chloro-5-[(E)-1-ethylprop-1-enyl]pyrazolo[1,5-a]pyrimidin-7-yl]amino]cyclopentyl]amino]acetyl]piperazin-1-yl]propylamino]-2-(2,6-dioxo-3-piperidyl)isoindoline-1,3-dione (Compound 57.0) (7 mg, 0.009 mmol, 17% yield) was obtained as yellow solid. LCMS-ESI (pos.) m/z: 380.2 [M+2H]²+

¹H NMR (400 MHz, METHANOL-d₄) δ=8.01-8.08 (m, 1H), 7.54-7.63 (m, 1H), 7.06-7.16 (m, 2H), 6.27-6.41 (m, 2H), 5.05 (br dd, J=12.34, 5.42 Hz, 1H), 4.41-4.56 (m, 1H), 4.12-4.27 (m, 2H), 3.61-3.95 (m, 4H), 3.49 (br t, J=6.20 Hz, 3H), 3.31-3.44 (m, 6H), 2.64-2.92 (m, 5H), 2.39-2.49 (m, 2H), 2.29-2.37 (m, 2H), 2.06-2.15 (m, 3H), 1.80-1.99 (m, 5H), 1.04 (t, J=7.45 Hz, 3H).

### Compound 58.0 4-[2-[4-[2-[[(1S,3S)-3-[[3-chloro-5-[(E)-1-ethylprop-1-enyl]pyrazolo[1,5-a]pyrimidin-7-yl]amino]cyclopentyl]amino]acetyl]piperazin-1-yl]ethylamino]-2-(2,6-dioxo-3-piperidyl)isoindoline-1,3-dione

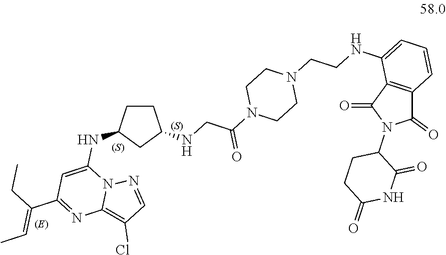

The Compound 58.0 was prepared using the synthetic procedures as described above in Compound 57.0 and corresponding reagents.

LCMS-ESI (pos.) m/z: 373.2 [M+2H]²⁺

¹H NMR (400 MHz, MeOD-d₄) δ=8.06 (s, 1H), 7.60-7.67 (m, 1H), 7.11-7.20 (m, 2H), 6.21-6.41 (m, 2H), 5.08 (br dd, J=12.58, 5.42 Hz, 1H), 4.49 (br d, J=3.93 Hz, 1H), 4.15-4.24 (m, 2H), 3.70-3.95 (m, 7H), 3.44 (br d, J=5.01 Hz, 6H), 2.64-2.93 (m, 5H), 2.40-2.51 (m, 2H), 2.29-2.38 (m, 2H), 2.07-2.16 (m, 1H), 1.79-2.03 (m, 5H), 1.01-1.10 (m, 3H).

### Compound 59.0 4-[4-[4-[2-[[(1S,3S)-3-[[3-chloro-5-[(E)-1-ethylprop-1-enyl]pyrazolo[1,5-a]pyrimidin-7-yl]amino]cyclopentyl]amino]acetyl]piperazin-1-yl]butylamino]-2-(2,6-dioxo-3-piperidyl)isoindoline-1,3-dione

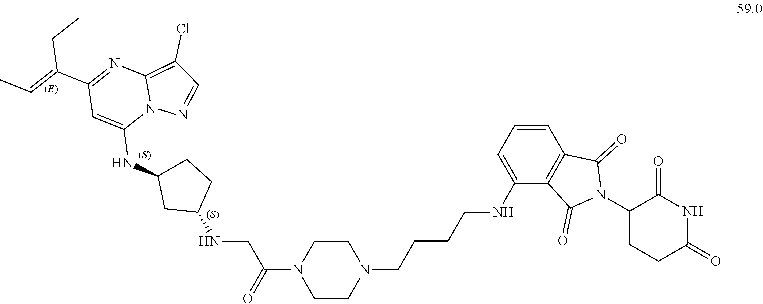

The Compound 59.0 was prepared using the synthetic procedures as described above in Compound 57.0 and corresponding reagents.

LCMS-ESI (pos.) m/z: 387.3 [M+2H]²⁺

¹H NMR (400 MHz, MeOD-d₄) δ=8.03 (d, J=5.84 Hz, 1H), 7.53-7.62 (m, 1H), 7.06-7.11 (m, 2H), 6.33-6.42 (m, 1H), 6.30 (br d, J=7.99 Hz, 1H), 5.06 (dd, J=12.34, 5.42 Hz, 1H), 4.41-4.54 (m, 1H), 4.20 (br s, 2H), 3.60-3.94 (m, 4H), 3.34-3.50 (m, 5H), 3.16-3.29 (m, 4H), 2.65-2.92 (m, 5H), 2.41-2.49 (m, 2H), 2.30-2.37 (m, 2H), 2.06-2.16 (m, 1H), 1.91 (br d, J=7.03 Hz, 7H), 1.73-1.80 (m, 2H), 1.05 (t, J=7.45 Hz, 3H).

### Experimental Methods: CDK9 Degradation by HiBit Assay

Exemplary compounds of the invention were tested for degradation of CDK9. The compounds were tested in 10-dose DC50 duplicate mode with a 3-fold serial dilution starting at 10 μM by HiBit assay (Promega, Inc) as a direct measurement of CDK9 presence, with incubation time of 24 hours using either NCIH211 cells with endogenously tagged N-terminal CDK9-HiBit or NCIH1048 cells with endogenously tagged N-terminal CDK9-HiBit. A control compound (THAL-SNS-032, CAS 2139287-33-3) was tested in 10-dose DC50 mode with 3-fold serial dilution starting at 10 μM. Results for the tested compounds are shown in the Table 2. Data was normalized to positive (medium only) and negative (cells with no treatment) controls and curve fits were analyzed with CDK9 as % response vs. log [compound concentration] using a 4-parameter logistic model by GraphPad software. For Table 2: A is <100, B is <1000 C is <10,000, D is >10,000 and ND is Not Determined.

### HiBit DC₅₀ Data for Representative Compounds of the Present Invention

TABLE 2

| | HiBit-NCIH211.2 | HiBit-NCIH1048.3 |
|---|---|---|
| Compound | DC₅₀ (nM) | DC₅₀ (nM) |
| THAL-SNS-032 | 53 | 11 |
| 1.0 | D | ND |
| 2.0 | D | ND |
| 3.0 | D | ND |
| 4.0 | D | ND |
| 5.0 | C | B |
| 6.0 | B | A |
| 7.0 | D | ND |
| 8.0 | D | ND |
| 9.0 | C | ND |
| 10.0 | C | ND |
| 11.0 | C | ND |
| 12.0 | D | ND |
| 13.0 | C | ND |
| 14.0 | D | ND |
| 15.0 | C | ND |
| 16.0 | D | ND |
| 17.0 | C | ND |
| 18.0 | D | ND |
| 19.0 | C | ND |
| 20.0 | D | ND |
| 21.0 | A | A |
| 22.0 | D | ND |
| 23.0 | C | ND |
| 24.0 | B | ND |
| 25.0 | D | ND |
| 26.0 | D | ND |
| 27.0 | D | ND |
| 28.0 | D | ND |
| 29.0 | D | ND |
| 30.0 | D | ND |
| 31.0 | D | ND |
| 32.0 | D | ND |
| 33.0 | D | ND |
| 34.0 | D | ND |
| 35.0 | D | ND |
| 36.0 | D | ND |
| 37.0 | D | ND |
| 38.0 | D | ND |
| 39.0 | D | ND |
| 40.0 | D | ND |
| 41.0 | D | ND |
| 42.0 | D | ND |
| 43.0 | D | ND |
| 44.0 | D | ND |
| 45.0 | A | A |
| 46.0 | B | A |
| 47.0 | B | A |
| 48.0 | B | A |
| 49.0 | A | ND |
| 50.0 | A | A |
| 51.0 | C | C |
| 52.0 | D | C |
| 53.0 | C | C |
| 54.0 | C | C |
| 55.0 | A | A |
| 56.0 | A | A |
| 57.0 | A | A |
| 58.0 | A | A |
| 59.0 | A | A |

In the above table A is <100, B is <1000, C is <10,000, D is >10,000, and ND is Not Determined.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

1. A compound of formula (1):
X-L-Y (I)
wherein:
X is an E3 ubiquitin ligase binding ligand; and
L is a linking group covalently bonded to X and Y;
Y is a cyclin dependent kinase 9 (CDK9) binding ligand of formula (II): 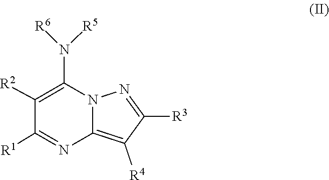
or a pharmaceutically acceptable salt thereof;
R¹ is a C1-C6 alkyl, C3-C6 cycloalkyl, C3-C6 cycloalkenyl, alkenyl, alkynyl, aryl, heteroaryl, or heterocyclyl, optionally substituted at any position with one or more of D, halo, R⁷CO₂R⁸, CO₂R⁸, CO₂H, R⁷CO₂H, NH₂, NHR⁸, OH, OR⁸, SH, SR⁸, NHCOR⁸, NHSO₂R⁸, SO₂NH₂, SO₂NHR⁸,
or R¹ and R² together form a fused C5-C6 cycloaryl, optionally substituted at any position with one or more of D, halo, NH₂, NHR⁸, NR⁷R⁸, OH, OR⁸, SH, SR⁸, NHCOR⁸, NHSO₂R⁸, SO₂NH₂, or SO₂NHR⁸;
R², R³, and R⁵ are independently H, D, halo, or C1-C5 alkyl or C3-C6 cycloalkyl optionally substituted at any position with one more of D, halo, R⁷CO₂R⁸, CO₂R⁸, C02H, R⁷CO₂H, NH₂, NHR⁸, OH, OR⁸, SH, SR⁸, NHCOR⁸, NHSO₂R⁸, SO₂NH₂, SO₂NHR⁸,
or R² and R⁵ together form a fused C5-C6 cycloaryl, optionally substituted at any position with one or more of D, halo, R⁷CO₂R⁸, CO₂R⁸, CO₂H, R⁷CO₂H, NH₂, NHR⁸, OH, OR⁸, SH, SR⁸, NHCOR⁸, NHSO₂R⁸, SO₂NH₂, SO₂NHR⁸;
R⁴ is H, D, halo, C1-C5 alkyl, C3-C6 cycloalkyl, cyano, hydroxyl, or —O—(C1-C5 alkyl), optionally substituted at any position with one more of D, halo, R⁷CO₂R⁸, CO₂R⁸, CO₂H, R⁷CO₂H, NH₂, NHR⁸, OH, OR⁸, SH, SR⁸, NHCOR⁸, NHSO₂R⁸, SO₂NH₂, SO₂NHR⁸;
R⁶ is H or D;
R⁷ is (CH₂)ₙ and n is an integer from 1 to 6; and
R⁸ is C1-C6 alkyl or C3-C6 cycloalkyl, optionally substituted at any position with one or more of D, halo, OH, SH, or NH₂.

2. The compound of claim 1, wherein Y is covalently attached to L through R⁵.

3. The compound of claim 1, wherein R⁵ is an optionally substituted C3-C6 cycloalkyl group.

4. The compound of claim 3, wherein R⁵ is an optionally substituted C5 cycloalkyl group.

5. The compound of claim 4, wherein R⁵ has a structure selected from the group consisting of:

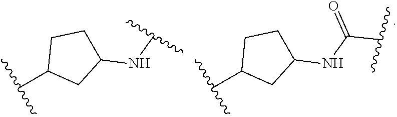 [SMILES: *NC1CCC(*)C1 ; *C(=O)NC1CCC(*)C1]

6. The compound of claim 1, wherein R¹ is a C1-C6 alkyl group.

7. The compound of claim 6, wherein R¹ has a structure selected from the group consisting of:

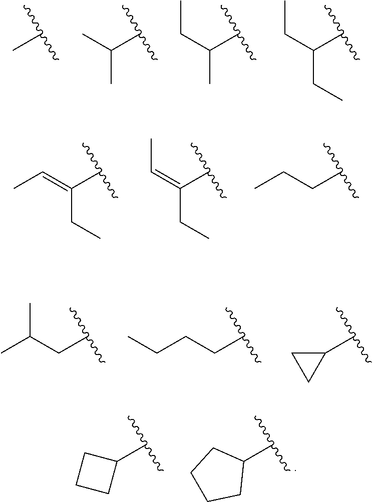

8. The compound of claim 1, wherein R², R³, and R⁶ are each independently H or D.

9. The compound of claim 1, wherein R⁴ is halo, cyano, or methyl.

10. The compound of claim 1, wherein R⁴ is H or D.

11. The compound of claim 1, wherein Y has a structure selected from the group consisting of:

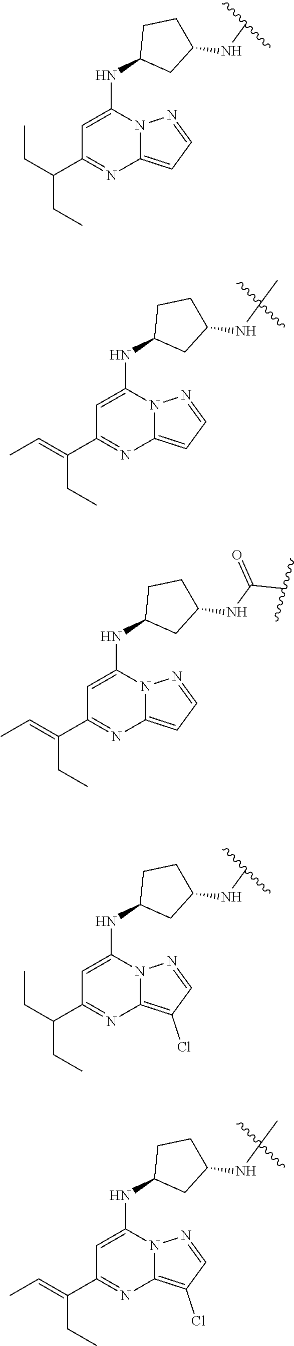 [SMILES: *N[C@H]1CC[C@H](Nc2cc(C(CC)CC)nc3ccnn23)C1 ; *N[C@H]1CC[C@H](Nc2cc(/C(=C/C)CC)nc3ccnn23)C1 ; *C(=O)N[C@H]1CC[C@H](Nc2cc(/C(=C/C)CC)nc3ccnn23)C1 ; *N[C@H]1CC[C@H](Nc2cc(C(CC)CC)nc3c(Cl)cnn23)C1 ; *N[C@H]1CC[C@H](Nc2cc(/C(=C/C)CC)nc3c(Cl)cnn23)C1]

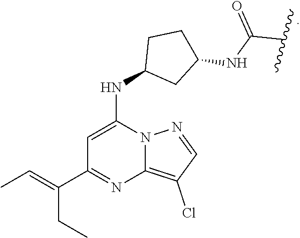 [SMILES: *C(=O)N[C@H]1CC[C@H](Nc2cc(/C(=C/C)CC)nc3c(Cl)cnn23)C1]

12. The compound of claim 1, wherein X is a cereblon (CRBN) binding ligand.

13. (canceled)

14. The compound of claim 12, wherein X has a structure derived by removal of a hydrogen atom from a structure selected from the group consisting of:

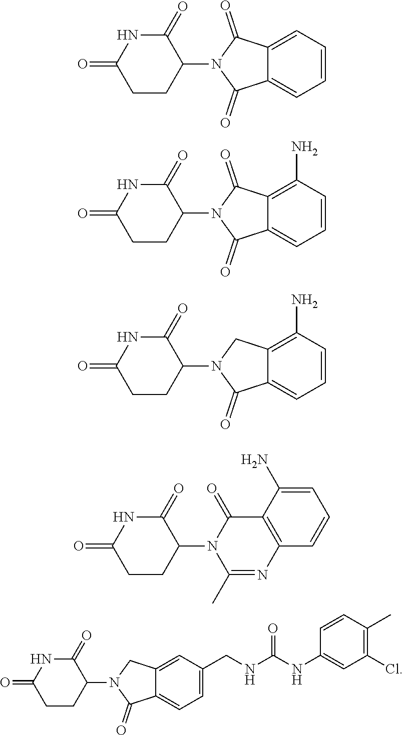

15. The compound of claim 14, wherein X has the structure

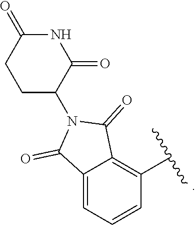 [SMILES: *c1cccc2c1C(=O)N(C1CCC(=O)NC1=O)C2=O]

16. The compound of claim 1, wherein X is a Von Hippel-Lindau (VHL) binding ligand.

17. (canceled)

18. The compound of claim 16, wherein X has a structure derived by removal of a hydrogen atom from a structure selected from the group consisting of:

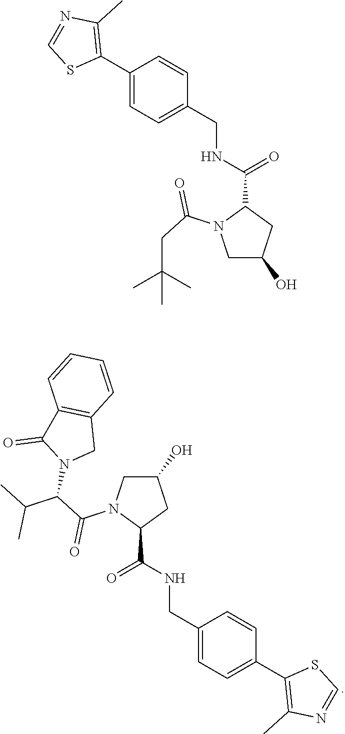

19. The compound of claim 1, wherein L has a structure of formula (III) 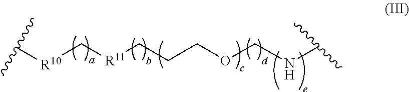
wherein:
a, b, c, and d are each independently an integer from 0 to 6,
e is 0 or 1,
R¹⁰ is absent or has a structure selected from the group consisting of: 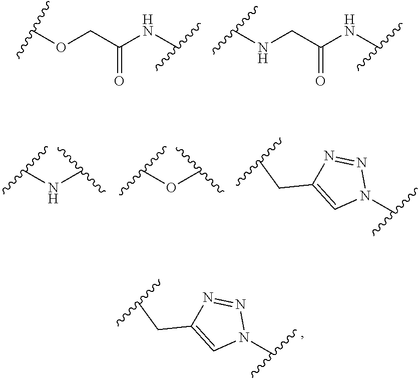
and
R¹¹ is absent or has a structure selected from the group consisting of:

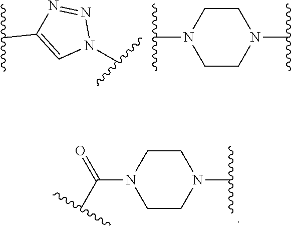

20. The compound of claim 1, wherein the compound has a structure selected from the group consisting of Compounds X.0, wherein X is an integer ranging from 1 to 59.

21. A method of treating a subject for a CDK9-mediated disease, comprising:
administering a therapeutically effective dose of a pharmaceutical composition comprising a compound of claim 1.

22. The method of claim 21, wherein the CDK9-mediated disease is cancer.

23. (canceled)

24. (canceled)

25. (canceled)
